(19) United States
(12) Patent Application Publication
(10) Pub. No.: US 2024/0192224 A1
(43) Pub. Date: Jun. 13, 2024

(54) NEUTRALIZING ANTIBODY ASSAYS AND COMPOSITIONS

(71) Applicant: ADITXT, INC., Mountain View, CA (US)

(72) Inventors: Ge CHEN, Mountain View, CA (US); Hong LIU, Mountain View, CA (US); Shahrokh SHABAHANG, Mountain View, CA (US)

(21) Appl. No.: 18/553,490

(22) PCT Filed: Apr. 1, 2022
(86) PCT No.: PCT/US2022/023157

§ 371 Date: Sep. 29, 2023

Related U.S. Application Data
(60) Provisional application No. 63/283,161, filed on Nov. 24, 2021.
(60) Provisional application No. 63/170,130, filed on Apr. 2, 2021.

(51) Int. Cl. G01N 33/68; G01N 33/543; G01N 33/569; G01N 33/58

(52) U.S. Cl. CPC G01N 33/6854; G01N 33/54313; G01N 33/56983; G01N 33/582; G01N 2469/20; G01N 2470/10

(57) ABSTRACT

The present disclosure relates generally to compositions and methods for assaying antibodies. More specifically, the disclosure relates to assays that detect antibodies that have neutralizing activity against SARS-CoV-2.

### TECHNICAL FIELD

The present disclosure relates generally to compositions and methods for assaying antibodies. More specifically, the disclosure relates to assays that detect antibodies that have neutralizing activity against SARS-CoV-2.

### BACKGROUND

Infection with a virus, such as SARS-CoV-2, initiates a virus-specific immunity that includes a T cell response and the production of antibodies to eliminate the virus from the body of the infected organism. Cytotoxic T cells can kill virus-infected cells, and virus-specific antibodies can bind to the virus and prevent it from spreading between cells. Not all virus-specific antibodies, however, prevent the virus particle to which they are bound from infecting a cell. Neutralizing antibodies are a subset of virus-specific antibodies that block infection by interfering with the binding and/or cell entry of virus particles. This ability to block infection is referred to as “neutralizing activity”. While there are assays available for detecting the presence of antibodies to virus particles, it is more difficult and labor-intensive to determine whether the antibodies identified have neutralizing properties.

There are two types of assays currently available for detecting the presence of neutralizing antibodies. The first type of assay is an ELISA-based direct binding assay, such as the cPass™ assay by Genscript. This type of assay is based on the competition for binding to viral proteins between the viral receptor and neutralizing antibodies. For example, to detect SARS-CoV-2 neutralizing antibodies, an ELISA plate is coated with angiotensin-converting enzyme 2 (ACE-2), a SARS-CoV-2 receptor. HRP-conjugated SARS-CoV-2 S protein receptor binding domain (RBD) and sample containing antibodies are added to the plate. The degree to which the sample antibodies inhibit the interaction between the ACE-2 and the RBD can be measured by the colorimetric signal generated by the binding of HRP-RBD to the ACE-2-coated plate. This type of assay is relatively simple, but it takes about one and a half days to complete and detects only neutralizing antibodies that bind to the S protein RBD. Neutralizing antibodies that bind to other regions of the SARS-CoV-2 S protein are not detected by this assay.

The second type of assay is a cell-based assay, which is the gold standard for detection of neutralizing antibodies. Some cell-based assays use a SARS-CoV-2 virus clinical isolate or a recombinant SARS-CoV-2 that encodes a reporter protein. When introduced to a target cell population in the presence of a sample containing antibodies, the degree of inhibition of infection can be directly measured. Cell-based assays that use live SARS-CoV-2 virus require extensive safety precautions and may take up to five days to complete. Another type of cell-based assay uses a less hazardous live virus, such as vesicular stomatitis virus (VSV), which has been pseudotyped to express SARS-CoV-2 S protein on its surface. The degree to which infection of target cells by the pseudotyped virus is inhibited by the antibodies present in a test sample is then measured. While using pseudotyped virus provides some safety benefits, these assays still require significant safety precautions, and they typically take two to three days to complete. Because cell-based assays use live cells as the infectious target, these assays produce results that are highly biologically relevant. However, cell-based assays are complicated, require significant expertise to carry out, and have a lengthier turn-around time than is desirable for many applications.

A simple, fast, and reliable method for detecting antibodies with neutralizing activity would be useful for many purposes, including providing a better understanding of a patient's immune status and identifying antibodies that could be effectively used in anti-viral therapies.

### BRIEF SUMMARY

The present disclosure provides a method of detecting SARS-CoV-2 neutralizing antibodies, the method comprising: a) combining at least two types of identifiably labelled microparticles conjugated to at least two different SARS-CoV-2 proteins or a fragment thereof, at least one of which comprises a SARS-CoV-2 S protein or fragment thereof, with a detectably labelled SARS-CoV-2 S protein receptor or a fragment thereof, and a test sample; b) detecting identifiable labels and the detectable label both associated with microparticles to generate detection data; and c) combining or measuring the detection data to generate a test sample property relating to the presence or absence of or amount of neutralizing antibodies in the test sample.

The disclosure further provides a method of detecting SARS-CoV-2 neutralizing antibodies, the method comprising: a) combining at least one identifiably labelled microparticle conjugated to a SARS-CoV-2 S protein or a fragment thereof and, optionally, a second identifiably labelled microparticle conjugated to another SARS-CoV-2 S protein or a fragment thereof or SARS-CoV-2 nucleoprotein (NP) or a fragment thereof, with a detectably labelled SARS-CoV-2 S protein receptor or a fragment thereof, and a test sample; b) detecting identifiable label and the detectable label both associated with microparticles to generate detection data; and c) combining or measuring the detection data to generate a test sample property relating to the presence or absence of or amount of neutralizing antibodies in the test sample.

The disclosure further provides a method of detecting SARS-CoV-2 neutralizing antibodies, the method comprising: a) combining identifiably labelled microparticles conjugated to a SARS-CoV-2 S protein receptor or a fragment thereof with a detectably labelled SARS-CoV-2 S protein or a fragment thereof, and a test sample; b) detecting the identifiable label and the detectable label both associated with microparticles to generate detection data; and c) combining or measuring the detection data to generate a test sample property relating to the presence or absence of or amount of neutralizing antibodies in the test sample.

The disclosure further provides a method of detecting SARS-CoV-2 neutralizing antibodies for at least two SARS-CoV-2 variants, the method comprising: a) combining at least two types of identifiably labelled microparticles conjugated to at least two different SARS-CoV-2 S proteins, RBDs or fragment thereof from at least two different SARS-CoV-2 variants with a detectably labelled SARS-CoV-2 S protein receptor or a fragment thereof, and a test sample; b) detecting identifiable labels and the detectable label both associated with microparticles to generate detection data; and c) combining or measuring the detection data to generate a test sample property relating to the presence or absence of or amount of neutralizing antibodies for both variants in the test sample.

The disclosure additionally provides a method of detecting SARS-CoV-2 neutralizing antibodies for at least two SARS-CoV-2 variants, the method comprising: a) combining identifiably labelled microparticles conjugated to a SARS-CoV-2 S protein receptor or a fragment thereof with at least two different detectably labelled SARS-CoV-2 S proteins, RBDs or fragment thereof from at least two different SARS-CoV-2 variants, and a test sample; b) detecting the identifiable label and the detectable labels both associated with microparticles to generate detection data; and c) combining or measuring the detection data to generate a test sample property relating to the presence or absence of or amount of neutralizing antibodies in the test sample.

The disclosure additionally provides a kit for detecting SARS-CoV-2 antibodies, the kit comprising: a first type of identifiably labelled microparticle conjugated to a SARS-CoV-2 S protein or a fragment thereof; a detectably labelled SARS-CoV-2 S protein receptor or a fragment thereof; and instructions for use.

The disclosure further provides a kit for detecting SARS-CoV-2 antibodies, the kit comprising: an identifiably labelled microparticle conjugated to a SARS-CoV-2 S protein receptor or a fragment thereof; a detectably labelled SARS-CoV-2 S protein or a fragment thereof; and instructions for use.

The disclosure additionally provides a composition comprising a mixture of at least two types of identifiable microparticles, a first type conjugated to a first SARS-CoV-2 S protein or fragment thereof, and a second type conjugated to a second fragment of SARS-CoV-2 S protein, which is different from the first fragment and, optionally, a third type of identifiable microparticle conjugated to a third SARS-CoV-2 nucleoprotein (NP) or a fragment thereof, and, further optionally, a fourth type of identifiable microparticle conjugated to a full-length SARS-CoV-2 S protein.

The disclosure further provides a composition comprising a mixture of at least one identifiable microparticle conjugated to a SARS-CoV-2 S protein or fragment thereof, and optionally, an additional type of identifiable microparticle conjugated to a third SARS-CoV-2 nucleoprotein (NP) or a fragment thereof, and further optionally, an additional type of identifiable microparticle conjugated to a full-length SARS-CoV-2 S protein.

The disclosure further provides a composition comprising a mixture of at least two types of identifiable microparticles, a first type conjugated to a first SARS-CoV-2 S protein or fragment thereof, and a second type conjugated to a second fragment of SARS-CoV-2 S protein, which is different from the first fragment or to a second SARS-CoV-2 S protein from a different variant of SARS-CoV-2 than the first SARS-CoV-2 S protein.

The disclosure further provides a composition comprising a mixture of at least one first type of identifiable microparticle conjugated to a SARS-CoV-2 S protein receptor or fragment thereof, optionally human angiotensin-converting enzyme 2 (ACE-2) or a fragment thereof.

### BRIEF DESCRIPTION OF THE DRAWINGS

FIG. 1 is a schematic representation of the wild-type full-length SARS-CoV-2 S protein, showing the various domains thereof. SS indicates signal sequence. NTD indicates N-terminal domain. RBD indicates receptor binding domain. FP indicates fusion peptide. HR1 indicates heptad repeat 1. CH indicates central helix. CD indicates connector domain. HR2 indicates heptad repeat 2. TM indicates transmembrane domain. CT indicates cytoplasmic tail. S1 and S2 indicate subdomains 1 and 2, respectively, while S1/S2 indicates the protease cleavage site that separates the two subdomains. The extracellular domain is also indicated.

FIG. 2 is a flow chart of one exemplary assay according to the present disclosure, referred to as Platform 1.

FIG. 3 is a schematic diagram of materials usable in the assay method of FIG. 2.

FIG. 4 is a flow chart of another exemplary assay according to the present disclosure, referred to as Platform 2.

FIG. 5 is a schematic diagram of materials usable in the assay of FIG. 4.

FIG. 6 is a flow chart of another exemplary assay according to the present disclosure, referred to as Platform 3.

FIG. 7 is a schematic diagram of materials usable in the assay of FIG. 6.

FIG. 8 is a flow chart of another exemplary assay according to the present disclosure, referred to as Platform 4.

FIG. 9 is a schematic diagram of materials usable in the assay of FIG. 8.

FIG. 10A and FIG. 10B show the results of neutralizing antibody assays on human plasma samples using Platform 1 (RBD-conjugated microparticles and labelled ACE-2) and Platform 2 (ACE-2-conjugated microparticles and labelled RBD). FIG. 10A shows results using 0.5 μl plasma samples. FIG. 10B shows results using 1.0 μl plasma samples. MFI indicates median fluorescence intensity.

FIG. 11A and FIG. 11B show the results of neutralizing antibody assays on human plasma samples using Platform 1 (RBD-conjugated microparticles and labelled ACE-2). FIG. 11A shows results using samples from patients never exposed to SARS-CoV-2. FIG. 11B shows results using samples from patients who tested positive for SARS-CoV-2 infection by RT-PCR.

FIG. 12 shows the results of neutralizing antibody assays carried out with serial dilutions of plasma samples using Platform 1 (RBD-conjugated microparticles and labelled ACE-2).

FIG. 13 shows the results of ACE-2/RBD binding competition assays.

FIG. 14 shows a comparison between the Platform 1 neutralizing antibody assay and an ELISA-based neutralizing antibody assay.

FIG. 15 shows the results of neutralizing antibody assays on plasma samples using the three-microparticle Platform 3 (multiplex) assay.

FIG. 16 shows the results of neutralizing antibody assays on serum samples using the three-microparticle Platform 3 (multiplex) assay. Neutralization as measured using RBD-coated microparticles is shown in the left panel. Neutralization as measured using S1-coated microparticles is shown in the right panel. SARS-CoV-2 antibody negative samples are indicated with (−) and SARS-CoV-2 antibody positive samples are indicated with (+).

FIG. 17A and FIG. 17B show the results of neutralizing antibody assays on plasma samples collected by finger-stick using the three-microparticle Platform 3 (multiplex) assay. Neutralization as measured using RBD-coated microparticles is shown in FIG. 17A. Neutralization as measured using S1-coated microparticles is shown in FIG. 17B.

FIG. 18A and FIG. 18B show a comparison between a two-step process and a one-step process for the three-microparticle Platform 3 (multiplex) neutralizing antibody assay. Comparison of the neutralization assay results for the two-step and one-step processes using S1-coated microparticles is shown in FIG. 18A. Comparison of the neutralization assay results for the two-step and one-step processes using RBD-coated microparticles is shown in FIG. 18B.

FIG. 19 shows the results of neutralizing antibody assays on plasma samples using microspheres conjugated to full-length SARS-CoV-2 S protein.

FIG. 20A and FIG. 20B show a comparison of the three-microparticle and four-microparticle versions of the Platform 3 (multiplex) neutralizing antibody assay. The correlation between the results for the RBD-conjugated microspheres using the three-microparticle assay and the four-microparticle assay is shown in FIG. 20A. The correlation between the results for the S1-conjugated microspheres using the three-microparticle assay and the four-microparticle assay is shown in FIG. 20B.

FIG. 21 shows a comparison of the results from two different types of microparticles used in the four-microparticle Platform 3 (multiplex) assay. The four-microparticle assay was carried out as described in Example 12. Comparison of the results from S1-conjugated microspheres and full-length S protein-conjugated microspheres is shown.

FIG. 22A shows results for detection of anti-SARS-CoV-2 variant Abs in samples from non-exposed subjects. FIG. 22B shows results for detection of anti-SARS-CoV-2 variant Abs in samples from SARS-CoV-2 convalescent subjects.

FIG. 22C shows results for detection of anti-SARS-CoV-2 variant Abs in samples from vaccinated subjects. FIG. 22D shows results for detection of anti-SARS-CoV-2 variant Abs in samples from SARS-CoV-2 booster subjects.

FIG. 23A shows results for detection of anti-SARS-CoV-2 variant NAbs in samples from non-exposed subjects. FIG. 23B shows results for detection of anti-SARS-CoV-2 variant NAbs in samples from SARS-CoV-2 convalescent subjects. FIG. 23C shows results for detection of anti-SARS-CoV-2 variant NAbs in samples from vaccinated subjects. FIG. 23D shows results for detection of anti-SARS-CoV-2 variant NAbs in samples from SARS-CoV-2 booster subjects.

FIG. 24A shows anti-SARS-CoV-2 Abs detected in positive and negative samples in a Platform 1 type assay. FIG. 24B shows anti-SARS-CoV-2 NAbs detected in the same samples in a Platform 1 type assay.

FIG. 25A shows anti-SARS-CoV-2 Abs detected in the positive and negative samples used in FIG. 24A in a Platform 2 type assay. FIG. 25B shows anti-SARS-CoV-2 NAbs detected in the same samples in a Platform 2 type assay.

### DETAILED DESCRIPTION

The present disclosure provides methods for assaying antibodies and related compositions, systems, and kits. More specifically, the disclosure provides assays to detect antibodies that have neutralizing activity against SARS-CoV-2. Such assay may also be referred to as neutralizing antibody assays. The assay may be conducted using a test sample from a subject.

In the process of viral infection, SARS-CoV-2 spike (S) protein mediates the binding of the virus to angiotensin-converting enzyme 2 (ACE-2) on the cell surface. See Shang et al., Cell entry mechanisms of SARS-CoV-2, 117 PNAS 11727 (2020) and Huang, et al., Structural and functional properties of SARS-CoV-2 spike protein, 41 Acta Pharm. Sinica 1141 (2020). The receptor binding domain (RBD) of S protein interacts with ACE-2 to promote the fusion of viral and host cell membranes and, thereby, virus entry into the host cell. SARS-CoV-2 neutralizing antibodies bind to a SARS-CoV-2 protein in a manner that blocks the interaction between ACE-2 and the RBD, thereby preventing host cells from being infected with the SARS-CoV-2 virus. SARS-CoV-2 neutralizing antibodies that bind RBD appear to be the most effective at blocking the interaction between the RBD and ACE-2, however some antibodies that recognize epitopes in the S protein outside the RBD have also been shown to have neutralizing activity. See, e.g., Chi et al., A neutralizing human antibody binds to the N-terminal domain of the Spike protein of SARS-CoV-2, 369 Science 650 (2020).

In some embodiments, a neutralizing SARS-CoV-2 antibody may block the interaction between ACE-2 and the RBD by blocking the binding of RBD to ACE-2.

Antibodies to SARS-CoV-2 are detectable in blood samples approximately one week after initial infection, although neither the length of time required for production of neutralizing antibodies or the duration of time during which neutralizing antibodies are present post-infection is well characterized. Studies have shown that the level of total anti-SARS-CoV-2 antibodies in a patient is not always proportional to the degree of virus neutralization conferred because not all binding antibodies are neutralizing, and the proportion of total antibodies that are neutralizing antibodies is highly variable.

The present disclosure provides assays for specific detection of SARS-CoV-2 neutralizing antibodies and related compositions and kids. More specifically, the disclosure relates to assays that detect neutralizing antibodies using a microparticle platform.

In the present description, any concentration range, percentage range, ratio range, or integer range is to be understood to include the endpoints of the recited range and the value of any integer within the recited range and, when appropriate, fractions thereof (such as one tenth and one hundredth of an integer), unless otherwise indicated.

As used herein, the term “about” means±5% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

As used herein, the term “antigen” refers to an immunogenic molecule that provokes an immune response. The term “antigen” includes any protein, polypeptide, peptide, DNA, RNA, polynucleic acid, nucleic acid, or allergen that is capable of triggering an immune response in a subject. An antigen may be associated with a disease-causing agent, such as a bacterium, a virus, or a fungus, or it may be a protein or peptide that is capable of triggering an allergic or an autoimmune reaction in a subject.

As used herein, the terms “antibody” and “immunoglobulin” are interchangeable, and refer to the immunological proteins that are developed within a host subject's body or by tissue culture methods to have an affinity for a target antigen. An antibody or immunoglobulin is said to be “against” or to “bind” or “anti-” an antigen to which it has affinity.

As used herein, the term “control” refers to a reference standard. A positive control is known to provide a positive test result. A negative control is known to provide a negative test result.

As used herein, the term “detectably labelled” refers to particles or molecules having chemical or physical characteristics that permit the presence and/or quantity of the particles or molecules to be detected. Detectable labels include, but are not limited to, fluorescence properties, luminescent properties, and colorimetric properties.

Examples of labels having fluorescent properties are green fluorescent protein, fluorescein, and phycoerythrin.

As used herein, the term “identifiably labelled” refers to particles or molecules having chemical or physical characteristics that permit different species of particles or molecules to be distinguished. For example, identifiably labelled species of microparticles are species of microparticles wherein each individual species of microparticle can be distinguished from all other species of microparticle present in a mixture. Any appropriate type of identifiable label may be used, including size, magnetic properties, fluorescence properties and metal isotope properties. The identifiable label may be a property of the particle or molecule itself, or it may result from conjugation of a label to the particle or molecule.

As used herein, the term “microparticle” refers to particles having micrometer- or nanometer-scale cross-section dimensions. Microparticles may be of any shape, including spherical or approximately spherical. Microparticles are also be referred to as microparticles. Spherical or approximately spherical microparticles may be referred to as microspheres. Microparticles have a surface to which molecules may be attached. Such attached molecules are referred to as being conjugated to the microparticle. In some cases, microparticles have peptides or polypeptides on the surface that facilitate the conjugation of molecules to the surface of the microparticles. Type of molecules that may be conjugated to microparticles include, but are not limited to, polypeptides, proteins, and nucleic acids. Molecules conjugated to microparticles may be attached by any type of binding interaction, including but not limited to ionic bonding, hydrogen bonding, covalent bonding, Van der Waals bonding, and hydrophilic/hydrophobic interactions.

As used herein, the term “test sample” refers to a sample that is to be assayed for the presence of neutralizing antibodies. The test sample is a biological sample from a subject. Examples of test samples include, but are not limited to, whole blood, serum, plasma, nasal secretions, sputum, bronchial lavage, urine, stool, saliva, sweat, and cells that have membrane immunoglobulin (such as memory B cells).

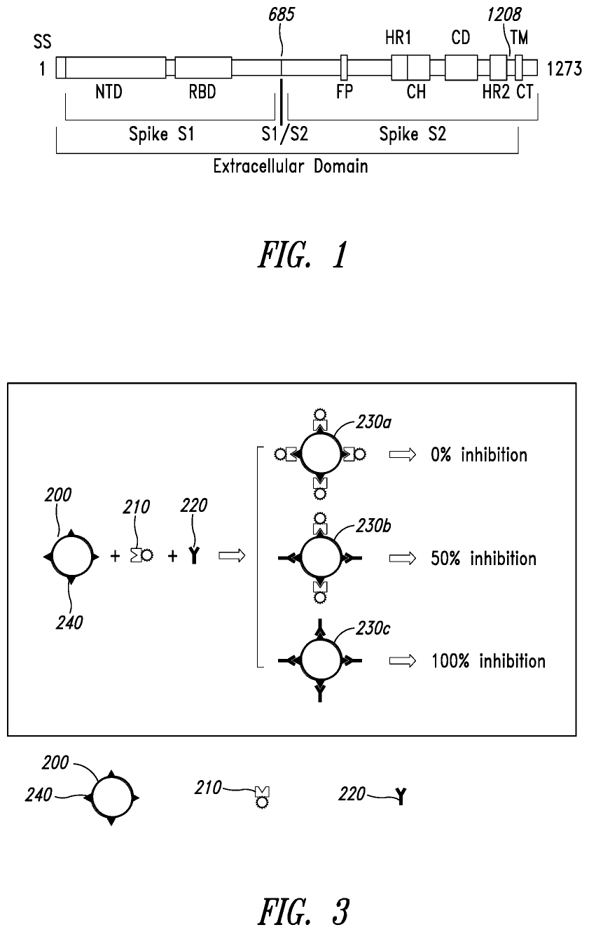

In some embodiments, the present disclosure provides assays that detect SARS-CoV-2 neutralizing antibodies in test samples by detecting antibodies that block the interaction of SARS-CoV-2 S protein with a SARS-CoV-2 S protein receptor. A schematic representation of full-length SARS-CoV-2 S protein is shown in FIG. 1. S protein comprises two subunits, S1 and S2, between which lies a protease cleavage site. The RBD, which is primarily responsible for binding of SARS-CoV-2 to the cell surface receptor ACE-2, is located within S1. In some embodiments, the SARS-CoV-2 S protein receptor is ACE-2.

The present disclosure refers to SARS-CoV-2 proteins and fragments thereof, in particular the spike (S) and nucleoprotein (NP) proteins and the S1 fragment (S1) or receptor binding domain (RBD) of the S protein, generically, in a manner that includes both the proteins from the originally isolated virus and variant proteins that have developed or will develop as the virus evolves into different variants. However, in some embodiments, the SARS-CoV-2 proteins and fragments thereof may also be limited to those from specific variants, depending on the type of neutralizing antibodies to be detected. For instance, an assay to specifically detect neutralizing antibodies for a variant of concern may contain SARS-CoV-2 variant proteins and fragments thereof specific to that viral variant.

Unless otherwise specifically defined, SARS-CoV-2 wild type and proteins or protein fragments thereof, such as S and RBD, are as described in Lan, J., Ge, J., Yu, J. et al. Structure of the SARS-CoV-2 spike receptor-binding domain bound to the ACE2 receptor. Nature 581, 215-220 (2020). https://doi.org/10.1038/s41586-020-2180-5.

Variants of SARS-CoV-2 included in the SARS-CoV-2 proteins and fragments thereof that may be assayed using a neutralizing assay of the present disclosure, and their associated S protein mutations, are as follows:

Wild-type—Wuhan-Hu-1—China—NCBI Reference Sequence: NC_045512.2 (Spike protein GeneID:43740568)
Beta—B.1.351—South Africa—K417N, E484K, N501Y, D614G, A701V
Gamma—P.1—Brazil—K417T, E484K, N501Y, D614G, H655Y
Delta—B.1.617.2 and AY lineages—India—L452R, T478K, D614G, P681R, particularly T19R, (V70F*), T95I, G142D, E156-, F157-, R158G, (A222V*), (W258L*), (K417N*), L452R, T478K, D614G, P681R, D950N
Mu—B.1.621—Colombia—R346K, E484K, N501Y, D614G, P681H
Lambda—C.37—Peru—L452Q, F490S, D614G
Omicron—B.1.1.529 and BA lineages—A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F
AY.4.2—United Kingdom—L452R, T478K, D614G, P681R, A222V, Y145H
C.36+L452R—Egypt—L452R, D614G, Q677H
B.1.1.318—E484K, D614G, P681H
P.1+P681H—Italy—D614G, E484K, H655Y, K417T, N501Y, P681H
B.1.617.2+K417N—United Kingdom—L452R, T478K, D614G, P681R, K417N
C.1.2—South Africa—D614G, E484K, H655Y, N501Y, N679K, Y449H
B.1.617.2+E484X (d)—India—L452R, T478K, D614G, P681R, E484X (d)
B.1.617.2+Q613H—India—L452R, T478K, D614G, P681R, Q613H
B.1.617.2+Q677H—India—L452R, T478K, D614G, P681R, Q677H
B.1.640—The Republic of Congo—D614G, F490R, N394S, N501Y, P681H, R346S, Y449N, 137-145del
Alpha—B.1.1.7—United Kingdom—N501Y, D614G, P681H
B.1.1.7+E484K—United Kingdom—E484K, N501Y, D614G, P681H
Epsilon—B.1.427/B.1.429—USA—L452R, D614G
B.1.616(c)—France—V483A, D614G, H655Y, G669S
B.1.525—Nigeria—E484K, D614G, Q677H
Theta—P.3—The Philippines—E484K, N501Y, D614G, P681H
Kappa—B.1.617.1—India—L452R, E484Q, D614G, P681R
B.1.620—S477N, E484K, D614G, P681H
B.1.617.3—India—L452R, E484Q, D614G, P681R
B.1.214.2—Q414K, N450K, ins214TDR, D614G
A.23.1+E484K—United Kingdom—V367F, E484K, Q613H
A.27—L452R, N501Y, A653V, H655Y
A.28—E484K, N501T, H655Y
C.16—L452R, D614G
B.1.351+P384—South Africa—P384L, K417N, E484K, N501Y, D614G, A701V
B.1.351+E516Q—K417N, E484K, N501Y, E516Q, D614G, A701V
B.1.1.7+L452R—United Kingdom—L452R, N501Y, D614G, P681H
B. 1.1.7+S494P—United Kingdom—S494P, N501Y, D614G, P681H
Iota—B.1.526—USA—E484K, D614G, A701V
B.1.526.1—USA—L452R, D614G
B.1.526.2—USA—S477N, D614G
Zeta—P.2—Brazil—E484K, D614G
B.1.1.519—Mexico—T478K, D614G
AV.1—United Kingdom—N439K, E484K, D614G, P681H
AT.1—Russia—E484K, D614G, N679K, ins679GIAL.

In some embodiments, the assays make use of multiple species of identifiably labelled microparticles, each species of microparticle being conjugated to a different SARS-CoV-2 protein or fragment thereof. The microparticles may be of any appropriate size and shape for use in the double-multiplex assay and may have micrometer- or nanometer-scale cross-section dimensions. Microparticles may also be referred to as beads. In certain embodiments, the microparticles have a cross-section that is from 0.001 μm to 1000 μm in length, 0.01 μm to 100 μm in length, 0.1 μm to 50 μm in length, 0.1 μm to 10 μm in length, 1 μm to 10 μm in length, 1 μm to 6 μm in length, 1 μm to 5 μm in length, or 1 μm to 3 μm in length. In certain embodiments, the microparticles are spherical or approximately spherical, in which case the cross-section may be a diametric cross-section and the microparticles may be referred to as microspheres. Microparticles have a surface to which molecules may be attached. Such attached molecules are referred to as being conjugated to the microparticle.

In some embodiments, the microparticles conjugated to a given SARS-CoV-2 protein or fragment thereof are identifiable, e.g. distinguishable from microparticles conjugated to a different SARS-CoV-2 protein or fragment thereof when two or more types of microparticles are used in the assay, or ascertainable by a detector when only one type of microparticle is used.

Microparticles may be distinguished by size, magnetic properties, fluorescence wavelength and/or intensity, ultraviolet-excited fluorescence wavelength and/or intensity violet-excited fluorescence wavelength and/or intensity, or any other appropriate property. Each distinguishable type of microparticle may have a surface upon which peptide or polypeptide residues are attached, enabling the binding of a protein or polypeptide. The protein or polypeptide may, in some embodiments, be attached to the surface of the microparticle or to a peptide or polypeptide residue on the surface of the microparticle by any type of binding interaction. Such binding interactions include, but are not limited to, ionic bonding, hydrogen bonding, covalent bonding, Van der Waals, and hydrophilic/hydrophobic interactions.

In certain embodiments, a SARS-CoV-2 S protein receptor or a SARS-CoV-2 S protein or fragment thereof is fluorescently labelled. Multiple fluorescent molecules appropriate for labelling of proteins and methods for attaching such molecules to proteins are known in the art. Any appropriate fluorescent molecule may be used. In some embodiments, the SARS-CoV-2 S protein receptor or SARS-CoV-2 S protein or fragment thereof is detectably labelled with phycoerythrin. In some embodiments, the SARS-CoV-2 S protein receptor or a SARS-CoV-2 S protein or fragment thereof is first biotinylated, and then combined with streptavidin-phycoerythrin, thereby forming fluorescently labelled SARS-CoV-2 S protein receptor or fluorescently labelled SARS-CoV-2 S protein or fragment thereof.

In some embodiments, the binding of fluorescently labelled SARS-CoV-2 S protein receptor or SARS-CoV-2 S protein or fragment thereof to the different species of microparticles is measured using flow cytometry.

In some embodiments, the test sample is whole blood, serum, plasma, interstitial fluid, nasal secretions, sputum, bronchial lavage, urine, stool, saliva, or sweat from a subject. In certain embodiments, the test sample is whole blood, serum, or plasma. The test sample may have a volume of 0.1 μl or more, such as a volume of 0.1-0.5 μl, 0.1-0.7 μl, 0.1-0.9 μl, 0.1-2.0 μL, 0.1-3.0 μL. 0.1-5.0 μL, 0.1-10.0 μL, 0.1-15.0 μL, or 0.1-20.0 μL. In some embodiments, the test sample volume is 0.1 μl, 0.2 μl, 0.3 μl, 0.4 μl, 0.5 μl, 0.6 μl, 0.7 μl, 0.8 μl, 0.9 μl, 1.0 μl, 1.1 μl, 1.2 μl, 1.3 μl, 1.4 μl, 1.5 μl, 1.6 μl, 1.7 μl, 1.8 μl, 1.9 μl, 2.0 μl, 2.1 μl, 2.2 μl, 2.3 μl, 2.4 μl, 2.5 μl, 2.6 μl, 2.7 μl, 2.8 μl, 2.9 μl, 3.0 μl, 3.1 μl, 3.2 μl, 3.3 μl, 3.4 μl, 3.5 μl, 3.6 μl, 3.7 μl, 3.8 μl, 3.9 μl, 4.0 μl, 4.1 μl, 4.2 μl, 4.3 μl, 4.4 μl, 4.5 μl, 4.6 μl, 4.7 μl, 4.8 μl, 4.9 μl, 5.0 μl, 5.5 μl, 10 μl, 10.5 μl, 11 μl, 11.5 μl, 12 μl, 12.5 μl, 13 μl, 13.5 μl, 14 μl, 14.5 μl, 15 μl, 15.5 μl, 16 μl, 16.5 μl, 17 μl, 17.5 μl, 18 μl, 18.5 μl, 19 μl, 19.5 μl, or 20 μl. The test sample may be used unaltered or components, such a stabilizing agent found in a collection vial, may be mixed with the test sample during the collection process. In instances where components are mixed with the test sample during the collection process, the test sample volume is the volume actually obtained from the subject, not the volume after mixing with components during the collection process. In such instances, the test sample volume may be estimated by subtracting any volume estimated to be contributed by components mixed with the sample during the collection process from the volume present after such mixing.

In some embodiments, the test sample is diluted before being assayed. For example, the test sample may be diluted 1:40, 1:30, 1:20, 1:10, 1:5, 1:2, or 1:1. Appropriate buffers for sample dilution are well known in the art. In some embodiments, the test sample is diluted in PBS buffer containing 1% bovine serum albumin (BSA). The test sample volume does not include any diluent volumes.

In some embodiments, the test sample may be assayed immediately, within about 5 minutes, within about 10 minutes within about 30 minutes, within about 60 minutes, within about 2 hours, within about 12 hours, within about 24 hours, within about 48 hours, or during a time interval between about any of these time points after collection of the test sample from the subject. Appropriate stabilization or preservative components may be added to the test sample, particularly if longer periods of time will elapse between collection and assay. Test samples may be frozen if needed.

Test samples may also result from processing of a sample as directly obtained from a patient. For example, if the test sample is plasma, it may be obtained by centrifuging a whole blood sample as directly obtained from a patient.

Test samples may be collected using any suitable methods and containers. For example, whole blood, serum, or plasma may be collected by venipuncture in a vacuum tube. Whole blood, serum, or plasma may also be collected by finger stick and a capillary action device. Whole blood, serum, plasma, or interstitial fluid may be collected using an alternative site stick, such as an arm stick as is commonly used in glucose monitoring, and a capillary action device. Samples secreted or expelled by the subject may simply be collected using standard laboratory processes and equipment. Bronchoalveolar lavage samples may be collected using a bronchoscope. In the limited instance of bronchoalveolar lavage, the test sample volume may include the fluid introduced into the airway in order to obtain the test sample.

In some embodiments, the detector used in an assay is a flow cytometer. For example, each type of identifiably labelled microparticle, if present, may be distinguished based on its distinguishing properties, and the proteins in a complex with a given type of identifiably labelled microparticle may be identified based on their detectable labels. In some embodiments, the microparticles are identifiably labelled by fluorescence properties and the detectably labelled protein(s) that may bind to the microparticles are fluorescently labelled, and the analysis is carried out using multi-color flow cytometry. In some embodiments, the microparticles are identifiably labelled by ultraviolet-excited or violet-excited fluorescence properties, the detectably labelled protein(s) are fluorescently labelled, and the analysis is carried out using multi-color flow cytometry.

In some embodiments, the microparticles are identifiably labelled by metal isotope and the detectably labelled protein(s) are metal isotope labelled, and the detector is a multi-metal isotope mass cytometer.

In some embodiments, the detector uses a mass cytometry method, such as CyTOF® (Fluidigm, California). CyTOF®, also known as cytometry by time of flight, is a technique based on inductively coupled plasma mass spectrometry and time of flight mass spectrometry. In this technique, isotopically pure elements, such as heavy metals, are conjugated to the detectably labelled protein(s). The unique mass signatures are then analyzed by a time of flight mass spectrometer.

In some embodiments, the assays described herein have any one or more of multiple advantages over other assay methods for neutralizing antibodies. One such advantage is that the assays closely mimic the interaction of S protein and ACE-2 that facilitates SARS-CoV-2 cell binding and entry, and the effect of neutralizing antibodies on the S protein/ACE-2 interaction. Another advantage is that the use of microparticles in combination with a flow cytometry or mass cytometry detection system provides excellent sensitivity and specificity. For example, some assays may be conducted with test samples having volumes of about 0.5 μl or less, which is significantly less material than is required for ELISA-based or cell-based assays. The very small sample volumes used in some assays of the present disclosure enable frequent, less invasive sample collection and facilitate adaptation of the assays for direct-to-consumer applications and sample collection in non-medical settings. Additionally, the assays are simple to use and can be completed in less than two hours, yet provide results that correlate with the more complicated and time-consuming cell-based assay that is currently the gold standard for detection of neutralizing antibodies. Such cell-based assays also must be carried out under restrictive safety procedures, as they use potentially infectious materials, whereas any infection risk of the present assays comes solely from the test samples themselves, such that the assays do not require safety precautions beyond those typically observed with human test samples. Finally, some assays may provide additional information that is not available from the alternative assays.

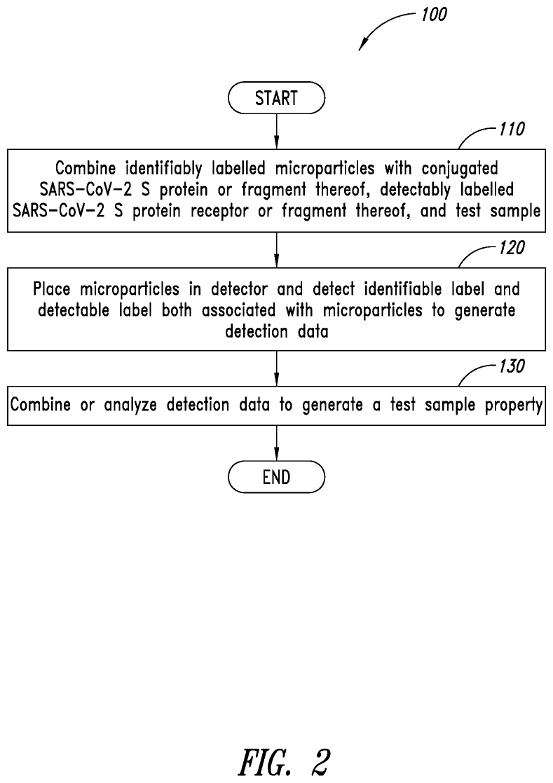

The above and other aspects of assays, compositions, and kits disclosed herein, including any aspects from the Examples 1-16, may be used in conjunction with the specific Platform 1-5 assays described in FIG. 2-FIG. 9 and and the specific Embodiments 1-176 described herein.

### Platform 1 Assay

The Platform 1 assay 100 of FIG. 2 detects neutralizing antibodies in a test sample.

In step 110, a test sample from a subject is combined with an identifiably labelled type of microparticle conjugated to a SARS-CoV-2 S protein or fragment thereof and also with a detectably labelled SARS-CoV-2 S protein receptor, such as human ACE-2 or a fragment thereof. Although step 110 is illustrated as a single combining step, in step 110, all three materials may be combined concurrently, or step 110 may occur in substeps, with the test sample first being combined with the identifiably labelled microparticle or the detectably labelled SARS-CoV-2 S protein receptor or fragment thereof, then later combined with the other material.

Regardless of the timing or order of combination of materials within step 110, the test sample, identifiably labelled microparticles, and detectably labelled SARS-CoV-2 S protein receptor or fragment thereof are combined under conditions and for a period of time sufficient to allow the detectably labelled SARS-CoV-2 S protein receptor or fragment thereof to bind to the SARS-CoV-2 S protein or fragment thereof on the identifiably labelled microparticles to form S protein-receptor complexes, if not prevented from doing so by a neutralizing antibody, and for neutralizing antibodies, if present in the test sample, to bind to the SARS-CoV-2 S protein or fragment thereof on the identifiably labelled microparticles to form S protein-neutralizing antibody complexes and, thereby, block the binding of the detectably labelled SARS-CoV-2 S protein receptor or fragment thereof to the identifiably labelled microparticles to form S protein-receptor complexes.

In effect, during step 110, any neutralizing antibodies in the test sample may compete with the detectably labelled SARS-CoV-2 S protein receptor or fragment thereof for binding to the SARS-CoV-2 S protein or fragment thereof. As a result, neutralizing antibodies reduce the amount of detectably labelled protein that becomes bound to the identifiably labelled microparticles during step 110. Step 110 may result in the formation of any of a variety of microparticle complexes, which may include S protein-receptor complexes, S protein-neutralizing antibody complexes, and hybrid complexes, which contain both S protein receptor and neutralizing antibodies bound to the SARS-CoV-2 S protein or fragment thereof conjugated to the identifiably labelled microparticles.

In some embodiments, the period of time of step 110 may be 1 minute, 2 minutes, 5 minutes, 10 minutes, 20 minutes, 30 minutes, 40 minutes, 50 minutes, 60 minutes or an interval between any of these times.

The identifiably labelled microparticles used in step 110 may include microparticles 200 illustrated in FIG. 3. In some embodiments, the SARS-CoV-2 S protein or fragment thereof 240 conjugated to the identifiably labelled microparticles may be conjugated before an identifiable label (not shown), and in some embodiments, the SARS-CoV-2 S protein or fragment thereof 240 may be conjugated after the identifiable label. In some embodiments, not shown, the microparticles may lack an identifiable label, as only one type of microparticle is used in Platform 1.

The detectably labelled SARS-CoV-2 S protein receptor or fragment thereof used in step 110 may include receptor 210 illustrated in FIG. 3. Neutralizing antibodies, if present, may include antibodies 220 illustrated in FIG. 3.

Furthermore, the microparticle complexes formed in step 110 may include microparticle complexes 230 illustrated in FIG. 3. An S protein-receptor complex 230a is illustrated, along with a S protein-neutralizing antibody complex 230c, and a hybrid complex 230b. In most assays, if a neutralizing antibody 220 is present in a test sample, the microparticle complexes 230 will include a combination of microparticle complexes 230a, 230b, and 230c, with hybrid complex 230b being most prevalent unless the neutralizing antibody 220 is particularly abundant in the test sample or binds with very high affinity, in which case S protein-neutralizing antibody complexes 230c may predominate, or unless the neutralizing antibody 220 is particularly scarce in the test sample or binds with very low affinity, in which case the S protein-receptor complexes 230a may predominate. If no neutralizing antibody 220 is present in the test sample, then only S protein-receptor complexes 230a may form in step 110.

Upon completion of step 110, in some embodiments, the microparticles are washed under conditions that do not substantially disrupt the complexes. For example, the microparticles may be washed with phosphate-buffered saline (PBS). This may remove unbound test sample components from the microparticles, which may then be placed in an appropriate liquid to maintain the complexes, such as additional PBS.

In step 120, the microparticles are placed in a detector that detects, for individual microparticle complexes, the microparticle type using the identifiable label (or simply microparticles if unlabeled microparticles are used), and the detectable label, and detection is performed. The presence or absence of or, more typically, the amount of detectable label associated with each microparticle complex may be collected or stored separately for each complex, or collected or stored in aggregate for all or a selected subset of microparticle complexes. Alternatively, or in addition, the type of microparticle complex may be detected and the number of each type of complex (i.e. S protein-receptor complex, S protein-neutralizing antibody complex, or hybrid complex) may be stored. Collection and storage in this context involves the use of a processor and memory in communication with part of the detector. Information generated by step 120 is referred to a detection data.

Positive and negative control samples may also be included in the assay (via performing a separate step 110 with the such samples or by virtue of the control samples being known microparticle complexes) and detected as appropriate in step 120 to provide additional detection data.

Detection data from the test sample may be referred to as sample detection data, while detection data from control samples may be referred to as control detection data. For example, total fluorescence intensity or mean fluorescence intensity, or both may be measured, as they correlate with the presence of neutralizing antibodies in the test sample.

In step 130, the detection data is combined or analyzed to generate a test sample property.

In some embodiments, the test sample property may simply be whether neutralizing antibodies are present in the test sample (e.g. positive or negative). This test sample property may be based on whether detectable label detected in the test sample in step 120 is below a set amount, a certain amount or proportion lower than a positive control containing abundant, high affinity neutralizing antibodies, a certain amount or proportion higher than a negative control containing antibodies, but not neutralizing antibodies (or, in some embodiments, simply containing no antibodies), or any combinations thereof.

In some embodiments, the test sample property may be more nuanced and provide information regarding the amount or affinity to neutralizing antibodies, or likely protective effects against infection with SARS-CoV-2 or moderate, severe, or critical illness if infected.

### Platform 2 Assay

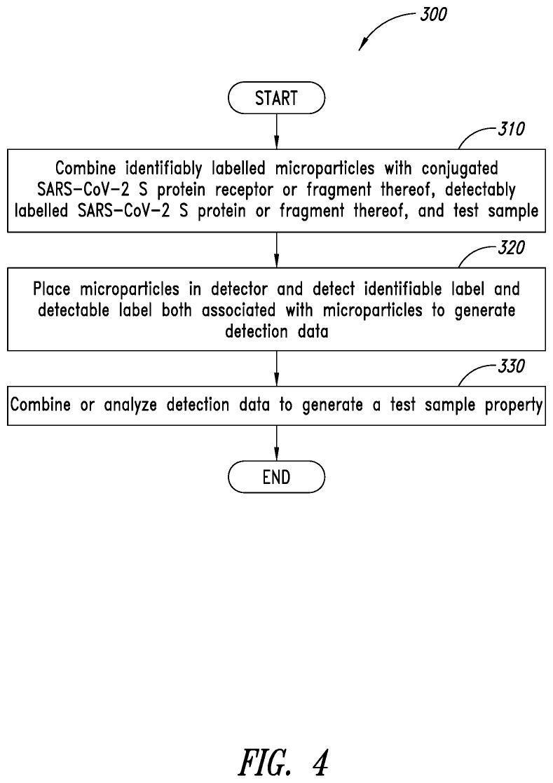

The Platform 2 assay 300 of FIG. 4 detects neutralizing antibodies in a test sample.

In step 310, a test sample from a subject is combined with an identifiably labelled type of microparticle conjugated to a SARS-CoV-2 S protein receptor, such as human ACE-2 or a fragment thereof and also with a detectably labelled SARS-CoV-2 S protein or fragment thereof. Although step 310 is illustrated as a single combining step, in step 310, all three materials may be combined concurrently, or step 310 may occur in substeps, with the test sample first being combined with the identifiably labelled microparticle or the detectably labelled SARS-CoV-2 S protein or fragment thereof, then later combined with the other material.

Regardless of the timing or order of combination of materials within step 310, the test sample, identifiably labelled microparticles, and detectably labelled SARS-CoV-2 S protein or fragment thereof are combined under conditions and for a period of time sufficient to allow the detectably labelled SARS-CoV-2 S protein or fragment thereof to bind to the SARS-CoV-2 S protein receptor or fragment thereof on the identifiably labelled microparticles to form receptor-S protein complexes, if not prevented from doing so by a neutralizing antibody, and for neutralizing antibodies, if present in the test sample, to bind to the detectably labelled SARS-CoV-2 S protein or fragment thereof to form neutralized S protein complexes and, thereby, block the binding of the detectably labelled SARS-CoV-2 S protein or fragment thereof to the identifiably labelled microparticles to form receptor-S protein complexes.

In effect, during step 310, any neutralizing antibodies in the test sample may compete with the SARS-CoV-2 S protein receptor or fragment thereof in identifiably microparticles for binding to the SARS-CoV-2 S protein or fragment thereof. As a result, neutralizing antibodies reduce the amount of detectably labelled protein that becomes bound to the identifiably labelled microparticles during step 310. Step 310 may result in the formation of any of a variety of microparticle complexes, which may include receptor-S-protein complexes and hybrid complexes, which contain both S protein and neutralizing antibodies bound to the SARS-CoV-2 S protein receptor or fragment thereof conjugated to the identifiably labelled microparticles. In step 310, neutralized S protein complexes, which are not associated with any microparticles, are also formed if neutralizing antibody is present, and may result in uncomplexed microparticles remaining.

In some embodiments, the period of time of step 310 may be 1 minute, 2 minutes, 5 minutes, 10 minutes, 20 minutes, 30 minutes, 40 minutes, 50 minutes, 60 minutes or an interval between any of these times.

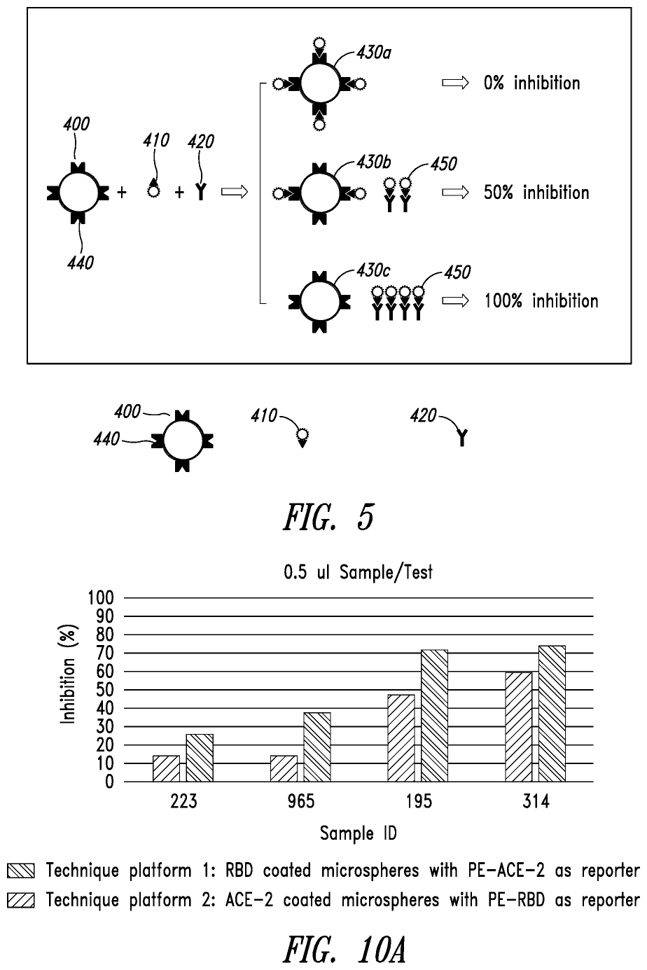

The identifiably labelled microparticles used in step 310 may include microparticles 400 illustrated in FIG. 5. In some embodiments, the SARS-CoV-2 S protein receptor or fragment thereof 440 conjugated to the identifiably labelled microparticles may be conjugated before an identifiable label (not shown), and in some embodiments, the SARS-CoV-2 S protein receptor or fragment thereof 440 may be conjugated after the identifiable label. In some embodiments, not shown, the microparticles may lack an identifiably label, as only one type of microparticle is used in Platform 2.

The detectably labelled SARS-CoV-2 S protein or fragment thereof used in step 310 may include S protein 410 illustrated in FIG. 5. Neutralizing antibodies, if present, may include antibodies 420 illustrated in FIG. 5.

Furthermore, the microparticle complexes formed in step 310 or existing after step 310 (in the case of uncomplexed microparticles) may include microparticle complexes 430 illustrated in FIG. 5. A receptor-S protein complex 430a is illustrated, along with a hybrid complex 430b, and a uncomplexed microparticle 430c. Neutralized S protein complexes 450 may also be formed in step 310. In most assays, if a neutralizing antibody 420 is present in a test sample, the microparticle complexes 430 will include a combination of microparticle complexes 430a and 430b and uncomplexed microparticles 430c, with hybrid complex 430b being most prevalent unless the neutralizing antibody 420 is particularly abundant in the test sample or binds with very high affinity, in which case uncomplexed microparticles 430c may predominate, or unless the neutralizing antibody 420 is particularly scarce in the test sample or binds with very low affinity, in which case the receptor-S protein complexes 430a may predominate. If no neutralizing antibody 420 is present in the test sample, then only receptor-S protein complexes 430a may form in step 310.

Upon completion of step 310, in some embodiments, the microparticles are washed under conditions that do not substantially disrupt the complexes, but that remove substantially all of the neutralized S protein complexes. For example, the microparticles may be washed with phosphate-buffered saline (PBS). This may remove unbound test sample components, including neutralized S protein complexes, from the microparticles, which may then be placed in an appropriate liquid to maintain the complexes, such as additional PBS.

In step 320, the microparticles are placed in a detector that detects, for individual microparticles, the microparticle type using the identifiable label (or simply microparticles if unlabeled microparticles are used), and the detectable label, and detection is performed. The presence or absence of or, more typically, the amount of detectable label associated with each microparticle may be collected or stored separately for each microparticle, or collected or stored in aggregate for all or a selected subset of microparticles. Alternatively, or in addition, the type of microparticle may be detected and the number of each type of microparticle complex (i.e. receptor-S protein complex or hybrid complex) or unbound microparticle may be stored. Collection and storage in this context involves the use of a processor and memory in communication with part of the detector. Information generated by step 320 is referred to a detection data.

Positive and negative control samples may also be included in the assay (via performing a separate step 310 with the such samples or by virtue of the control samples being known microparticle complexes) and detected as appropriate in step 320 to provide additional detection data.

Detection data from the test sample may be referred to as sample detection data, while detection data from control samples may be referred to as control detection data. For example, total fluorescence intensity may be measured, as it correlates with the presence of neutralizing antibodies in the test sample.

In step 330, the detection data is combined or analyzed to generate a test sample property.

In some embodiments, the test sample property may simply be whether neutralizing antibodies are present in the test sample (e.g. positive or negative). This test sample property may be based on whether detectable label detected in the test sample in step 320 is below a set amount, a certain amount or proportion lower than a positive control containing abundant, high affinity neutralizing antibodies, a certain amount or proportion higher than a negative control containing antibodies, but not neutralizing antibodies (or, in some embodiments, simply containing no antibodies), or any combinations thereof.

In some embodiments, the test sample property may be more nuanced and provide information regarding the amount or affinity to neutralizing antibodies, or likely protective effects against infection with SARS-CoV-2 or moderate, severe, or critical illness if infected.

### Platform 3 Assay

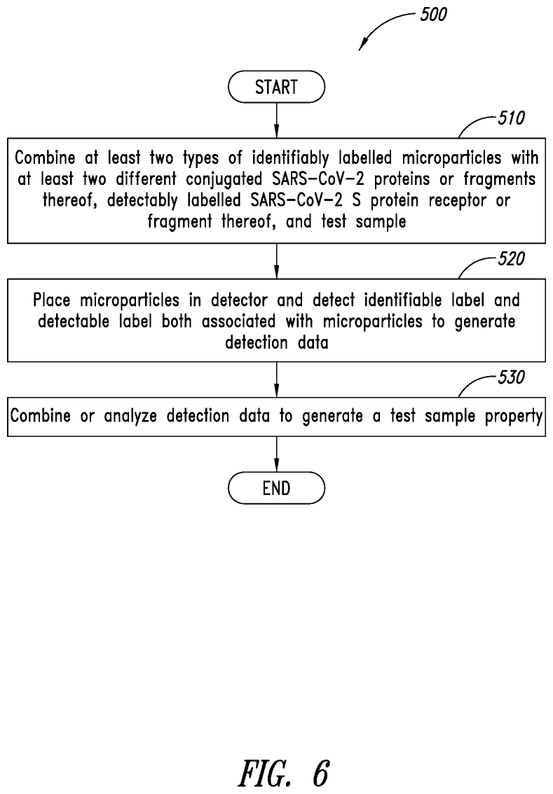

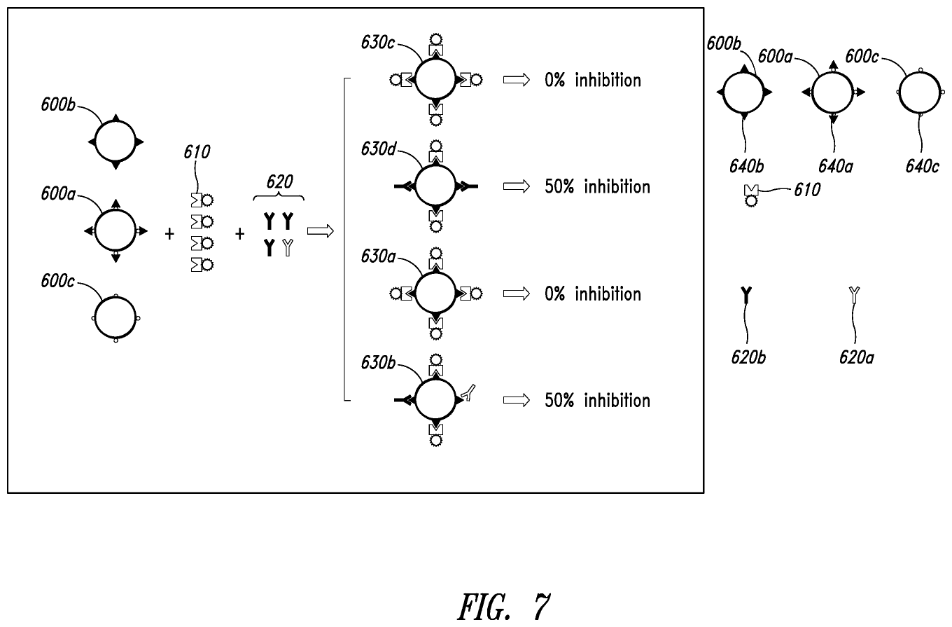

The Platform 3 assay 500 of FIG. 6 detects neutralizing antibodies in a test sample. Although Platform 3 is discussed in detail herein and illustrated in FIG. 6 and FIG. 7 with three types of microparticles, it may also be implemented with only two types of microparticles, lacking the third type of microparticle with conjugated third SARS-CoV-2 protein or fragment thereof, particularly NP. NP in this platform and other platforms and embodiments may serve as a control for a target antigen that does not bind the ACE-2 receptor.

In step 510, a test sample from a subject is combined with at least two types of identifiably labelled of microparticles, each conjugated to a different type of SARS-CoV-2 S protein or fragment thereof, and also with a detectably labelled SARS-CoV-2 S protein receptor, such as human ACE-2 or a fragment thereof. Although step 510 is illustrated as a single combining step, in step 510, all materials may be combined concurrently, or step 510 may occur in substeps, with the test sample first being combined with the identifiably labelled microparticle or the detectably labelled SARS-CoV-2 S protein receptor or fragment thereof, then later combined with the other material.

In some embodiments, a first type of identifiably labelled microparticle may be conjugated to a full-length SARS-CoV-2 S protein or a first fragment thereof, a second type of identifiably labelled microparticle may be conjugated to a second fragment of a SARS-CoV-2 S protein, such as the RBD, and a third type of identifiably labelled microparticle may be conjugated to a third SARS-CoV-2 protein or fragment thereof, such as NP.

Regardless of the timing or order of combination of materials within step 510, the test sample, identifiably labelled microparticles, and detectably labelled SARS-CoV-2 S protein receptor or fragment thereof are combined under conditions and for a period of time sufficient to allow the detectably labelled SARS-CoV-2 S protein receptor or fragment thereof to bind to the SARS-CoV-2 S protein or fragment thereof on the first type of identifiably labelled microparticles, to form S protein-receptor complexes and to bind the protein fragment on the second type of identifiably labelled microparticles to form protein fragment-receptor complexes, if not prevented from doing so by a neutralizing antibody, and for neutralizing antibodies, if present in the test sample, to bind to the SARS-CoV-2 S protein or fragment thereof on the first type of identifiably labelled microparticles to form S protein-neutralizing antibody complexes and to the protein fragment on the second type of identifiably labelled microparticles to form fragment-neutralizing antibody complexes, thereby, block the binding of the detectably labelled SARS-CoV-2 S protein receptor or fragment thereof to the first type of identifiably labelled microparticles to form S protein-receptor complexes and to the second type of identifiably labelled microparticles to form protein fragment-receptor complexes. Neither neutralizing antibodies nor SARS-CoV-2 S protein receptor of fragment thereof are expected to bind to the third type of microparticle, leaving uncomplexed microparticles of the third type.

In effect, during step 510, any neutralizing antibodies in the test sample may compete with the detectably labelled SARS-CoV-2 S protein receptor or fragment thereof for binding to the SARS-CoV-2 S protein or fragment thereof. As a result, neutralizing antibodies reduce the amount of detectably labelled protein that becomes bound to the identifiably labelled microparticles during step 510. Step 510 may result in the formation of any of a variety of microparticle complexes, which may include S protein-receptor complexes, S protein-neutralizing antibody complexes, and S protein hybrid complexes including the first type of identifiably labelled microsphere, protein fragment-receptor complexes, protein fragment-neutralizing antibody complexes, and protein fragment hybrid complexes including the second type of identifiably labelled microsphere, and, if non-specific binding of the detectably labelled SARS-CoV-2 S protein receptor or fragment thereof has occurred, nonspecific complexes including the third type of identifiably labelled microsphere. The third type of identifiably labelled microsphere should also remain as uncomplexed microparticles.

In some embodiments, the period of time of step 510 may be 1 minute, 2 minutes, 5 minutes, 10 minutes, 20 minutes, 30 minutes, 40 minutes, 50 minutes, 60 minutes or an interval between any of these times.

The identifiably labelled microparticles used in step 510 may include microparticles 600 illustrated in FIG. 7. A first type of identifiably labelled microparticle, 600a, includes a conjugated full-length SARS-CoV-2 S protein or a first fragment thereof, 640a. A second type of identifiably labelled microparticle, 600b, includes a conjugated second fragment of a SARS-CoV-2 S protein, RBD in the example illustrated, 640b. A third type of identifiably labelled microparticle, 600c, includes a conjugated third SARS-CoV-2 protein or fragment thereof, NP in the example illustrated, 640c.

In some embodiments, the SARS-CoV-2 S proteins or fragments thereof 640 conjugated to the identifiably labelled microparticles may be conjugated before an identifiable label (not shown), and in some embodiments, the SARS-CoV-2 S proteins or fragments thereof 640 may be conjugated after the identifiable label.

The detectably labelled SARS-CoV-2 S protein receptor or fragment thereof used in step 510 may include receptor 610 illustrated in FIG. 7. Neutralizing antibodies, if present, may include antibodies 620 illustrated in FIG. 7. Neutralizing antibodies 620b that bind to the second protein fragment, such as RBD, 640b, may be distinguished from neutralizing antibodies 620a that bind only to the full length S protein or first fragment thereof, 640a.

Furthermore, the microparticle complexes formed in step 510 may include microparticle complexes 630 illustrated in FIG. 7.

An S protein-receptor complex 630a is illustrated, a S protein-neutralizing antibody complex (not illustrated) may also be formed, as may S protein-hybrid complex 630b, which may include both types of neutralizing antibodies 620a and 620b, as illustrated, or only neutralizing antibody 620a (not shown), if neutralizing antibody 620b is able to bind to second protein fragment 640b, but not first protein or protein fragment 640a.

A protein fragment-receptor complex 640c is illustrated, a protein fragment-neutralizing antibody complex (not illustrated) may also be formed, as may protein fragment-hybrid complex 630d, which may include neutralizing antibody 620b, but not neutralizing antibody 620a.

In addition, uncomplexed microparticles (not shown) that result from the third type of microparticle 600c may be present. If non-specific S protein receptor or fragment thereof binding occurs, then complexes including microparticle 600c and detectably labelled S protein receptor of fragment thereof (not shown) may also be formed.

Upon completion of step 510, in some embodiments, the microparticles are washed under conditions that do not substantially disrupt the complexes. For example, the microparticles may be washed with phosphate-buffered saline (PBS). This may remove unbound test sample components from the microparticle complexes, which may then be placed in an appropriate liquid to maintain the complexes, such as additional PBS.

In step 520, the microparticles are placed in a detector that detects, for individual microparticle complexes or microparticles, the microparticle type by detecting the identifiable label and neutralizing antibody type by detecting the detectable label to generate detection data, and detection is performed. The identity of the identifiably labelled microparticle in each detected microparticle complex or microparticle as well as the presence or absence of or, more typically, the amount of neutralizing antibody against the protein or fragment thereof conjugated to the microparticle may be collected or stored separately for each complex, or collected or stored in aggregate based on identifiably labelled microparticle type. Alternatively or in addition, the identity of the neutralizing antibody in each detected microparticle complex as well as the presence or absence of or, more typically, the number of each type of identifiably labelled microparticle may be collected or stored separately for each microparticle complex or microparticle, or collected or stored in aggregate based on the conjugated protein or fragment thereof. Collection and storage in this context involves the use of a processor and memory in communication with part of the detector. Information generated by step 520 is referred to a detection data.

Positive and negative control samples may also be included in the assay (via performing a separate step 510 with the such samples or by virtue of the control samples being known microparticle complexes) and detected as appropriate in step 520 to provide additional detection data. The third type of microparticle 600c also serves as a control to detect non-specific binding of the detectably labelled SARS-CoV-2 S protein receptor or fragment thereof.

Detection data from the test sample may be referred to as sample detection data, while detection data from control samples may be referred to as control detection data. For example, total fluorescence intensity or mean fluorescence intensity, or both may be measured, as they correlate with the presence of neutralizing antibodies in the test sample.

In step 530, the detection data is combined or analyzed to generate a test sample property.

In some embodiments, the test sample property may simply be whether neutralizing antibodies are present in the test sample (e.g. positive or negative). This test sample property may be based on whether detectable label detected in the test sample in step 520 is below a set amount, a certain amount or proportion lower than a positive control containing abundant, high affinity neutralizing antibodies, a certain amount or proportion higher than a negative control containing antibodies, but not neutralizing antibodies (or, in some embodiments, simply containing no antibodies), or any combinations thereof.

In some embodiments, the test sample property may be more nuanced and provide information regarding the amount or affinity to neutralizing antibodies, or likely protective effects against infection with SARS-CoV-2 or moderate, severe, or critical illness if infected.

Measuring both neutralizing antibodies against both SARS-CoV-2 S1 protein and the RBD, specifically allows accurate detection of lower levels of neutralizing antibodies than is possible in assays that do not include both proteins. Specifically, in assays using only one protein, similar results may be obtained regardless of whether RBD or S protein, particularly S1, is used when detecting medium or high levels of neutralizing antibodies are present in the test sample. Low levels of neutralizing antibodies are typically seen in non-vaccinated individuals who may have had some exposure to viral antigens but not to the extent to cause a robust immune response.

### Platform 4 Assay

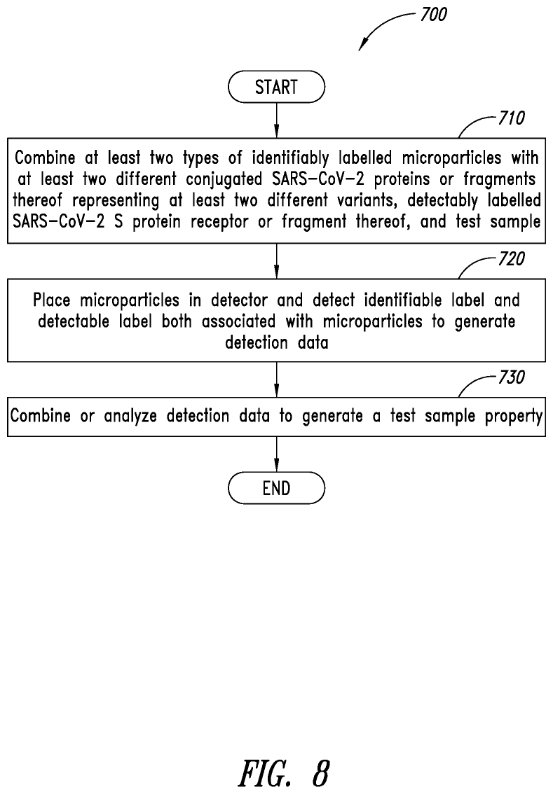

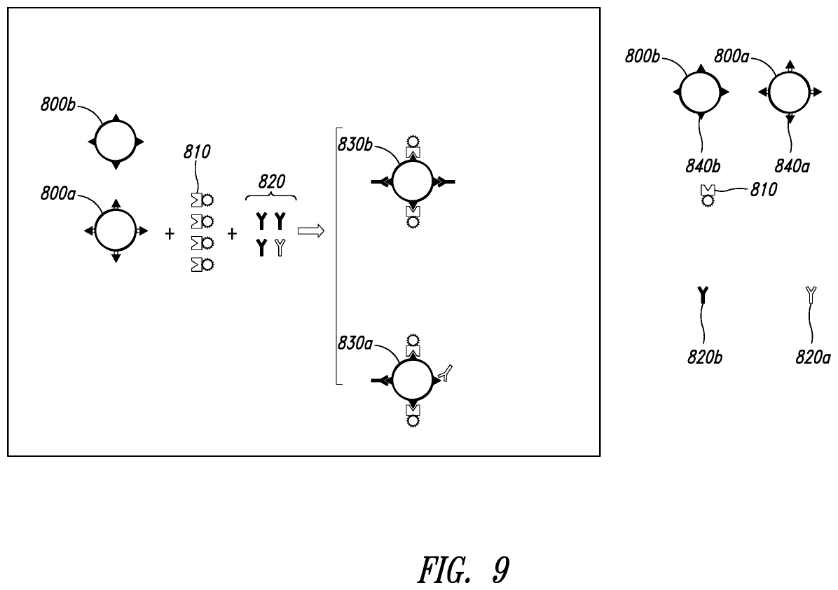

The Platform 4 assay 700 of FIG. 8 detects neutralizing antibodies in a test sample. Although Platform 4 is discussed in detail herein and illustrated in FIG. 8 and FIG. 9 with two types of microparticles, it may also be implemented with more than two microparticles, such as at least two, at least three, at least four, at least five, at least six, at least seven, at least eight, at least nine, at least ten, at least fifteen, at least twenty, or at least fifty types of microparticles. The total types of microparticles may be between any of the preceding values and twenty, fifty, or one hundred.

In step 710, a test sample from a subject is combined with at least two types of identifiably labelled of microparticles, each conjugated to a different type of SARS-CoV-2 proteins, typically S protein or RBD protein, or fragment thereof, representing at least two different variants of SARS-CoV-2, and also with a detectably labelled SARS-CoV-2 S protein receptor, such as human ACE-2 or a fragment thereof. Although step 710 is illustrated as a single combining step, in step 710, all materials may be combined concurrently, or step 710 may occur in substeps, with the test sample first being combined with the identifiably labelled microparticle or the detectably labelled SARS-CoV-2 S protein receptor or fragment thereof, then later combined with the other material. In addition, step 710 (and, optionally, also step 720) may be conducted using only one or a subset of the types of microparticles, with step 710 (and, optionally, also step 720) being duplicated for the other microparticles in sufficient iterations to perform assay 700 with all types of microparticles.

In some embodiments, a first type of identifiably labelled microparticle may be conjugated to a first SARS-CoV-2 S protein or RBD or fragment thereof derived from a first SARS-CoV-2 variant (which may be wild type) and a second type of identifiably labelled microparticle may be conjugated to a second SARS-CoV-2 S protein or RBD or fragment thereof derived from a second SARS-CoV-2 variant. In a specific embodiment, the type of SARS-CoV-2 protein for both types of microparticles is the same, e.g. both S protein or the same fragment type thereof, or both RBD or the same fragment type thereof. In another specific embodiment, a third type of identifiably labelled microparticle may be conjugated to wild type SARS-CoV-2 S protein. In another specific embodiment, third and fourth types of identifiably labelled microparticles may be conjugated separately to S protein or RBD or fragment thereof, whichever is not represented in the first and second types of identifiably labeled microparticles, from the same variants as the first and second types of identifiably labeled microparticles. In a specific embodiment, the type of SARS-CoV-2 protein for both the third and fourth types of microparticles is the same. For example, the first type of identifiably labeled microparticle may be conjugated to wild type S1 protein, the second type of identifiably labeled microparticle may be conjugated to the Omicron variant S1 protein, the third type of identifiably labeled microparticle may be conjugated to the wild type RBD, and the fourth type of identifiably labeled microparticle may be conjugated to the Omicron variant RBD. This scheme may be expanded for at least two, at least three, at least four, at least five, at least six, at least seven, at least eight, at least nine, at least ten, at least fifteen, at least twenty, or at least fifty variants (including wild type). The total types of variants may be between any of the preceding values and twenty, fifty, or one hundred. In some embodiments, yet another type of identifiably labelled microparticle is conjugated to SARS-CoV-2 NP.

Regardless of the timing or order of combination of materials within step 710, the test sample, identifiably labelled microparticles, and detectably labelled SARS-CoV-2 S protein receptor or fragment thereof are combined under conditions and for a period of time sufficient to allow the detectably labelled SARS-CoV-2 S protein receptor or fragment thereof to bind to the SARS-CoV-2 S protein or RBD or fragment thereof on the identifiably labelled microparticles, to form protein or fragment-receptor complexes, if not prevented from doing so by a neutralizing antibody, and for neutralizing antibodies, if present in the test sample, to bind to the SARS-CoV-2 S protein or RBD fragment thereof on the identifiably labelled microparticles to form protein or fragment-neutralizing antibody complexes, thereby, block the binding of the detectably labelled SARS-CoV-2 S protein receptor or fragment thereof to the identifiably labelled. Neither neutralizing antibodies nor SARS-CoV-2 S protein receptor of fragment thereof are expected to bind to the type of microparticle conjugated to NP (not show), leaving uncomplexed NP microparticles.

In effect, during step 710, any neutralizing antibodies in the test sample may compete with the detectably labelled SARS-CoV-2 S protein receptor or fragment thereof for binding to the SARS-CoV-2 S protein or RBD or fragment thereof. As a result, neutralizing antibodies reduce the amount of detectably labelled protein that becomes bound to the identifiably labelled microparticles during step 710. Step 710 may result in the formation of any of a variety of microparticle complexes, which may include protein or fragment-receptor complexes, protein or fragment-neutralizing antibody complexes, and protein or receptor hybrid complexes including type of identifiably labelled microspheres, and, if non-specific binding of the detectably labelled SARS-CoV-2 S protein receptor or fragment thereof has occurred, nonspecific complexes including the NP identifiably labelled microsphere.

In some embodiments, the period of time of step 710 may be 1 minute, 2 minutes, 5 minutes, 10 minutes, 20 minutes, 30 minutes, 40 minutes, 50 minutes, 60 minutes or an interval between any of these times.

The identifiably labelled microparticles used in step 710 may include microparticles 800 illustrated in FIG. 9. A first type of identifiably labelled microparticle, 800a, includes a conjugated SARS-CoV-2 S protein or RBD or fragment thereof from a first SARS-CoV-2 variant, 840a. A second type of identifiably labelled microparticle, 800b, includes a SARS-CoV-2 S protein or RBD or fragment thereof from a second SARS-CoV-2 variant, 840b. A third or subsequent type of identifiably labelled microparticle (not shown) may include a conjugated SARS-CoV-2 protein or fragment thereof from a third SARS-CoV-2 variant, or SARS-CoV-2 NP. Multiple additional types of identifiably labelled microparticles may be included to represent additional SARS-CoV-2 variants or both S protein or a fragment thereof and RBD or a fragment thereof from the same variant.

In some embodiments, the SARS-CoV-2 S proteins or RBDs or fragments thereof 840 conjugated to the identifiably labelled microparticles may be conjugated before an identifiable label (not shown), and in some embodiments, the SARS-CoV-2 S proteins or RBDs or fragments thereof 840 may be conjugated after the identifiable label.

The detectably labelled SARS-CoV-2 S protein receptor or fragment thereof used in step 710 may include receptor 810 illustrated in FIG. 9. Neutralizing antibodies, if present, may include antibodies 820 illustrated in FIG. 9. Neutralizing antibodies 820a that bind to the S protein or RBD or fragment thereof from the second SARS-CoV-2 variant, 840b, may be distinguished from neutralizing antibodies 820b that bind to the S protein or RBD or fragment thereof from both the first and second SARS-CoV-2 variants, 840a. Neutralizing antibodies (not shown) that bind to only the S protein or RBD or fragment thereof from the first SARS-CoV-2 variant may also be distinguished.

Furthermore, the microparticle complexes formed in step 710 may include microparticle complexes 830 illustrated in FIG. 9.

Protein or fragment-hybrid complexes 830a and 830b are illustrated. In protein or fragment-hybrid complex 830b, neutralizing antibodies 830b that can bind to the S protein or RBD or fragment thereof from both SARS-CoV-2 variants bind to the S protein or RBD or fragment thereof from the second SARS-CoV-2 variant. In protein or fragment-hybrid complex 830a, neutralizing antibodies 830a that can only bind to the S protein or RBD or fragment thereof of the first SARS-CoV-2 variant, as well as neutralizing antibodies 830b that can bind to the S protein or RBD or fragment thereof from both SARS-CoV-2 variants both bind to the S protein or RBD or fragment thereof from the first SARS-CoV-2 variant. In both complex 830a and 830b, some S protein receptor or fragment thereof is also able to bind to the microparticles.

The type and relative number of microparticle complexes formed with the different detectably labeled S proteins is indicative of the present of neutralizing antibodies for the variants represented, the affinity of such antibodies for each variant, and whether the neutralizing antibodies are cross-reactive, with affinities for multiple variants.

In other embodiments (not shown), uncomplexed microparticles that are conjugated to S protein or RBD or fragment thereof from a SARS-CoV-2 variant for which the sample has no neutralizing antibodies may also be present, as may uncomplexed microparticles that are conjugated to SARS-CoV-2 NP. In still other embodiments (not shown), in which two different neutralizing antibodies bind to only the first SARS-CoV-2 variant or only the second SARS-CoV-2 variant, only protein or fragment-hybrid complexes of the 830b type, with only one type of bound neutralizing antibody, are formed. In still other embodiments, in which SAR-CoV-2 S protein receptor-S protein or RBD or fragment thereof binding is nearly completely inhibited, protein fragment-neutralizing antibody complexes (not shown) may primarily be formed, at least with respect to one SARS-CoV-2 variant.

A protein fragment-receptor complex 640c is illustrated, a protein fragment-neutralizing antibody complex (not illustrated) may also be formed, as may protein

Upon completion of step 710, in some embodiments, the microparticles are washed under conditions that do not substantially disrupt the complexes. For example, the microparticles may be washed with phosphate-buffered saline (PBS). This may remove unbound test sample components from the microparticle complexes, which may then be placed in an appropriate liquid to maintain the complexes, such as additional PBS.

In step 720, the microparticles are placed in a detector that detects, for individual microparticle complexes or microparticles, the microparticle type by detecting the identifiable label and neutralizing antibody type by detecting the detectable label to generate detection data, and detection is performed. The identity of the identifiably labelled microparticle in each detected microparticle complex or microparticle as well as the presence or absence of or, more typically, the amount of neutralizing antibody against the protein or fragment thereof conjugated to the microparticle may be collected or stored separately for each complex, or collected or stored in aggregate based on identifiably labelled microparticle type. Alternatively or in addition, the identity of the neutralizing antibody in each detected microparticle complex as well as the presence or absence of or, more typically, the number of each type of identifiably labelled microparticle may be collected or stored separately for each microparticle complex or microparticle, or collected or stored in aggregate based on the conjugated protein or fragment thereof. Collection and storage in this context involves the use of a processor and memory in communication with part of the detector. Information generated by step 720 is referred to a detection data.

Positive and negative control samples may also be included in the assay (via performing a separate step 710 with the such samples or by virtue of the control samples being known microparticle complexes) and detected as appropriate in step 720 to provide additional detection data. The third type of microparticle with conjugated NP (not shown), if used, also serves as a control to detect non-specific binding of the detectably labelled SARS-CoV-2 S protein receptor or fragment thereof.

Detection data from the test sample may be referred to as sample detection data, while detection data from control samples may be referred to as control detection data. For example, total fluorescence intensity or mean fluorescence intensity, or both may be measured, as they correlate with the presence of neutralizing antibodies in the test sample.

In step 730, the detection data is combined or analyzed to generate a test sample property.

In some embodiments, the test sample property may simply be whether neutralizing antibodies are present in the test sample for each variant (e.g. positive or negative by variant). This test sample property may be based on whether detectable label detected in the test sample in step 820 is below a set amount for a type of microparticle conjugated to proteins from a given variant, a certain amount or proportion lower than a positive control containing abundant, high affinity neutralizing antibodies, a certain amount or proportion higher than a negative control containing antibodies, but not neutralizing antibodies (or, in some embodiments, simply containing no antibodies), or any combinations thereof.

In some embodiments, the test sample property may be more nuanced and provide information regarding the amount or affinity to neutralizing antibodies, or likely protective effects against infection with SARS-CoV-2 or moderate, severe, or critical illness if infected, or different levels of protection against different variants.

In some embodiments, measuring both neutralizing antibodies against both SARS-CoV-2 S1 protein and the RBD from multiple variants, specifically allows accurate detection of lower levels of neutralizing antibodies than is possible in assays that do not include both proteins. Specifically, in assays using only one protein, similar results may be obtained regardless of whether RBD or S protein, particularly S1, is used when detecting medium or high levels of neutralizing antibodies present in the test sample. Low levels of neutralizing antibodies are typically seen in non-vaccinated individuals who may have had some exposure to viral antigens but not to the extent to cause a robust immune response. In addition, low levels of neutralizing antibodies against a variant that are not protective against another variant may be seen in individuals who have had exposure to the first variant only.

In some embodiments, the test sample property or properties may be used to provide a diagnosis to patient.

### Platform 5 Assay

The Platform 5 assay (not illustrated) may correspond to the Platform 4 Assay in a manner similar to how the Platform 2 Assay corresponds to the Platform 1 Assay and detects neutralizing antibodies in a test sample. Although Platform 5 is described with reference to two types of microparticles, it may also be implemented with more than two microparticles, such as at least two, at least three, at least four, at least five, at least six, at least seven, at least eight, at least nine, at least ten, at least fifteen, at least twenty, or at least fifty types of microparticles. The total types of microparticles may be between any of the preceding values and twenty, fifty, or one hundred.

In a first step, a test sample from a subject is combined with an identifiably labelled type of microparticle conjugated to a SARS-CoV-2 S protein receptor, such as human ACE-2 or a fragment thereof and also with at least two different types of detectably labelled SARS-CoV-2 proteins, typically S protein or RBD protein, or a fragment thereof, representing at least two different variants of SARS-CoV-2. Although this step mar be performed as a single combining step, all three materials may be combined concurrently, or it may occur in substeps, with the test sample first being combined with the identifiably labelled microparticle or the detectably labelled SARS-CoV-2 S proteins or fragments thereof, then later combined with the other material.

Regardless of the timing or order of combination of materials within the first step, the test sample, identifiably labelled microparticles, and detectably labelled SARS-CoV-2 proteins or fragments thereof are combined under conditions and for a period of time sufficient to allow the detectably labelled SARS-CoV-2 proteins or fragments thereof to bind to the SARS-CoV-2 S protein receptor or fragment thereof on the identifiably labelled microparticles to form receptor-S protein complexes, if not prevented from doing so by a neutralizing antibody, and for neutralizing antibodies, if present in the test sample, to bind to the detectably labelled SARS-CoV-2 proteins or fragments thereof to form neutralized S protein complexes and, thereby, block the binding of the detectably labelled SARS-CoV-2 proteins or fragments thereof to the identifiably labelled microparticles to form receptor-S protein complexes.

In effect, during the first step, any neutralizing antibodies in the test sample may compete with the SARS-CoV-2 S protein receptor or fragment thereof in identifiably microparticles for binding to the SARS-CoV-2 proteins or fragments thereof. As a result, neutralizing antibodies reduce the amount of detectably labelled protein that becomes bound to the identifiably labelled microparticles. This first step may result in the formation of any of a variety of microparticle complexes, which may include receptor-S-protein complexes and hybrid complexes, which contain both S protein and neutralizing antibodies bound to the SARS-CoV-2 S protein receptor or fragment thereof conjugated to the identifiably labelled microparticles. Neutralized S protein complexes, which are not associated with any microparticles, are also formed if neutralizing antibody is present, and may result in uncomplexed microparticles remaining.

In some embodiments, the period of time of step 310 may be 1 minute, 2 minutes, 5 minutes, 10 minutes, 20 minutes, 30 minutes, 40 minutes, 50 minutes, 60 minutes or an interval between any of these times.

In some embodiments, the SARS-CoV-2 S protein receptor or fragment thereof conjugated to the identifiably labelled microparticles may be conjugated before an identifiable label (not shown), and in some embodiments, the SARS-CoV-2 S protein receptor or fragment thereof may be conjugated after the identifiable label.

In some embodiments, a first SARS-CoV-2 S protein may or RBD or fragment thereof derived from a first SARS-CoV-2 variant (which may be wild type) may have a first type of detectable label and a second SARS-CoV-2 S protein or RBD or fragment thereof derived from a second SARS-CoV-2 variant may have a second type of detectable label, which may be distinguishable from the first type of detectable label. In a specific embodiment, the SARS-CoV-2 proteins are of the same type, e.g. both S protein or the same fragment type thereof, or both RBD or the same fragment type thereof. In another specific embodiment, a third wild type SARS-CoV-2 S protein may have a third type of detectable label. In another specific embodiment, third and fourth SARS-CoV-2 S protein or RBD or fragment thereof, whichever is not represented in the first and second SARS-CoV-2 S protein, from the same variants as the first and second SARS-CoV-2 S protein may have third and fourth types of detectable labels. In a specific embodiment, the type of SARS-CoV-2 protein for both the third and fourth detectable labels is the same. For example, the first type of detectably labeled S protein may be wild type S1 protein, the second type of detectably labeled S protein may be the Omicron variant S1 protein, the third type of detectably labeled S protein may be wild type RBD, and the fourth type of detectably labeled S protein may be the Omicron variant RBD. This scheme may be expanded for at least two, at least three, at least four, at least five, at least six, at least seven, at least eight, at least nine, at least ten, at least fifteen, at least twenty, or at least fifty variants (including wild type). The total types of variants may be between any of the preceding values and twenty, fifty, or one hundred. In some embodiments SARS-CoV-2 NP is also detectably labeled and included in the assay.

If neutralizing antibodies for a variant are not present, then primarily receptor-S protein complexes with S proteins for that variant are formed. If neutralizing antibodies for a variant are present, then microparticles complexed with detectably labeled S protein from that variant will be limited, and hybrid complexes will be prevalent. The type and relative number of microparticle complexes formed with the different detectably labeled S proteins is indicative of the present of neutralizing antibodies for the variants represented, the affinity of such antibodies for each variant, and whether the neutralizing antibodies are cross-reactive, with affinities for multiple variants.

Upon completion of the first, in some embodiments, the microparticles are washed under conditions that do not substantially disrupt the complexes, but that remove substantially all of the neutralized S protein complexes. For example, the microparticles may be washed with phosphate-buffered saline (PBS). This may remove unbound test sample components, including neutralized S protein complexes, from the microparticles, which may then be placed in an appropriate liquid to maintain the complexes, such as additional PBS.

In a second step, the microparticles are placed in a detector that detects, for individual microparticles, the microparticle type using the identifiable label (or simply microparticles if unlabeled microparticles are used), and the detectable label, and detection is performed. The presence or absence of or, more typically, the amount of detectable label associated with each microparticle may be collected or stored separately for each microparticle, or collected or stored in aggregate for all or a selected subset of microparticles. Alternatively, or in addition, the type of microparticle may be detected and the number of each type of microparticle complex (i.e. receptor-S protein complex or hybrid complex) or unbound microparticle may be stored. Collection and storage in this context involves the use of a processor and memory in communication with part of the detector. Information generated by the second step is referred to a detection data.

Positive and negative control samples may also be included in the assay (via performing a separate first step with the such samples or by virtue of the control samples being known microparticle complexes) and detected as appropriate in the second step to provide additional detection data.

Detection data from the test sample may be referred to as sample detection data, while detection data from control samples may be referred to as control detection data. For example, total fluorescence intensity may be measured, as it correlates with the presence of neutralizing antibodies in the test sample.

In a third step, the detection data is combined or analyzed to generate a test sample property.

In some embodiments, the test sample property may simply be whether neutralizing antibodies are present in the test sample (e.g. positive or negative). This test sample property may be based on whether detectable label detected in the test sample in the second step is below a set amount, a certain amount or proportion lower than a positive control containing abundant, high affinity neutralizing antibodies, a certain amount or proportion higher than a negative control containing antibodies, but not neutralizing antibodies (or, in some embodiments, simply containing no antibodies), or any combinations thereof.

In some embodiments, the test sample property may be more nuanced and provide information regarding the amount or affinity to neutralizing antibodies, or likely protective effects against infection with SARS-CoV-2 or moderate, severe, or critical illness if infected.

In some embodiments, measuring both neutralizing antibodies against both SARS-CoV-2 S1 protein and the RBD from multiple variants, specifically allows accurate detection of lower levels of neutralizing antibodies than is possible in assays that do not include both proteins. Specifically, in assays using only one protein, similar results may be obtained regardless of whether RBD or S protein, particularly S1, is used when detecting medium or high levels of neutralizing antibodies present in the test sample. Low levels of neutralizing antibodies are typically seen in non-vaccinated individuals who may have had some exposure to viral antigens but not to the extent to cause a robust immune response. In addition, low levels of neutralizing antibodies against a variant that are not protective against another variant may be seen in individuals who have had exposure to the first variant only.

In some embodiments, the test sample property or properties may be used to provide a diagnosis to patient.

Khoury, D. S. et al. Neutralizing antibody levels are highly predictive of immune protection from symptomatic SARS-CoV-2 infection. Nature Medicine, https://doi.org/10.1038/s41591-021-01377-8 (2021) evaluated mean neutralizing antibody levels in several vaccinated patient studies and one convalesced patient study and ultimately normalized all values to the mean convalesced levels. A 90% reduction in disease incidence was reported at the mean convalesced neutralizing antibody levels. While the mean neutralizing antibody levels in the vaccinated cohorts were higher for certain vaccines (including mRNA vaccines), the additional level of protection was not significantly higher. A further analysis was conducted using the 50% protective titer that has been historically used in influenza studies. This analysis showed that the level of neutralizing antibody needed to reduce disease incidence by half is 20% of the mean neutralizing antibody levels of the convalesced level. In other words, mean convalesced neutralizing antibody levels provide a 90% reduction in disease incidence while neutralizing antibody levels equating to 20% of the mean convalesced levels provide a 50% reduction in disease incidence. These cut points provide meaningful thresholds for individual assessment of risk when making decisions regarding timing of boosters.

In some embodiments, two cut points for analysis of the detection data may be established in generation of a diagnosis. A first cut point may correlate with an expected 90% reduction in disease incidence in the patient who provided the test sample. This cut point may be based upon neutralizing antibody levels in convalesced individuals. A second cut point may correlate with an expected 50% reduction in disease incidence in the patient who provided the test sample. This second cut point bay be based upon detected neutralizing antibody levels in the test sample that are at least 20% of the mean neutralizing antibody levels detected using the same type of assay in convalesced individuals. These two cut points may be further used to stratify the test sample property of whether neutralizing antibodies for SARS-CoV-2 are present in the test sample into four levels, none, low, medium, and high.

Risk is another factor to consider and can be broadly categorized as intrinsic or extrinsic and that may play a role in a diagnosis or recommendation. Intrinsic risk factors include co-morbidities that have been well-defined since the start of the pandemic. These co-morbidities may include obesity, diabetes, high blood pressure, immune suppression and others. Extrinsic risk factors signify one's exposure risk mainly defined by lifestyle conditions that result in higher or lower potential exposure to SARS-CoV-2. These extrinsic risk factors may include work environment (e.g. working from home versus a hospital), travel frequency, as well as frequency and types of social interactions among others.

Collectively, knowing one's levels of NAb factored in with one's intrinsic and extrinsic risks can provide guidelines for decision making. To illustrate using a couple of scenarios, an individual with no known intrinsic risk factors who works from home and does not engage in many social activities in large crowds may be content with NAb levels above 20% of the convalesced levels; conversely, an individual with certain co-morbidities who is planning a cruise may elect to have a booster to elevate their NAb levels from >20% of convalesced levels to levels equaling to the mean convalesced NAb levels or higher.

Finally, determining the test sample property may include converting the amounts of neutralizing antibodies detected in an assay into a standard antibody measure, such as IU/mL. A specific formula may be used for each type of assay, based on historical assay results.

### Kits

In some embodiments, the present disclosure provides kits for detection of SARS-CoV-2 neutralizing antibodies. In certain embodiments, such kits may include a) a first type of identifiably labelled microparticle conjugated to a SARS-CoV-2 S protein or a first fragment thereof; b) a second type of identifiably labeled microparticle conjugated to a second fragment of a SARS-CoV-2 S protein; c) a third type of identifiably labelled microparticle conjugated to a SARS-CoV-2 nucleoprotein (NP) protein; and d) a detectably labelled SARS-CoV-2 S protein receptor or a fragment thereof. In some embodiments, the kit may alternatively contain microparticles and three different identifiably labels that the user may attach to the microparticles.

In some embodiments, the microparticles are identifiable by size, magnetic properties, fluorescence, ultraviolet-excited fluorescence wavelength, violet-excited fluorescence wavelength, fluorescence intensity, metal isotopes, or any combination thereof. In some embodiments, the first fragment of a SARS-CoV-2 S protein is or includes SARS-CoV-2 S protein S1. In some embodiments, the second fragment of a SARS-CoV-2 S protein is or includes SARS-CoV-2 S protein RBD. In some embodiments, the SARS-CoV-2 S protein receptor is ACE-2 or a fragment thereof. The SARS-CoV-2 S protein receptor may be fluorescently labelled, for example, the SARS-CoV-2 S protein receptor may be ACE-2 labelled with phycoerythrin. Any of the above-mentioned embodiments may further comprise a fourth species of microparticle conjugated to a full-length SARS-CoV-2 S protein.

In further embodiments, such kits may include a) identifiably labelled microparticles conjugated to a SARS-CoV-2 S protein or fragment thereof; and b) a detectably labelled SARS-CoV-2 S protein receptor or a fragment thereof. In some embodiments, the SARS-CoV-2 S protein or fragment thereof is or includes SARS-CoV-2 S protein S1 or SARS-CoV-2 S protein RBD. In some embodiments, the SARS-CoV-2 S protein receptor is ACE-2 or a fragment thereof. In some embodiments, the SARS-CoV-2 S protein receptor is ACE-2 or a fragment thereof. The SARS-CoV-2 S protein receptor may be fluorescently labelled, for example, the SARS-CoV-2 S protein receptor may be ACE-2 labelled with phycoerythrin.

In yet further embodiments, such kits may include: a) identifiably labelled microparticles conjugated to a SARS-CoV-2 S protein receptor or fragment thereof; and b) a detectably labelled SARS-CoV-2 S protein or a fragment thereof. In some embodiments, the SARS-CoV-2 S protein or fragment thereof is or includes SARS-CoV-2 S protein S1 or SARS-CoV-2 S protein RBD. In some embodiments, the SARS-CoV-2 S protein receptor is ACE-2 or a fragment thereof. The SARS-CoV-2 S protein or fragment thereof may be fluorescently labelled, for example, the SARS-CoV-2 S protein or fragment thereof may be SARS-CoV-2 S protein RBD labelled with phycoerythrin. In some embodiments, the SARS-CoV-2 S protein receptor is ACE-2 or a fragment thereof.

In another embodiment, the kit may include i) three types of identifiably labelled microparticles, a first type conjugated to a full-length SARS-CoV-2 S protein or a fragment thereof, particularly an S1 fragment, a second type conjugated to a fragment of a SARS-CoV-2 S protein, particularly an RBD, and a third type conjugated to a full-length SARS-CoV-2 NP or fragment thereof; and ii) a phycoerethrin-labelled human ACE-2 protein or a fragment thereof. The kit may optionally also contain a neutralizing antibody stain buffer, a neutralizing antibody stain, 1% BSA/PBS, PBS, or any combinations thereof.

In any of the above-mentioned kit embodiments, kits may further comprise positive and/or negative control samples, finger stick needles or blades, sample collection containers, supplies for returning a sample for analysis, such as a mailing kit or container appropriate for transport by courier, instructions for use, or any combination thereof.

The disclosure further provides compositions containing any combinations of materials used in the methods disclosed herein or provided in the kits disclosed herein.

The disclosure further provides the following embodiments:

Embodiment 1. A method of detecting SARS-CoV-2 neutralizing antibodies, the method comprising: a) combining at least two types of identifiably labelled microparticles conjugated to at least two different SARS-CoV-2 proteins or a fragment thereof, at least one of which comprises a SARS-CoV-2 S protein or fragment thereof, with a detectably labelled SARS-CoV-2 S protein receptor or a fragment thereof, and a test sample; b) detecting identifiable labels and the detectable label both associated with microparticles to generate detection data; c) combining or measuring the detection data to generate a test sample property relating to the presence or absence of or amount of neutralizing antibodies in the test sample.

Embodiment 2. The method of Embodiment 1, wherein the identifiably labelled microparticles include a first type of microparticle conjugated to a first fragment of SARS-CoV-2 S protein, a second type of microparticle conjugated to a second fragment of SARS-CoV-2 S protein, and a third type of microparticle conjugated to SARS-CoV-2 nuceloprotein (NP) protein or a fragment thereof.

Embodiment 3. The method of Embodiment 1, wherein the identifiably labelled microparticles include a first type of microparticle conjugated to a first fragment of SARS-CoV-2 S protein, a second type of microparticle conjugated to a second fragment of SARS-CoV-2 S protein, a third type of microparticle conjugated to SARS-CoV-2 nuceloprotein (NP) protein or a fragment thereof, and a fourth type of microparticle conjugated to a full-length SARS-CoV-2 S protein.

Embodiment 4. The method of any one of Embodiments 1-3, further comprising a wash step between steps a and b.

Embodiment 5. The method of any one of Embodiments 1-4, wherein the microparticles are microspheres.

Embodiment 6. The method of any one of Embodiments 1-5, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 7. The method of any one of Embodiments 2-6, wherein the SARS-CoV-2 S protein or fragment thereof is subunit 1 (S1) or a fragment thereof.

Embodiment 8. The method of Embodiment 7, wherein a first fragment of the SARS-CoV-2 S protein or fragment thereof is subunit 1 (S1) or a fragment thereof.

Embodiment 9. The method of any one of Embodiments 2-8, wherein the SARS-CoV-2 S protein or fragment thereof is receptor binding domain (RBD) or a fragment thereof.

Embodiment 10. The method of Embodiment 9, wherein a second fragment of SARS-CoV-2 S protein or fragment thereof is receptor binding domain (RBD) or a fragment thereof.

Embodiment 11. The method of any one of Embodiments 1-10, wherein the detectably labelled SARS-CoV-2 S protein receptor or fragment thereof is detectably labelled with a fluorescent molecule.

Embodiment 12. The method of Embodiment 11, wherein the fluorescent molecule is phycoerythrin.

Embodiment 13. The method of any one of Embodiments 1-10, wherein the detectably labelled SARS-CoV-2 S protein receptor or fragment thereof is biotinylated and is detected with a streptavidin-labelled fluorescent molecule.

Embodiment 14. The method of Embodiment 13, wherein the streptavidin-labelled fluorescent molecule is streptavidin-phycoerythrin.

Embodiment 15. The method of any one of Embodiments 1-14, wherein the SARS-CoV-2 S protein receptor or fragment thereof is human angiotensin-converting enzyme 2 (ACE-2) or a fragment thereof.

Embodiment 16. The method of any one of Embodiments 1-15, wherein the detecting step is carried out using flow cytometry or mass cytometry.

Embodiment 17. The method of any one of Embodiments 1-16, wherein the test sample is whole blood, serum, plasma, nasal secretions, sputum, bronchial lavage, urine, stool, or saliva.

Embodiment 18. The method of Embodiment 17, wherein the test sample is whole blood, serum, or plasma.

Embodiment 19. The method of Embodiment 18, wherein the whole blood, serum, or plasma is obtained by venipuncture or finger-stick.

Embodiment 20. The method of any one of Embodiments 17-19, wherein the test sample has a volume of 5 μl or less.

Embodiment 21. The method of any one of Embodiments 1-20, wherein the test sample is diluted prior to combining with the microparticles.

Embodiment 22. The method of any one of Embodiments 1-21, comprising using the test sample property to provide a diagnosis for a subject who provided the test sample.

Embodiment 23. The method of Embodiment 22, comprising providing a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies.

Embodiment 24. A method of detecting SARS-CoV-2 neutralizing antibodies, the method comprising: a) combining at least one identifiably labelled microparticle conjugated to a SARS-CoV-2 S protein or a fragment thereof and, optionally, a second identifiably labelled microparticle conjugated to another SARS-CoV-2 S protein or a fragment thereof or SARS-CoV-2 nucleoprotein (NP) or a fragment thereof, with a detectably labelled SARS-CoV-2 S protein receptor or a fragment thereof, and a test sample; b) detecting identifiable label and the detectable label both associated with microparticles to generate detection data; and c) combining or measuring the detection data to generate a test sample property relating to the presence or absence of or amount of neutralizing antibodies in the test sample

Embodiment 25. The method of Embodiment 24, further comprising a wash step between steps a and b.

Embodiment 26. The method of Embodiment 24 or 25, wherein the microparticles are microspheres.

Embodiment 27. The method of any one of Embodiments 24-26, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 28. The method of any one of Embodiments 24-27, wherein the SARS-CoV-2 S protein or fragment thereof is subunit 1 (S1) or a fragment thereof.

Embodiment 29. The method of any one of Embodiments 24-27, wherein the SARS-CoV-2 S protein or fragment thereof is receptor binding domain (RBD) or a fragment thereof.

Embodiment 30. The method of any one of Embodiments 24-29, further comprising combining identifiably labelled microparticles conjugated to a SARS-CoV-2 nucleoprotein (NP) or a fragment thereof and a test sample.

Embodiment 31. The method of any one of Embodiments 24-30, wherein the detectably labelled SARS-CoV-2 S protein receptor or fragment thereof is detectably labelled with a fluorescent molecule.

Embodiment 32. The method of Embodiment 31, wherein the fluorescent molecule is phycoerythrin.

Embodiment 33. The method of any one of Embodiments 24-30, wherein the detectably labelled SARS-CoV-2 S protein receptor or fragment thereof is biotinylated and is detected with a streptavidin-labelled fluorescent molecule.

Embodiment 34. The method of Embodiment 33, wherein the streptavidin-labelled fluorescent molecule is streptavidin-phycoerythrin.

Embodiment 35. The method of any one of Embodiments 24-34, wherein the SARS-CoV-2 S protein receptor or fragment thereof is human angiotensin-converting enzyme 2 (ACE-2) or a fragment thereof.

Embodiment 36. The method of any one of Embodiments 24-35, wherein the detecting step is carried out using flow cytometry or mass cytometry.

Embodiment 37. The method of any one of Embodiments 24-36, wherein the test sample is whole blood, serum, plasma, nasal secretions, sputum, bronchial lavage, urine, stool, or saliva.

Embodiment 38. The method of Embodiment 37, wherein the test sample is whole blood, serum, or plasma.

Embodiment 39. The method of Embodiment 38, wherein the whole blood, serum, or plasma is obtained by venipuncture or finger-stick.

Embodiment 40. The method of any one of Embodiments 37-39, wherein the test sample has a volume of 5 μl or less.

Embodiment 41. The method of any one of Embodiments 24-40, wherein the test sample is diluted prior to combining with the microparticles.

Embodiment 42. The method of any one of Embodiments 24-41, comprising using the test sample property to provide a diagnosis for a subject who provided the test sample.

Embodiment 43. The method of Embodiment 42, comprising providing a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies.

Embodiment 44. A method of detecting SARS-CoV-2 neutralizing antibodies, the method comprising: a) combining identifiably labelled microparticles conjugated to a SARS-CoV-2 S protein receptor or a fragment thereof with a detectably labelled SARS-CoV-2 S protein or a fragment thereof, and a test sample; b) detecting the identifiable label and the detectable label both associated with microparticles to generate detection data; c) combining or measuring the detection data to generate a test sample property relating to the presence or absence of or amount of neutralizing antibodies in the test sample.

Embodiment 45. The method of Embodiment 44, further comprising a wash step between steps a and b.

Embodiment 46. The method of Embodiment 44 or 45, wherein the microparticles are microspheres.

Embodiment 47. The method of any one of Embodiments 44-46, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 48. The method of any one of Embodiments 44-47, wherein the SARS-CoV-2 S protein or fragment thereof is subunit 1 (S1) or a fragment thereof.

Embodiment 49. The method of any one of Embodiments 44-47, wherein the SARS-CoV-2 S protein or fragment thereof is receptor binding domain (RBD) or a fragment thereof.

Embodiment 50. The method of any one of Embodiments 44-49, wherein the detectably labelled SARS-CoV-2 S protein or fragment thereof is detectably labelled with a fluorescent molecule.

Embodiment 51. The method of Embodiment 50, wherein the fluorescent molecule is phycoerythrin.

Embodiment 52. The method of any one of Embodiments 44-49, wherein the detectably labelled SARS-CoV-2 S protein or fragment thereof is biotinylated and is detected with a streptavidin-labelled fluorescent molecule.

Embodiment 53. The method of Embodiment 52, wherein the streptavidin-labelled fluorescent molecule is streptavidin-phycoerythrin.

Embodiment 54. The method of any one of Embodiments 44-53, wherein the SARS-CoV-2 S protein receptor or fragment thereof is human angiotensin-converting enzyme 2 (ACE-2) or a fragment thereof.

Embodiment 55. The method of any one of Embodiments 44-54, wherein the detecting step is carried out using flow cytometry or mass cytometry.

Embodiment 56. The method of any one of Embodiments 44-55, wherein the test sample is whole blood, serum, plasma, nasal secretions, sputum, bronchial lavage, urine, stool, or saliva.

Embodiment 57. The method of Embodiment 56, wherein the test sample is whole blood, serum, or plasma.

Embodiment 58. The method of Embodiment 57, wherein the whole blood, serum, or plasma is obtained by venipuncture or finger-stick.

Embodiment 59. The method of any one of Embodiments 56-58, wherein the test sample has a volume of 5 μl or less.

Embodiment 60. The method of any one of Embodiments 44-59, wherein the test sample is diluted prior to combining with the microparticles.

Embodiment 61. The method of any one of Embodiments 44-60, comprising using the test sample property to provide a diagnosis for a subject who provided the test sample.

Embodiment 62. The method of Embodiment 61, comprising providing a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies.

Embodiment 63. A method of detecting SARS-CoV-2 neutralizing antibodies for at least two SARS-CoV-2 variants, the method comprising: a) combining at least two types of identifiably labelled microparticles conjugated to at least two different SARS-CoV-2 S proteins, RBDs or fragment thereof from at least two different SARS-CoV-2 variants with a detectably labelled SARS-CoV-2 S protein receptor or a fragment thereof, and a test sample; b) detecting identifiable labels and the detectable label both associated with microparticles to generate detection data; c) combining or measuring the detection data to generate a test sample property relating to the presence or absence of or amount of neutralizing antibodies for both variants in the test sample.

Embodiment 64. The method of Embodiment 63, wherein the at least two different SARS-CoV-2 S proteins, RBDs or fragment thereof are both the same type of protein or fragment thereof from the two different SARS-CoV-2 variants.

Embodiment 65. The method of Embodiment 63 or 64, wherein the identifiably labelled microparticles further include an additional type of microparticle conjugated to SARS-CoV-2 nuceloprotein (NP) protein or a fragment thereof.

Embodiment 66. The method of any one of Embodiments 63-65, further comprising a wash step between steps a and b.

Embodiment 67. The method of any one of Embodiments 63-66, wherein the microparticles are microspheres.

Embodiment 68. The method of any one of Embodiments 63-67, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 69. The method of any one of Embodiments 63-68, wherein the SARS-CoV-2 S protein or fragment thereof is subunit 1 (S1) or a fragment thereof.

Embodiment 70. The method of Embodiment 69, wherein a first fragment of the SARS-CoV-2 S protein or fragment thereof is subunit 1 (S1) or a fragment thereof.

Embodiment 71. The method of any one of Embodiments 63-70, wherein the SARS-CoV-2 S protein or fragment thereof is receptor binding domain (RBD) or a fragment thereof.

Embodiment 72. The method of Embodiment 71, wherein a second fragment of SARS-CoV-2 S protein or fragment thereof is receptor binding domain (RBD) or a fragment thereof.

Embodiment 73. The method of any one of Embodiments 63-72, wherein the detectably labelled SARS-CoV-2 S protein receptor or fragment thereof is detectably labelled with a fluorescent molecule.

Embodiment 74. The method of Embodiment 73, wherein the fluorescent molecule is phycoerythrin.

Embodiment 75. The method of any one of Embodiments 63-72, wherein the detectably labelled SARS-CoV-2 S protein receptor or fragment thereof is biotinylated and is detected with a streptavidin-labelled fluorescent molecule.

Embodiment 76. The method of Embodiment 75, wherein the streptavidin-labelled fluorescent molecule is streptavidin-phycoerythrin.

Embodiment 77. The method of any one of Embodiments 63-76, wherein the SARS-CoV-2 S protein receptor or fragment thereof is human angiotensin-converting enzyme 2 (ACE-2) or a fragment thereof.

Embodiment 78. The method of any one of Embodiments 63-77, wherein the detecting step is carried out using flow cytometry or mass cytometry.

Embodiment 79. The method of any one of Embodiments 63-78, wherein the test sample is whole blood, serum, plasma, nasal secretions, sputum, bronchial lavage, urine, stool, or saliva.

Embodiment 80. The method of Embodiment 79, wherein the test sample is whole blood, serum, or plasma.

Embodiment 81. The method of Embodiment 80, wherein the whole blood, serum, or plasma is obtained by venipuncture or finger-stick.

Embodiment 82. The method of any one of Embodiments 79-81, wherein the test sample has a volume of 5 μl or less.

Embodiment 83. The method of any one of Embodiments 63-82, wherein the test sample is diluted prior to combining with the microparticles.

Embodiment 84. The method of any one of Embodiments 63-84, comprising using the test sample property to provide a diagnosis for a subject who provided the test sample.

Embodiment 85. The method of Embodiment 84, comprising providing a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies for each variant of SARS-CoV-2 tested.

Embodiment 86. A method of detecting SARS-CoV-2 neutralizing antibodies for at least two SARS-CoV-2 variants, the method comprising: a) combining identifiably labelled microparticles conjugated to a SARS-CoV-2 S protein receptor or a fragment thereof with at least two different detectably labelled SARS-CoV-2 S proteins, RBDs or fragment thereof from at least two different SARS-CoV-2 variants, and a test sample; b) detecting the identifiable label and the detectable labels both associated with microparticles to generate detection data; and c) combining or measuring the detection data to generate a test sample property relating to the presence or absence of or amount of neutralizing antibodies in the test sample.

Embodiment 87. The method of Embodiment 86, further comprising a wash step between steps a and b.

Embodiment 88. The method of Embodiment 86 or 87, wherein the microparticles are microspheres.

Embodiment 89. The method of any one of Embodiments 86-88, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 90. The method of any one of Embodiments 86-89, wherein the SARS-CoV-2 S proteins, RBDs, or fragment thereof is subunit 1 (S1) or a fragment thereof or receptor binding domain (RBD) or a fragment thereof.

Embodiment 91. The method of any one of Embodiments 86-90, wherein the SARS-CoV-2 S protein or fragment thereof is receptor binding domain (RBD) or a fragment thereof.

Embodiment 92. The method of any one of Embodiments 86-91, wherein the detectably labelled SARS-CoV-2 S proteins or fragment thereof are detectably labelled with a fluorescent molecule.

Embodiment 93. The method of Embodiment 92, wherein the fluorescent molecule is phycoerythrin.

Embodiment 94. The method of any one of Embodiments 86-91, wherein the detectably labelled SARS-CoV-2 S protein or fragment thereof is biotinylated and is detected with a streptavidin-labelled fluorescent molecule.

Embodiment 95. The method of Embodiment 94, wherein the streptavidin-labelled fluorescent molecule is streptavidin-phycoerythrin.

Embodiment 96. The method of any one of Embodiments 86-95, wherein the SARS-CoV-2 S protein receptor or fragment thereof is human angiotensin-converting enzyme 2 (ACE-2) or a fragment thereof.

Embodiment 97. The method of any one of Embodiments 86-96, wherein the detecting step is carried out using flow cytometry or mass cytometry.

Embodiment 98. The method of any one of Embodiments 86-97, wherein the test sample is whole blood, serum, plasma, nasal secretions, sputum, bronchial lavage, urine, stool, or saliva.

Embodiment 99. The method of Embodiment 98, wherein the test sample is whole blood, serum, or plasma.

Embodiment 100. The method of Embodiment 99, wherein the whole blood, serum, or plasma is obtained by venipuncture or finger-stick.

Embodiment 101. The method of any one of Embodiments 86-100, wherein the test sample has a volume of 5 μl or less.

Embodiment 102. The method of any one of Embodiments 86-101, wherein the test sample is diluted prior to combining with the microparticles.

Embodiment 103. The method of any one of Embodiments 86-102, comprising using the test sample property to provide a diagnosis for a subject who provided the test sample.

Embodiment 104. The method of Embodiment 103, comprising providing a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies.

Embodiment 105. A kit for detecting SARS-CoV-2 antibodies, the kit comprising: a first type of identifiably labelled microparticle conjugated to a SARS-CoV-2 S protein or a fragment thereof; a detectably labelled SARS-CoV-2 S protein receptor or a fragment thereof; and instructions for use.

Embodiment 106. The kit of Embodiment 105, further comprising a second type of identifiably labelled microparticle conjugated to a SARS-CoV-2 nucleoprotein (NP) protein.

Embodiment 107. The kit of Embodiment 105 or 106, further comprising: a) a first type of identifiably labelled microparticle conjugated to a SARS-CoV-2 S protein or a first fragment thereof; b) a second type of identifiably labeled microparticle conjugated to a second fragment of a SARS-CoV-2 S protein, which is different from the first fragment; and c) a third type of identifiably labelled microparticle conjugated to a NP protein.

Embodiment 108. The kit of any one of Embodiments 105-107, further comprising a second type of identifiably labelled microparticle conjugated to a SARS-CoV-2 S protein or a fragment thereof, wherein the SARS-CoV-2 S protein or a fragment thereof conjugated to the first type of identifiably labelled microparticle is from a first SARS-CoV-2 variant and the SARS-CoV-2 S protein or a fragment thereof conjugated to the second type of identifiably labelled microparticle is from a second SARS-CoV-2 variant.

Embodiment 109. The kit of Embodiment 108, further comprising at least one additional type of identifiably labelled microparticle conjugated to a SARS-CoV-2 S protein or a fragment thereof from at least one variant of SARS-CoV-2 that is different from the first SARS-CoV-2 variant and the second SARS-CoV-2 variant.

Embodiment 110, The kit of Embodiment 108 or 109, wherein the same SARS-CoV-2 S protein or a fragment thereof is the same type or protein or fragment from different variants of SARS-CoV-2.

Embodiment 111. The kit of any one of Embodiments 105-110, further comprising: a detectably labelled full-length SARS-CoV-2 S protein.

Embodiment 112. The kit of any one of Embodiments 105-111, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 113. The kit of any one of Embodiments 105-112, wherein the SARS-CoV-2 S protein or a fragment thereof is subunit 1 (S1) or a fragment thereof.

Embodiment 114. The kit of any one of Embodiments 105-112, wherein the SARS-CoV-2 S protein or fragment thereof is receptor binding domain (RBD) or a fragment thereof.

Embodiment 115. The kit of any one of Embodiments 105-114, wherein the SARS-CoV-2 S protein receptor or fragment thereof is human angiotensin-converting enzyme 2 (ACE-2) or a fragment thereof.

Embodiment 116. The kit of any one of Embodiments 105-115, wherein the detectably labelled SARS-CoV-2 S protein receptor or fragment thereof is detectably labelled with a fluorescent molecule.

Embodiment 117. The kit of Embodiment 116, wherein the fluorescent molecule is phycoerythrin.

Embodiment 118. The kit of any one of Embodiments 105-117, wherein the detectably labelled SARS-CoV-2 S protein receptor or fragment thereof is biotinylated and is detected with a streptavidin-labelled fluorescent molecule.

Embodiment 119. The kit of Embodiment 118, wherein the streptavidin-labelled fluorescent molecule is streptavidin-phycoerythrin.

Embodiment 120. The kit of any one of Embodiments 105-119, further comprising a neutralizing antibody stain buffer, a neutralizing antibody stain, 1% BSA/PBS, PBS, or any combinations thereof.

Embodiment 121. The kit of any one of Embodiments 105-120, further comprising a positive control sample, a negative control sample, a finger stick needle or blade, a sample collection container, supplies for returning a sample for analysis, or any combination thereof.

Embodiment 122. A kit for detecting SARS-CoV-2 antibodies, the kit comprising: an identifiably labelled microparticle conjugated to a SARS-CoV-2 S protein receptor or a fragment thereof; a detectably labelled SARS-CoV-2 S protein or a fragment thereof; and instructions for use.

Embodiment 123. The kit of Embodiment 122, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 124. The kit of Embodiment 122 or 123, wherein the SARS-CoV-2 S protein or a fragment thereof is subunit 1 (S1) or a fragment thereof.

Embodiment 125. The kit of Embodiment 122 or 123, wherein the SARS-CoV-2 S protein or fragment thereof is receptor binding domain (RBD) or a fragment thereof.

Embodiment 126. The kit of Embodiment 124 or 125, wherein the kit comprises two detectably labelled SARS-CoV-2 S proteins, RBDs, or fragments thereof from two different SARS-CoV-2 variants.

Embodiment 127. The kit of any one of Embodiments 122-126, wherein the SARS-CoV-2 S protein receptor or fragment thereof is human angiotensin-converting enzyme 2 (ACE-2) or a fragment thereof.

Embodiment 128. The kit of any one of Embodiments 122-127, wherein the detectably labelled SARS-CoV-2 S protein receptor or fragment thereof is detectably labelled with a fluorescent molecule.

Embodiment 129. The kit of Embodiment 128, wherein the fluorescent molecule is phycoerythrin.

Embodiment 130. The kit of any one of Embodiments 122-127, wherein the detectably labelled SARS-CoV-2 S protein receptor or fragment thereof is biotinylated and is detected with a streptavidin-labelled fluorescent molecule.

Embodiment 131. The kit of Embodiment 130, wherein the streptavidin-labelled fluorescent molecule is streptavidin-phycoerythrin.

Embodiment 132. The kit of any one of Embodiments 122-131, further comprising a neutralizing antibody stain buffer, a neutralizing antibody stain, 1% BSA/PBS, PBS, or any combinations thereof.

Embodiment 133. The kit of any one of Embodiments 122-132, further comprising a positive control sample, a negative control sample, a finger stick needle or blade, a sample collection container, supplies for returning a sample for analysis, or any combination thereof.

Embodiment 134. A composition comprising a mixture of at least two types of identifiable microparticles, a first type conjugated to a first SARS-CoV-2 S protein or fragment thereof, and a second type conjugated to a second fragment of SARS-CoV-2 S protein, which is different from the first fragment or to a second SARS-CoV-2 S protein from a different variant of SARS-CoV-2 than the first SARS-CoV-2 S protein.

Embodiment 135. The composition of Embodiment 134, further comprising a third type of identifiable microparticle conjugated to a third SARS-CoV-2 nucleoprotein (NP) or a fragment thereof.

Embodiment 136. The composition of Embodiment 134 or 135, further comprising an additional type of identifiable microparticle conjugated to a full-length SARS-CoV-2 S protein.

Embodiment 137. The composition of any one of Embodiments 134-136, further comprising at least one additional microparticle conjugated to a SARS-CoV-2 S protein or a fragment thereof from at least one variant of SARS-CoV-2 that is different from the SARS-CoV-2 variants whose proteins are conjugated to the first and second microspartibles.

Embodiment 138, The composition of Embodiment 134 or 137, wherein the same SARS-CoV-2 S protein or a fragment thereof is the same type or protein or fragment from different variants of SARS-CoV-2.

Embodiment 139. The composition of any one of Embodiments 134-138, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 140. The composition of any one of Embodiments 134-139, wherein the first SARS-CoV-2 S protein or a fragment thereof or the second fragment of SARS-CoV-2 S protein or second SARS-CoV-2 S protein from a different variant of SARS-CoV-2 than the first SARS-CoV-2 S protein is subunit 1 (S1) or a fragment thereof.

Embodiment 141. The composition of any one of Embodiments 134-140, wherein the first SARS-CoV-2 S protein or a fragment thereof or the second fragment of SARS-CoV-2 S protein is receptor binding domain (RBD) or a fragment thereof.

Embodiment 142. The composition of any one of Embodiments 134-141, further comprising a detectably labelled SARS-CoV-2 S protein receptor of fragment thereof.

Embodiment 143. The composition of Embodiment 142, wherein the SARS-CoV-2 S protein receptor or fragment thereof is human angiotensin-converting enzyme 2 (ACE-2) or a fragment thereof.

Embodiment 144. The composition of Embodiment 142 or 143, wherein the detectably labelled SARS-CoV-2 S protein receptor or fragment thereof is detectably labelled with a fluorescent molecule.

Embodiment 145. The composition of Embodiment 144, wherein the fluorescent molecule is phycoerythrin.

Embodiment 146. The composition of Embodiment 142 or 143, wherein the detectably labelled SARS-CoV-2 S protein receptor or fragment thereof is biotinylated and is detected with a streptavidin-labelled fluorescent molecule.

Embodiment 147. The composition of Embodiment 146, wherein the streptavidin-labelled fluorescent molecule is streptavidin-phycoerythrin.

Embodiment 148. The composition of any one of Embodiments 134-147, further comprising a SARS-CoV-2 neutralizing antibody.

Embodiment 149. The composition of any one of Embodiments 134-148, further comprising a neutralizing antibody stain buffer, a neutralizing antibody stain, 1% BSA/PBS, PBS, or any combinations thereof.

Embodiment 150. A composition comprising a mixture of at least one first type of identifiable microparticle conjugated to a SARS-CoV-2 S protein or fragment thereof.

Embodiment 151. The composition of Embodiment 150, further comprising an second type of identifiable microparticle conjugated to a SARS-CoV-2 nucleoprotein (NP) or a fragment thereof.

Embodiment 152. The composition of Embodiment 150 or 151, further comprising an additional type of identifiable microparticle conjugated to a full-length SARS-CoV-2 S protein.

Embodiment 153. The composition of any one of Embodiments 150-152, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 154. The composition of any one of Embodiments 150-153, wherein the SARS-CoV-2 S protein or a fragment thereof is subunit 1 (S1) or a fragment thereof.

Embodiment 155. The composition of any one of Embodiments 150-153, wherein the SARS-CoV-2 S protein or a fragment thereof is receptor binding domain (RBD) or a fragment thereof.

Embodiment 156. The composition of any one of Embodiments 150-155, further comprising a detectably labelled SARS-CoV-2 S protein receptor of fragment thereof.

Embodiment 157. The composition of Embodiment 156, wherein the SARS-CoV-2 S protein receptor or fragment thereof is human angiotensin-converting enzyme 2 (ACE-2) or a fragment thereof.

Embodiment 158. The composition of Embodiment 156 or 157, wherein the detectably labelled SARS-CoV-2 S protein receptor or fragment thereof is detectably labelled with a fluorescent molecule.

Embodiment 159. The composition of Embodiment 158, wherein the fluorescent molecule is phycoerythrin.

Embodiment 160. The composition of Embodiment 156 or 157, wherein the detectably labelled SARS-CoV-2 S protein receptor or fragment thereof is biotinylated and is detected with a streptavidin-labelled fluorescent molecule.

Embodiment 161. The composition of Embodiment 160, wherein the streptavidin-labelled fluorescent molecule is streptavidin-phycoerythrin.

Embodiment 162. The composition of any one of Embodiments 150-161, further comprising a SARS-CoV-2 neutralizing antibody.

Embodiment 163. The composition of any one of claims 150-162, further comprising a neutralizing antibody stain buffer, a neutralizing antibody stain, 1% BSA/PBS, PBS, or any combinations thereof.

Embodiment 164. A composition comprising a mixture of at least one identifiable microparticle conjugated to a SARS-CoV-2 S protein receptor or fragment thereof.

Embodiment 165. The composition of Embodiment 164, wherein the SARS-CoV-2 S protein receptor or fragment thereof is human angiotensin-converting enzyme 2 (ACE-2) or a fragment thereof.

Embodiment 166. The composition of Embodiment 164 or 165, wherein the microparticles are identifiable by size, magnetic properties, fluorescence, ultraviolet-excited fluorescence wavelength, violet-excited fluorescence wavelength, fluorescence intensity, metal isotopes, or any combination thereof.

Embodiment 167. The composition of Embodiments 164-166, further comprising a detectably labelled SARS-CoV-2 S protein of fragment thereof.

Embodiment 168. The composition of Embodiment 167, wherein the SARS-CoV-2 S protein or a fragment thereof is subunit 1 (S1) or a fragment thereof.

Embodiment 169. The composition of Embodiment 167, wherein the SARS-CoV-2 S protein or a fragment thereof is receptor binding domain (RBD) or a fragment thereof.

Embodiment 170. The composition of Embodiment 168 or 169, comprising two detectably labelled SARS-CoV-2 S proteins, RBDs, or fragments thereof from two different SARS-CoV-2 variants.

Embodiment 171. The composition of any one of Embodiments 164-170, wherein the detectably labelled SARS-CoV-2 S protein receptor or fragment thereof is detectably labelled with a fluorescent molecule.

Embodiment 172. The composition of Embodiment 170, wherein the fluorescent molecule is phycoerythrin.

Embodiment 173. The composition of any one of Embodiments 164-170, wherein the detectably labelled SARS-CoV-2 S protein receptor or fragment thereof is biotinylated and is detected with a streptavidin-labelled fluorescent molecule.

Embodiment 174. The composition of Embodiment 173, wherein the streptavidin-labelled fluorescent molecule is streptavidin-phycoerythrin.

Embodiment 175. The composition of any one of Embodiments 164-174, further comprising a SARS-CoV-2 neutralizing antibody.

Embodiment 176. The composition of any one of Embodiments 164-175, further comprising a neutralizing antibody stain buffer, a neutralizing antibody stain, 1% BSA/PBS, PBS, or any combinations thereof.

### EXAMPLES

Examples 1-14 and 16 were conducted using wild-type SARS-CoV-2 proteins and wild-type human ACE-2 (NCBI Gene ID: 59272).

### Example 1

### Assay for Detection of SARS-CoV-2 Neutralizing Antibodies Using RBD- or S1-Conjugated Microparticles

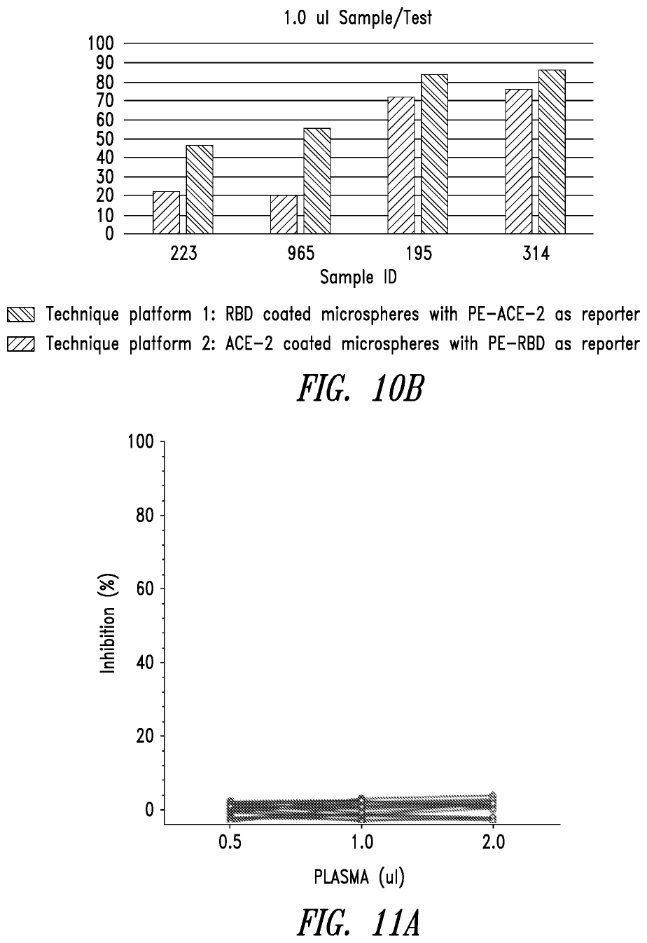

Test samples were assayed for the presence of SARS-CoV-2 neutralizing antibodies using Platform 1 (RBD-conjugated microparticles and labelled ACE-2) and Platform 2 (ACE-2-conjugated microparticles and labelled RBD). Four plasma samples with different levels of SARS-CoV-2 antibodies were tested in parallel. Five microliters of microspheres coated with RBD or ACE-2 were incubated with 50 μl 1% BSA diluted sample containing 0.5 μl (FIG. 10A) or 1.0 μl (FIG. 10B) of plasma for 30 minutes at room temperature in a well of a 96-well plate. The plate was washed three times with 150 μl 1% BSA/PBS and the microspheres were resuspended in 100 μl phycoerythrin (PE)-labeled ACE-2 (PE-ACE-2) or PE-labeled RBD (PE-RBD). After an additional 30-minute incubation at room temperature, the microspheres were washed and acquired in a Lyrics flow cytometer. The inhibition % was calculated as follows: inhibition %=(1−MFI of sample/MFI of PBS)×100%. Platform 1 was found to be more sensitive for detection of SARS-CoV-2 neutralizing antibodies than Platform 2. Data are shown in Table 1 and FIG. 10A and FIG. 10B.

TABLE 1

| | Inhibition (%) | | | |
|---|---|---|---|---|
| | Sample 0.5 μl | | Sample 1.0 μl | |
| | Platform 2 | Platform 1 | Platform 2 | Platform 1 |
| Sample ID | PE-RBD) | (PE-ACE-2) | (PE-RBD) | PE-ACE-2) |
| 223 | 15 | 27 | 22 | 46 |
| 965 | 15 | 39 | 20 | 55 |
| 195 | 48 | 72 | 72 | 84 |
| 314 | 60 | 74 | 76 | 86 |

### Example 2

### Assay for Detection of SARS-CoV-2 Neutralizing Antibodies Using RBD-Conjugated Microparticles

Test samples were assayed for the presence of SARS-CoV-2 neutralizing antibodies using Platform 1 (RBD-conjugated microparticles and labelled ACE-2). A total of 20 SARS-CoV-2 antibody negative plasma samples (FIG. 11A) from patients never exposed to SARS-CoV-2 and 30 SARS-CoV-2 antibody positive plasma samples (FIG. 11B) as confirmed by RT-PCR were tested. Five microliters (μl) of microspheres coated with RBD were incubated with 50 μl 1% BSA diluted sample containing 0.5 μl, 1.0 μl or 2 μl of plasma for 30 minutes at room temperature in a well of a 96-well plate. The plate was washed three times with 150 μl 1% BSA/PBS and the microspheres were resuspended in 100 μL PE-labeled ACE-2 (PE-ACE-2). After an additional 30-minute incubation at room temperature, the microspheres were washed and acquired in a Lyrics flow cytometer. The inhibition % was calculated as follows: inhibition %=(1−MFI of sample/MFI of PBS)×100%. Dose dependent inhibitions were observed in SARS-CoV-2 antibody positive samples, and no inhibition was observed in SARS-CoV-2 antibody negative samples.

### Example 3

### Detection of Dose-Dependent Inhibition of RBD/Ace-2 Binding by SARS-CoV-2 Antibodies Using RBD-Conjugated Microparticles

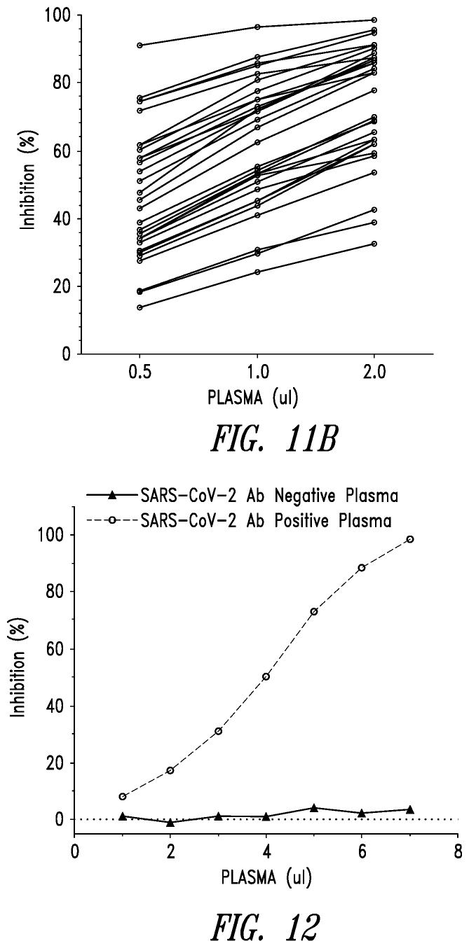

Serial dilutions of one SARS-CoV-2 antibody negative plasma sample and one SARS-CoV-2 antibody positive plasma sample were assayed using Platform 1 (RBD-conjugated microparticles and labelled ACE-2). Five microliters of microspheres coated with RBD were incubated with serial dilutions of one SARS-CoV-2 antibody negative plasma sample and one SARS-CoV-2 antibody positive plasma sample for 30 minutes at room temperature in a well of a 96-well plate. The plate was washed three times with 150 μl 1% BSA/PBS and the microspheres were resuspended in 100 μl PE-labeled ACE-2 (PE-ACE-2). After an additional 30-minute incubation at room temperature, the microspheres were washed and acquired in a Lyrics flow cytometer. The inhibition % was calculated as follows: inhibition %=(1−MFI of sample/MFI of PBS)×100%. A dose-dependent inhibition was observed in the SARS-CoV-2 antibody positive plasma sample, but not in the SARS-CoV-2 antibody negative plasma sample. Data are shown in FIG. 12.

### Example 4

### Detection of Dose-Dependent Inhibition of RBD/Ace-2 Binding by Free RBD Using RBD-Conjugated Microparticles

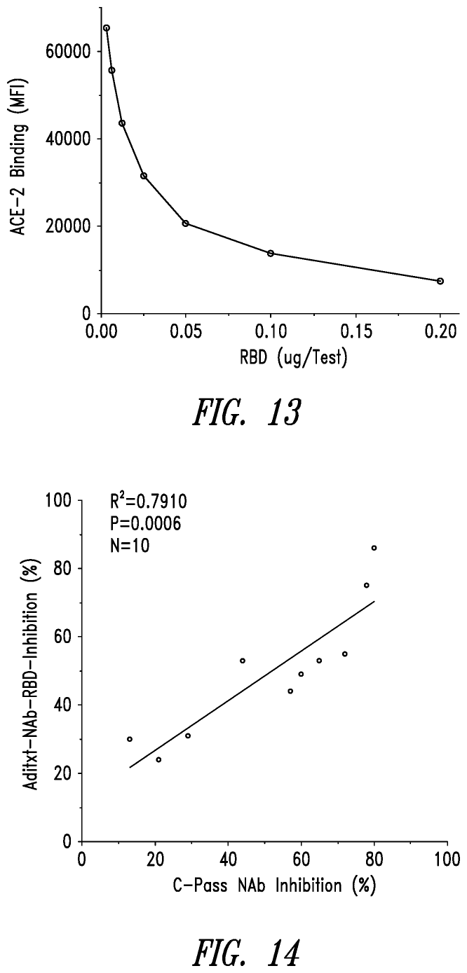

The ability of free RBD to inhibit RBD/ACE-2 binding was assayed. Five microliters of microspheres coated with RBD were incubated with 50 μl PE-ACE-2 in the presence of various concentrations of free pure RBD protein for 30 minutes at room temperature in a well of a 96-well plate. The plate was washed three times with 150 μl 1% BSA/PBS and the microspheres were acquired in a Lyrics flow cytometer. The inhibition % was calculated as follows: inhibition %=(1−MFI of sample/MFI of PBS)×100%. A dose-dependent inhibition of the binding of PE-ACE-2 to the solid phase RBD on the microspheres was observed. Data are shown in FIG. 13.

### Example 5

### Comparison of Assay Using RBD-Conjugated Microparticles and ELISA-Based Assay for Detection of SARS-CoV-2 Neutralizing Antibodies

Test samples were assayed for the presence of SARS-CoV-2 neutralizing antibodies using Platform 1 (RBD-conjugated microparticles and labelled ACE-2) and using the cPass™ ELISA-based assay (Genscript). Ten SARS-CoV-2 antibody positive plasma samples were tested in parallel. A simplified, one-step version of the Platform 1 assay was used. Five microliters of microspheres coated with RBD were incubated with 50 μl 1% BSA diluted sample containing 0.5 μl, 1.0 μl or 2 μl of plasma for 30 minutes at room temperature in a well of a 96-well plate. The plate was washed three times with 150 μl 1% BSA/PBS and the microspheres were resuspended in 100 μL PE-labeled ACE-2 (PE-ACE-2). After an additional 30-minute incubation at room temperature, the microspheres were washed and acquired in a Lyrics flow cytometer. The inhibition % was calculated as follows: inhibition %=(1−MFI of sample/MFI of PBS)×100%. The cPass™ assay was carried out according to the manufacturer's instructions. Briefly, diluted samples and controls were mixed 1:1 with HRP-labeled RBD and the mixtures were incubated at 37ºC for 30 minutes. 100 μl of each mixture was then added to individual wells of an ACE-2-coated 96-well plate and incubated at 37ºC for 15 minutes. After four washes, 100 μl of TMB was added to each well and the plate was incubated in the dark at room temperature for 15 minutes. 50 μl of stop solution was added to each well, and the 450 nm absorbance of each well was measured. The results showed 100% concordance in all SARS-CoV-2 antibody positive samples (N=30) between the two methods, but only 65% concordance in SARS-CoV-2 antibody negative samples (N=13). Seven SARS-CoV-2 antibody negative samples tested positive by cPass™, but negative by the one-step Platform 1 assay. This suggests that the cPass™ ELISA-based assay has provided some false positive results. Data are shown in Table 2 and FIG. 14. In Table 2, the cutoff for negative/positive for Nab-Platform 1 was 10% and the cutoff for negative/positive for GenScript C-Pass was 20%.

TABLE 2

| SARS- | | Nab - Platform 1 | | Genscript C-Pass | |
|---|---|---|---|---|---|
| CoV-2 Ab | Sample ID | Nab % | Result | Nab % | Result |
| Negative | 186 | −2 | Negative | 1 | Negative |
| | 190 | −3 | Negative | 3 | Negative |
| | 198 | 2 | Negative | 3 | Negative |
| | 200 | −1 | Negative | 4 | Negative |
| | 203 | 1 | Negative | 4 | Negative |
| | 207 | 0 | Negative | 2 | Negative |
| | 299 | −1 | Negative | 6 | Negative |
| | 362 | −3 | Negative | 12 | Negative |
| | 404 | −1 | Negative | 16 | Negative |
| | 405 | 3 | Negative | 24 | Positive |
| | 416 | 3 | Negative | 21 | Positive |
| | 419 | −2 | Negative | 22 | Positive |
| | 437 | 2 | Negative | 21 | Positive |
| | 440 | −1 | Negative | 21 | Positive |
| | 441 | 1 | Negative | 22 | Positive |
| | 448 | 1 | Negative | 21 | Positive |
| | 420 | 0 | Negative | 19 | Negative |
| | 426 | 1 | Negative | 17 | Negative |
| | 433 | 2 | Negative | 12 | Negative |
| | 435 | 3 | Negative | 19 | Negative |
| Positive | 136 | 24 | Positive | 22 | Positive |
| | 194 | 85 | Positive | 60 | Positive |
| | 195 | 41 | Positive | 80 | Positive |
| | 197 | 53 | Positive | 79 | Positive |
| | 202 | 49 | Positive | 72 | Positive |
| | 206 | 86 | Positive | 44 | Positive |
| Negative | 186 | −2 | Negative | 1 | Negative |
| | 190 | −3 | Negative | 3 | Negative |
| | 198 | 2 | Negative | 3 | Negative |
| | 200 | −1 | Negative | 4 | Negative |
| | 203 | 1 | Negative | 4 | Negative |
| | 207 | 0 | Negative | 2 | Negative |
| | 299 | −1 | Negative | 6 | Negative |
| | 362 | −3 | Negative | 12 | Negative |
| | 404 | −1 | Negative | 16 | Negative |
| | 405 | 3 | Negative | 24 | Positive |
| | 416 | 3 | Negative | 21 | Positive |
| | 419 | −2 | Negative | 22 | Positive |
| | 437 | 2 | Negative | 21 | Positive |
| | 440 | −1 | Negative | 21 | Positive |
| | 441 | 1 | Negative | 22 | Positive |
| | 448 | 1 | Negative | 21 | Positive |
| | 420 | 0 | Negative | 19 | Negative |
| | 426 | 1 | Negative | 17 | Negative |
| | 433 | 2 | Negative | 12 | Negative |
| | 435 | 3 | Negative | 19 | Negative |
| | 209 | 75 | Positive | 65 | Positive |
| | 214 | 51 | Positive | 29 | Positive |
| | 221 | 72 | Positive | 13 | Positive |
| | 223 | 55 | Positive | 57 | Positive |
| | 156 | 53 | Positive | 84 | Positive |
| | 171 | 45 | Positive | 47 | Positive |
| | 173 | 53 | Positive | 69 | Positive |
| | 199 | 75 | Positive | 65 | Positive |
| | 201 | 45 | Positive | 84 | Positive |
| | 208 | 31 | Positive | 70 | Positive |
| | 211 | 81 | Positive | 80 | Positive |
| | 213 | 72 | Positive | 77 | Positive |
| | 216 | 72 | Positive | 83 | Positive |
| | 217 | 30 | Positive | 81 | Positive |
| | 220 | 44 | Positive | 85 | Positive |
| | 224 | 78 | Positive | 85 | Positive |
| | 227 | 69 | Positive | 86 | Positive |
| | 234 | 73 | Positive | 85 | Positive |
| | 236 | 62 | Positive | 86 | Positive |
| | 242 | 96 | Positive | 86 | Positive |
| | 012236 | 83 | Positive | 87 | Positive |
| | 012410 | 88 | Positive | 96 | Positive |
| | 112778 | 67 | Positive | 83 | Positive |
| | 213178 | 54 | Positive | 69 | Positive |

### Example 6

### Two-Microparticle Assay for Detection of SARS-CoV-2 Neutralizing Antibodies

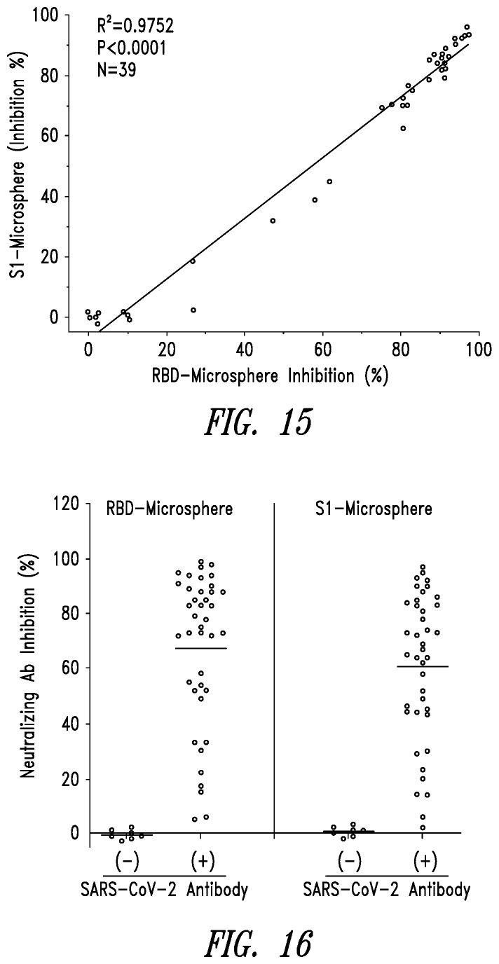

Test samples were assayed for the presence of SARS-CoV-2 neutralizing antibodies using Platform 3, in which the assay uses two types of identifiably labelled microparticles. The three-microparticle version of Platform 3 was used, which employs identifiably labelled microparticles conjugated with SARS-CoV-2 RBD and S1. Each of the species of microsphere had different fluorescence properties. A total of 39 plasma samples were tested. Five microliters of a mix of three species of microspheres—one species coated with RBD and one species coated with S1—were incubated with 50 μl 1% BSA diluted sample containing 1.0 μl plasma for 30 minutes at room temperature in a well of a 96-well plate. The plate was washed three times with 150 μl 1% BSA/PBS and the microspheres were resuspended in 100 μl PE-labeled ACE-2 (PE-ACE-2). After an additional 30-minute incubation at room temperature, the microspheres were washed and acquired in a Lyrics flow cytometer. The RBD and S1 microsphere populations were gated, and the PE fluorescence intensity was measured. The inhibition % was calculated as follows: inhibition %=(1−MFI of sample/MFI of PBS)×100%. The neutralization inhibition rates between RBD- and S1-microspheres demonstrated good correlation (R2=0.9752; P<0.0001), except for four samples that showed different inhibition rates between RBD and S1 microspheres (indicated with * in Table 3). Data are shown in FIG. 15 and Table 3.

TABLE 3

% Inhibition

| Sample | RBD-microspheres | S1-microspheres |
|---|---|---|
| 1 | 2.45 | −2.22 |
| 2* | 10.53 | −0.6 |
| 3 | 0.33 | −0.06 |
| 4 | 1.85 | 0.06 |
| 5* | 10.04 | 0.85 |
| 6 | 2.55 | 1.32 |
| 7* | 8.94 | 1.9 |
| 8 | −0.24 | 1.96 |
| 9* | 26.92 | 2.55 |
| 10 | 26.66 | 18.58 |
| 11 | 47.25 | 32.13 |
| 12 | 58.03 | 39.01 |
| 13 | 61.78 | 45.01 |
| 14 | 80.72 | 62.61 |
| 15 | 75.27 | 69.39 |
| 16 | 80.54 | 70.2 |
| 17 | 81.65 | 70.23 |
| 18 | 77.83 | 70.49 |
| 19 | 80.66 | 72.76 |
| 20 | 83.02 | 75.21 |
| 21 | 81.96 | 76.69 |
| 22 | 87.32 | 78.72 |
| 23 | 91.28 | 79.3 |
| 24 | 90.61 | 81.89 |
| 25 | 91.39 | 82.44 |
| 26 | 91.3 | 84.12 |
| 27 | 89.43 | 84.23 |
| 28 | 87.45 | 85.21 |
| 29 | 90.44 | 86.12 |
| 30 | 92.38 | 86.43 |
| 31 | 90.61 | 87.12 |
| 32 | 88.65 | 87.19 |
| 33 | 91.49 | 89.13 |
| 34 | 94.18 | 90.63 |
| 35 | 93.91 | 92.32 |
| 36 | 95.72 | 92.44 |
| 37 | 96.45 | 93.33 |
| 38 | 97.47 | 93.46 |
| 39 | 97.09 | 96.09 |

### Example 7

### Comparison of Three-Microparticle Assay and Cell-Based Assay for Detection of SARS-CoV-2 Neutralizing Antibodies

Test samples were assayed for the presence of SARS-CoV-2 neutralizing antibodies using Platform 3 as described in Example 6 and using the IMMUNO-COV™ cell-based bioassay (Imanis Life Sciences). Forty samples were tested in parallel, including 20 plasma samples and 20 serum samples. The IMMUNO-COV™ assay was carried out according to the manufacturer's instructions. Briefly, Vero-ACE-2 cells were seeded at 1×10⁴ cells/well in 96-well plates 16 to 24 hours before being used for assays. On the day of assay, test samples and controls were prepared and mixed with VSV-SARS2-Fluc, a VSV pseudotyped with SARS-CoV-2 S protein and carrying a luciferase marker gene, in U-bottom suspension cell culture plates to a final volume of 240 μl per well. Virus was used at 300 pfu/well. Virus mixtures in U-well plates were incubated at room temperature for 30-45 minutes, then 100 μl of each mix was overlaid onto the Vero-ACE-2 monolayer in duplicate. Plates were incubated at 37° ° C. and 5% CO₂ for 24 to 28 hours. D-luciferin was added to wells and luminescence was measured. The concordance rate between the two assays was 100%. Data are shown in Table 4. LOD is Limit of Detection; VNT² is Virus Neutralizing Titer.

TABLE 4

| | | Platform 3 (multiplex) inhibition % | | | | |
|---|---|---|---|---|---|---|
| | | RBD- | S1- | | IMMUNO- | |
| | | micro- | micro- | | COV ™ | |
| Sample | | particles | particles | Results | VNT² | Result |
| Plasma | 1 | 75 | 70 | Pos | 513 | Pos |
| | 2 | 94 | 93 | Pos | >2400 | Pos |
| | 3 | 70 | 64 | Pos | 325 | Pos |
| | 4 | 36 | 30 | Pos | 553 | Pos |
| | 5 | 22 | 14 | Pos | <LOD | Neg |
| | 6 | 26 | 24 | Pos | 225 | Pos |
| | 7 | 97 | 97 | Pos | >2400 | Pos |
| | 8 | 1 | −1 | Neg | <LOD | Neg |
| | 9 | 15 | 9 | Pos | 41 | Pos |
| | 10 | 70 | 68 | Pos | >2400 | Pos |
| | 11 | 1 | 0 | Neg | <LOD | Neg |
| | 12 | 1 | 0 | Neg | <LOD | Neg |
| | 13 | 0 | 0 | Neg | <LOD | Neg |
| | 14 | 0 | 0 | Neg | <LOD | Neg |
| | 15 | 1 | 0 | Neg | <LOD | Neg |
| | 16 | 1 | 0 | Neg | <LOD | Neg |
| | 17 | 22 | 4 | Pos | 44 | Pos |
| | 18 | 42 | 40 | Pos | 1246 | Pos |
| | 19 | 70 | 63 | Pos | 1969 | Pos |
| | 20 | 100 | 99 | Pos | >3200 | Pos |
| Serum | 21 | 0 | 1 | Neg | <LOD | Neg |
| | 22 | 2 | 3 | Neg | <LOD | Neg |
| | 23 | 0 | 0 | Neg | <LOD | Neg |
| | 24 | 0 | 0 | Neg | <LOD | Neg |
| | 25 | 1 | 1 | Neg | <LOD | Neg |
| | 26 | 22 | 11 | Pos | 72 | Pos |
| | 27 | 0 | 0 | Neg | <LOD | Neg |
| | 28 | 97 | 95 | Pos | >2400 | Pos |
| | 29 | 78 | 65 | Pos | 1200 | Pos |
| | 30 | 99 | 97 | Pos | >2400 | Pos |
| | 31 | 73 | 73 | Pos | 1202 | Pos |
| | 32 | 90 | 86 | Pos | >2400 | Pos |
| | 33 | 83 | 78 | Pos | 1094 | Pos |
| | 34 | 94 | 90 | Pos | >2400 | Pos |
| | 35 | 22 | 20 | Pos | 634 | Pos |
| | 36 | 49 | 43 | Pos | 921 | Pos |
| | 37 | 88 | 83 | Pos | >2400 | Pos |
| | 38 | 30 | 23 | Pos | 274 | Pos |
| | 39 | 33 | 29 | Pos | 268 | Pos |
| | 40 | 58 | 49 | Pos | 563 | Pos |

### Example 8

### Three Microparticle Assay for Detection of SARS-CoV-2 Neutralizing Antibodies Using Serum Samples

Serum samples were assayed for the presence of SARS-CoV-2 neutralizing antibodies using Platform 3, carried out as in Example 6, but with three microparticles. Seven SARS-CoV-2 antibody negative serum samples and 40 SARS-CoV-2 antibody positive serum samples were tested. A significant difference in the rate of inhibition of ACE-2 binding was observed between the SARS-CoV-2 antibody negative serum samples and the SARS-CoV-2 antibody positive serum samples using both RBD-conjugated microparticles and S1-conjugated microparticles (P<0.0001). Data are shown in FIG. 16.

### Example 9

### Three-Particle Assay for Detection of SARS-CoV-2 Neutralizing Antibodies Using Pre- and Post-Vaccination Finger-Stick Samples

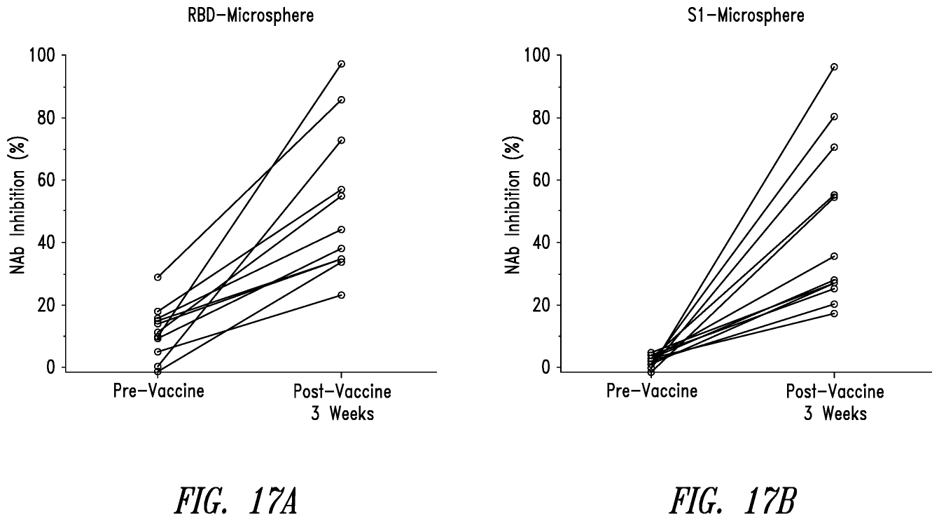

Plasma samples derived from blood obtained by finger-stick were assayed for the presence of SARS-CoV-2 neutralizing antibodies using Platform 3 as set forth in Example 6, but with three microparticles. Samples were collected from 11 individuals before vaccination and three weeks post-SARS-CoV-2 vaccination. Increased levels of neutralizing antibodies were observed in all individuals after vaccination using both RBD-conjugated microparticles (FIG. 17A) and S1-conjugated microparticles (FIG. 17B) (P<0.0001). Data are shown in Table 5 and FIG. 17A and FIG. 17B.

TABLE 5

| | Inhibition % | | | | |
|---|---|---|---|---|---|
| | RBD-conjugated | | S1-conjugated | | |
| | microparticles | | microparticles | | |
| Sample | Pre- | Post- | Pre- | Post- | Vaccine |
| ID | vaccine | vaccine | vaccine | vaccine | vendor |
| 5 | 16 | 44 | 0 | 34 | Pfizer |
| 38 | 10 | 97 | −3 | 96 | Pfizer |
| 42 | 18 | 57 | 0 | 54 | Pfizer |
| 1 | 15 | 35 | 2 | 23 | Moderna |
| 21 | 5 | 23 | −1 | 18 | Moderna |
| 23 | 11 | 55 | −5 | 53 | Moderna |
| 24 | 29 | 86 | −2 | 80 | Moderna |
| 28 | −1 | 34 | −2 | 25 | Moderna |
| 30 | 0 | 73 | −3 | 70 | Moderna |
| 44 | 9 | 38 | 0 | 15 | Moderna |
| 80 | 14 | 35 | 0 | 26 | Moderna |

### Example 10

### One-Step Assay for Detection of SARS-CoV-2 Neutralizing Antibodies

Test samples were assayed for the presence of SARS-CoV-2 neutralizing antibodies using a simplified, one-step version of the Platform 1 assay (RBD-conjugated microparticles and labelled ACE-2). 23 plasma samples derived from blood obtained by finger-stick were tested. Five microliters of a mixture of RBD-conjugated microspheres, S1-conjugated microspheres, and NP-conjugated microspheres were incubated with 20 μl diluted plasma (containing 1 μl of plasma), 20 μl of biotinylated ACE-2 (Bio-ACE-2), and 5 μl of streptavidin-phycoerythrin (SA-PE) for 60 minutes at RT. After washing twice with 150 μl PBS, the microspheres were resuspended in 60 μl PBS and were acquired in a Lyrics flow cytometer.

The same samples were assayed using a two-step version of the Platform 1 assay. Five microliters of a mixture of RBD-conjugated microspheres, S1-conjugated microspheres, and NP-conjugated microspheres were incubated with 20 μl diluted plasma and 20 μl of Bio-ACE-2 for 30 minutes at RT. After three washes with 150 μl PBS, the microspheres were resuspended in 50 μl of SA-PE and the plate was incubated for an additional 30 minutes. The plate was washed again and the microspheres were acquired in a Lyrics flow cytometer.

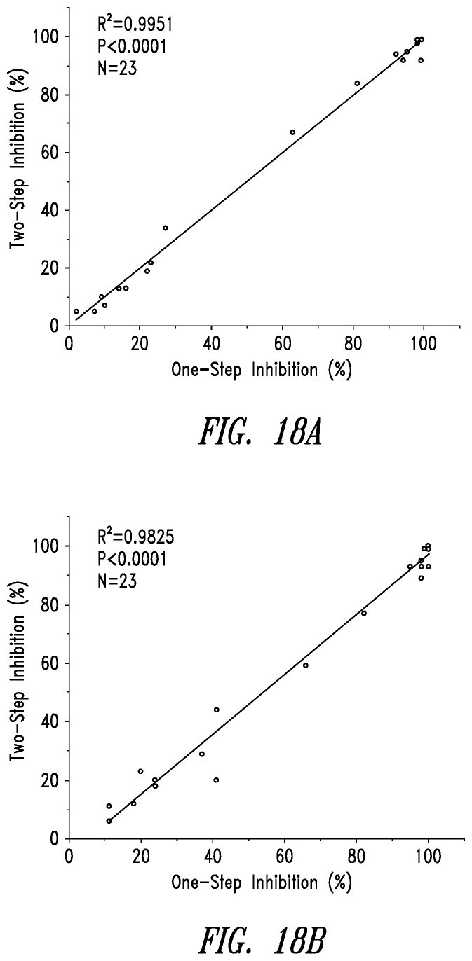

For both assays, the inhibition % was calculated as follows: inhibition %=(1-MFI of sample/MFI of PBS)×100%. Surprisingly, good correlation was observed between the one-step and two-step procedures despite the addition of sample, Bio-ACE-2 and SA-PE to the microspheres in a single step in the one-step procedure. Results are shown in FIG. 18A (S1-conjugated microspheres) and FIG. 18B (RBD-conjugated microspheres).

### Example 11

### Detection of SARS-CoV-2 Neutralizing Antibodies Using S-Conjugated Microparticles

Test samples were assayed for the presence of SARS-CoV-2 using microparticles conjugated to full-length S protein. Five microliters of S-conjugated microspheres were incubated with 20 μl diluted plasma (containing 1 μl of plasma), 20 μl of biotinylated ACE-2 (Bio-ACE-2), and 5 μl of streptavidin-phycoerythrin (SA-PE) for 60 minutes at RT. After washing twice with 150 μl PBS, the microspheres were resuspended in 60 μl PBS and were acquired in a Lyrics flow cytometer. The inhibition % was calculated as follows: inhibition %=(1−MFI of sample/MFI of PBS)×100%.

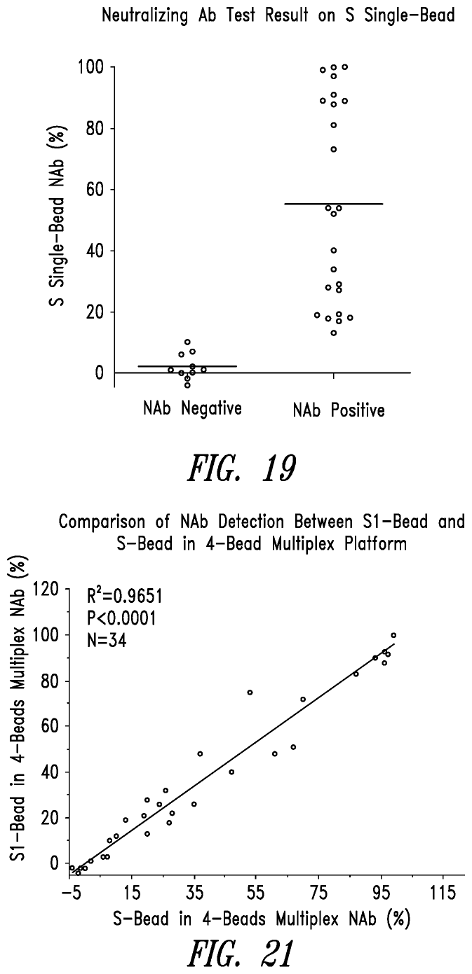

Ten known NAb negative and 18 NAb positive samples were tested. Results are shown in FIG. 19. The result showed 100% concordance with three-microparticle multiplex array method (i.e., identifiably labelled RBD-conjugated microspheres, S1-conjugated microspheres, and NP-conjugated microspheres).

### Example 12

### Four-Microparticle Multiplex Assay for Detection of SARS-CoV-2 Neutralizing Antibodies

Test samples were assayed for the presence of SARS-CoV-2 neutralizing antibodies using Platform 3, which is a multiplex assay that uses identifiably labelled microparticles. In one set of tests, a three-microparticle version of Platform 3 was used, which employs identifiably labelled microparticles conjugated with SARS-CoV-2 RBD, S1, or NP, as described in Example 10. Briefly, five microliters of a mixture of RBD-conjugated microspheres, S1-conjugated microspheres, and NP-conjugated microspheres were incubated with 20 μl diluted plasma (containing 1 μl of plasma), 20 μl of biotinylated ACE-2 (Bio-ACE-2), and 5 μl of streptavidin-phycoerythrin (SA-PE) for 60 minutes at RT. After washing twice with 150 μl PBS, the microspheres were resuspended in 60 μl PBS and were acquired in a Lyrics flow cytometer.

A four-microparticle version of Platform 3 was also used, which employs identifiably labelled microparticles conjugated with SARS-CoV-2 full-length S protein, RBD, S1, or NP. Each of the species of microsphere had different fluorescence properties. A total of 34 samples were tested. Five microliters of a mix of four species of microspheres—one species coated with RBD, one species coated with S1, one species coated with S, and one species coated with NP protein—were incubated with 20 μl diluted plasma (containing 1 μl of plasma), 20 μl of biotinylated ACE-2 (Bio-ACE-2), and 5 μl of streptavidin-phycoerythrin (SA-PE) for 60 minutes at RT. After washing twice with 150 μl PBS, the microspheres were resuspended in 60 μl PBS and were acquired in a Lyrics flow cytometer.

For both assays, the inhibition % was calculated as follows: inhibition %=(1-MFI of sample/MFI of PBS)×100%.

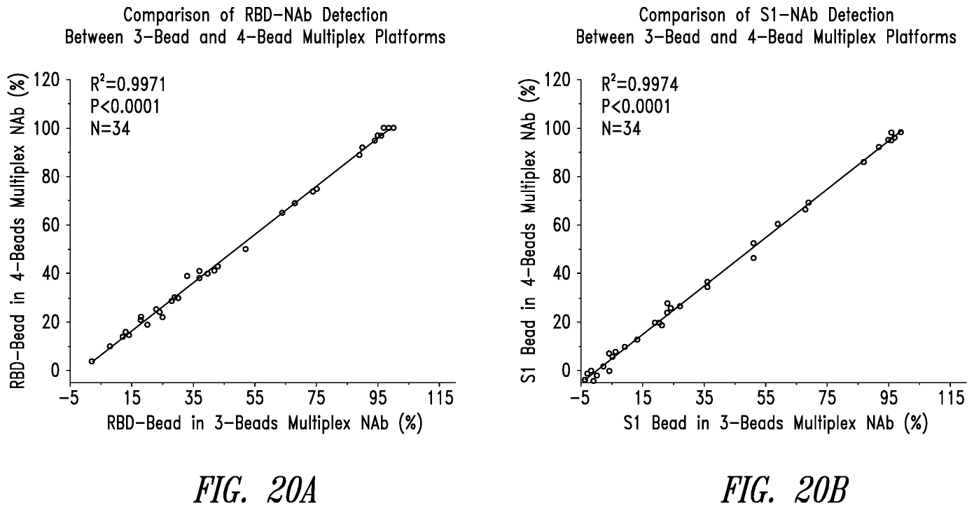

The neutralization inhibition rates between RBD microspheres (FIG. 20A) and S1microspheres (FIG. 20B) demonstrated good correlation in both the three-microparticle and four-microparticle assays. In the for-microparticle assay, good correlation between full-length S- and S1-microspheres was observed (FIG. 21). Results are shown in FIG. 20 and FIG. 21 and in Table 6.

TABLE 6

Neutralizing Antibody (%)

| | | |
|---|---|---|
| RBD | S1 | S |

| Sample | 3-Bead | 4-Bead | 3-Bead | 4-Bead | 4-Bead |
|---|---|---|---|---|---|
| 1 | 14 | 15 | 4 | 7 | 3 |
| 2 | 13 | 16 | 9 | 10 | 12 |
| 3 | 96 | 97 | 97 | 97 | 92 |
| 4 | 52 | 50 | 51 | 47 | 40 |
| 5 | 95 | 97 | 95 | 96 | 93 |
| 6 | 18 | 22 | −2 | 0 | −2 |
| 7 | 90 | 92 | 92 | 93 | 90 |
| 8 | 37 | 41 | 23 | 24 | 26 |
| 9 | 29 | 30 | 20 | 20 | 28 |
| 10 | 24 | 24 | 2 | 2 | 1 |
| 11 | 43 | 43 | 27 | 27 | 18 |
| 12 | 14 | 15 | −1 | −4 | −3 |
| 13 | 64 | 65 | 59 | 61 | 48 |
| 14 | 2 | 4 | −4 | −4 | −2 |
| 15 | 25 | 22 | 4 | 0 | −2 |
| 16 | 40 | 40 | 36 | 35 | 26 |
| 17 | 12 | 14 | 0 | −2 | −4 |
| 18 | 8 | 10 | 5 | 6 | 3 |
| 19 | 100 | 100 | 99 | 99 | 100 |
| 20 | 99 | 100 | 99 | 99 | 100 |
| 21 | 97 | 100 | 96 | 99 | 100 |
| 22 | 33 | 39 | 23 | 28 | 22 |
| 23 | 37 | 38 | 13 | 13 | 19 |
| 24 | 74 | 74 | 68 | 67 | 51 |
| 25 | 28 | 29 | 21 | 19 | 21 |
| 26 | 20 | 19 | 19 | 20 | 13 |
| 27 | 94 | 95 | 96 | 96 | 88 |
| 28 | 23 | 25 | 6 | 8 | 10 |
| 29 | 18 | 21 | −3 | −1 | −2 |
| 30 | 89 | 89 | 87 | 87 | 83 |
| 31 | 68 | 69 | 51 | 53 | 75 |
| 32 | 75 | 75 | 69 | 70 | 72 |
| 33 | 42 | 41 | 36 | 37 | 48 |
| 34 | 30 | 30 | 24 | 26 | 32 |

### Example 13

### Three-Microparticle Multiplex Assay for Detection of SARS-CoV-2 Neutralizing Antibodies

Further assays were conducted as set forth in Example 6 with either a single microsphere or three microspheres. Confirmation of SARS-CoV-2 infection via RT-PCR, symptoms at time of sample collection, if relevant, and vaccination dates are were also recorded. Results are provided in Tables 7 and 8.

TABLE 7
SARS-COV-2 Ab Random Sample Test Result - Vaccinated Patients

| | | | | | NAb % | | |
|---|---|---|---|---|---|---|---|
| | | | | | Single | Triple- | |
| Sample | RT- | | Vaccine | | Bead | Bead | |
| ID | PCR | Symptom | (Date) | Sample Date | RBD | RBD | S1 |
| 1 | 0 | − | V1-M: | Dec. 29, 2020 | 26 | | |
| | | | Feb. 1, 2021 | Jan. 12, 2021 | 32 | | |
| | | | V2, | Jan. 26, 2021 | | 15 | 2 |
| | | | Mar. 8, 2021 | Feb. 8, 2021 | | 16 | −4 |
| | | | | Feb. 13, 2021 | | 15 | 7 |
| | | | | Feb. 21, 2021 | | 35 | 23 |
| | | | | Mar. 1, 2021 | | 28 | 21 |
| | | | | Mar. 8, 2021 | | 33 | 24 |
| | | | | Mar. 15, 2021 | | 75 | 76 |
| | | | | Mar. 21, 2021 | | 78 | 80 |
| | | | | Apr. 11, 2021 | | 71 | 75 |
| 3 | − | − | V1-P: | Dec. 29, 2020 | 27 | | |
| | | | Feb. 24, 2021 | Feb. 24, 2021 | | 12 | 0 |
| | | | V2: | Mar. 9, 2021 | | 14 | 0 |
| | | | Mar. 17, 2021 | Mar. 27, 2021 | | 80 | 78 |
| | | | | Apr. 9, 2021 | | 73 | 71 |
| | | | | Apr. 17, 2021 | | 77 | 74 |
| | | | | May 9, 2021 | | 60 | 56 |
| | | | | Jul. 2, 2021 | | 62 | 58 |
| 4 | − | − | V1(P), | Dec. 29, 2020 | 28 | | |
| | | | Feb. 24, 2021 | Feb. 24, 2021 | | 25 | 4 |
| | | | V2: | Mar. 27, 2021 | | 95 | 95 |
| | | | Mar. 17, 2021 | May 9, 2021 | | 78 | 79 |
| 5 | − | − | V1-M: | Dec. 29, 2020 | 43 | 16 | 0 |
| | | | Jan. 4, 2021 | Jan. 21, 2021 | | 44 | 34 |
| | | | V2-M: | Feb. 7, 2021 | | 99 | 99 |
| | | | Feb. 1, 2021 | Apr. 9, 2021 | | 82 | 83 |
| | | | | May 9, 2021 | | 69 | 68 |
| 6 | − | − | V1-P: | Dec. 29, 2020 | 41 | 10 | 0 |
| | | | Dec. 22, 2020, | Jan. 12, 2021 | 64 | 45 | 40 |
| | | | V2-P: | Feb. 7, 2021 | | 84 | 82 |
| | | | Jan. 11, 2021 | Feb. 25, 2021 | | 70 | 65 |
| | | | | Mar. 9, 2021 | | 62 | 54 |
| | | | | Apr. 9, 2021 | | 53 | 42 |
| | | | | May 9, 2021 | | 34 | 24 |
| 7 | (−) | (+) | V1-P | Dec. 29, 2020 | 20 | | |
| | | | Feb. 24, 2021 | Jan. 12, 2021 | 22 | | |
| | | | V2: | Feb. 25, 2021 | | 14 | −1 |
| | | | Mar. 17, 2021 | Mar. 9, 2021 | | 28 | 16 |
| | | | | Apr. 9, 2021 | | 64 | 66 |
| 8 | (−) | | V1-P, | Dec. 29, 2020 | 9 | | |
| | | | Feb. 24, 2021 | Feb. 25, 2021 | | 2 | −4 |
| | | | V2: | Mar. 9, 2021 | | 27 | 20 |
| | | | Mar. 17, 2021 | Apr. 9, 2021 | | 77 | 78 |
| 9 | − | − | V1-P: | Dec. 29, 2020 | 19 | 7 | 0 |
| | | | Dec. 22, 2020 | Jan. 26, 2021 | | 91 | 87 |
| | | | V2, | | | | |
| | | | Jan. 11, 2021 | | | | |
| 11 | − | − | V1-M: | Dec. 29, 2020 | 12 | 7 | −2 |
| | | | Dec. 28, 2020 | Jan. 12, 2021 | | | |
| | | | V2, | Feb. 12, 2021 | 51 | | |
| | | | Jan. 28, 2021 | | | 99 | 99 |
| 12 | | None | V1(P): | Dec. 29, 2020 | 33 | | |
| | | | Mar. 22, 2021 | Mar. 22, 2021 | | 0 | −3 |
| | | | V2: | Apr. 11, 2021 | | 8 | 6 |
| | | | Apr. 13, 2021 | Apr. 29, 2021 | | 34 | 27 |
| 14 | − | − | V1-P: | Dec. 29, 2020 | 37 | 25 | 0 |
| | | | Jan. 23, 2021 | Feb. 1, 2021 | | 26 | −1 |
| | | | V2, | Feb. 12, 2021 | | 36 | 12 |
| | | | Feb. 13, 2021 | Feb. 19, 2021 | | 78 | 66 |
| | | | | Mar. 1, 2021 | | 68 | 63 |
| 15 | ? | ? | V1-M: | Dec. 29, 2020 | 12 | | |
| | | | Jan. 25, 2021 | Jan. 25, 2021 | | | |
| | | | V2, | Feb. 19, 2021 | | 30 | 25 |
| | | | Feb. 23, 2021 | | | | |
| 17 | (−) | None | V1(P): | ′ | 16 | | |
| | | | Jun. 9, 2021 | Jan. 11, 2021 | 6 | | |
| | | | V2: | Mar. 21, 2021 | | 19 | −1 |
| | | | Jun. 30, 2021 | Jul. 15, 2021 | | | |
| 19 | (−) | Cough/ | V1-P: | Jan. 24, 2021 | | 14 | 4 |
| | | Fatigue since | Jan. 23, 2021 | Feb. 9, 2021 | | 19 | 7 |
| | | vaccine | V2, | Feb. 21, 2021 | | 26 | 14 |
| | | | Feb. 13, 2021 | Feb. 28, 2021 | | 40 | 36 |
| | | | | Mar. 7, 2021 | | 91 | 93 |
| | | | | Mar. 15, 2021 | | 85 | 88 |
| 20 | − | − | V1-M: | Jan. 3, 2021 | 33 | 25 | 22 |
| | | | Dec. 23, 2020, | Jan. 19, 2021 | N/A | | |
| | | | V2-M: | Jan. 27, 2021 | | 90 | 92 |
| | | | Jan. 20, 2021 | Feb. 2, 2021 | | 94 | 95 |
| | | | | Feb. 10, 2021 | | 87 | 90 |
| | | | | Feb. 23, 2021 | | 79 | 83 |
| | | | | Mar. 3, 2021 | | 77 | 80 |
| | | | | Mar. 9, 2021 | | 74 | 76 |
| | | | | May 2, 2021 | | 56 | 55 |
| 21 | (−) | None | V1-M: | Jan. 3, 2021 | 10 | 5 | −1 |
| | | | Jan. 10, 2021, | Jan. 11, 2021 | 12 | 3 | −6 |
| | | | V2-M: | Jan. 17, 2021 | N/A | | |
| | | | Feb. 8, 2021 | Jan. 24, 2021 | | 11 | 10 |
| | | | | Jan. 31, 2021 | | 23 | 18 |
| | | | | Feb. 7, 2021 | | 21 | 18 |
| | | | | Feb. 21, 2021 | | 93 | 94 |
| | | | | Feb. 28, 2021 | | 89 | 91 |
| | | | | Mar. 7, 2021 | | 85 | 86 |
| | | | | May 2, 2021 | | 74 | 76 |
| 22 | (−) | (−) | | Nov. 27, 2020 | 21 | 27 | 15 |
| | | | | Dec. 21, 2020 | 23 | 28 | 18 |
| | (+) | (+) | | Dec. 31, 2020 | 100 | 100 | 99 |
| | | | | Feb. 9, 2021 | | 95 | 96 |
| | | | | Mar. 10, 2021 | | 86 | 87 |
| | | | | Apr. 7, 2021 | | 80 | 81 |
| | | | J&J | Apr. 21, 2021 | | 81 | 85 |
| | | | Apr. 8, 2021 | | | | |
| | | | | Jul. 21, 2021 | | 67 | 70 |
| 23 | 0 | None | V1-M: | Jan. 12, 2021 | 14 | 11 | −5 |
| | | | Jan. 13, 2021, | Jan. 19, 2021 | | | |
| | | | V2-M: | Jan. 27, 2021 | | 49 | 36 |
| | | | Feb. 11, 2021 | Feb. 3, 2021 | | 63 | 53 |
| | | | | Feb. 9, 2021 | | 55 | 53 |
| | | | | Feb. 22, 2021 | | 99 | 99 |
| | | | | Mar. 1, 2021 | | 98 | 98 |
| | | | | Mar. 8, 2021 | | 96 | 97 |
| | | | | May 3, 2021 | | 83 | 86 |
| 24 | 0 | None | V1-M: | Jan. 12, 2021 | 33 | 29 | −2 |
| | | | Jan. 12, 2021, | Jan. 18, 2021 | N/A | | |
| | | | V2-M: | Jan. 26, 2021 | | 76 | 70 |
| | | | Feb. 10, 2021 | Jan. 26, 2021 | | 74 | 69 |
| | | | | Feb. 3, 2021 | | 86 | 80 |
| | | | | Feb. 9, 2021 | | 80 | 78 |
| | | | | Feb. 22, 2021 | | 100 | 99 |
| | | | | Mar. 1, 2021 | | 100 | 99 |
| | | | | Mar. 8, 2021 | | 100 | 99 |
| | | | | May 3, 2021 | | 95 | 96 |
| 25 | 0 | None | V1(M): | Jan. 10, 2021 | 5 | | |
| | | | Feb. 19, 2021 | Feb. 18, 2021 | | 10 | −2 |
| | | | V2: | Feb. 26, 2021 | | 28 | −2 |
| | | | Mar. 17, 2021 | Mar. 4, 2021 | | 45 | 23 |
| | | | | Mar. 12, 2021 | | 44 | 24 |
| | | | | Mar. 18, 2021 | | 35 | 20 |
| | | | | Mar. 27, 2021 | | | |
| | | | | Apr. 2, 2021 | | | |
| | | | | Jun. 15, 2021 | | 70 | 63 |
| 28 | 0 | None | V1-M: | Jan. 12, 2021 | 0 | −1 | −2 |
| | | | Jan. 12, 2021, | Jan. 20, 2021 | | 6 | −5 |
| | | | V2-M: | Feb. 3, 2021 | | 42 | 27 |
| | | | Feb. 10, 2021 | Feb. 9, 2021 | | 34 | 25 |
| | | | | Feb. 17, 2021 | | 91 | 91 |
| | | | | Feb. 24, 2021 | | 85 | 86 |
| | | | | May 10, 2021 | | 59 | 55 |
| 30 | (−) | None | V1-M: | Jan. 9, 2021 | 0 | 0 | −3 |
| | | | Jan. 10, 2021, | Jan. 20, 2021 | | 9 | 5 |
| | | | V2-M: | Jan. 29, 2021 | | 63 | 60 |
| | | | Feb. 8, 2021 | Feb. 7, 2021 | | 73 | 70 |
| | | | | Feb. 16, 2021 | | 99 | 99 |
| | | | | Feb. 22, 2021 | | 98 | 97 |
| | | | | May 10, 2021 | | 84 | 83 |
| 32 | (−) | None | V1-M: | Jan. 12, 2021 | 42 | 29 | −5 |
| | | | Jan. 11, 2021, | Jan. 18, 2021 | | 29 | −2 |
| | | | V2-M: | Jan. 25, 2021 | | 50 | 34 |
| | | | Feb. 9, 2021 | Feb. 1, 2021 | | 58 | 37 |
| | | | | Feb. 8, 2021 | | 51 | 31 |
| | | | | Feb. 22, 2021 | | 94 | 95 |
| | | | | Mar. 1, 2021 | | 93 | 92 |
| | | | | Mar. 8, 2021 | | 89 | 87 |
| | | | | May 3, 2021 | | 73 | 73 |
| 34 | (−) | None | V1-M: | Jan. 12, 2021 | 38 | 32 | 23 |
| | | | Dec. 31, 2020 | | | | |
| 37 | 0 | None | V1-P: | Dec. 16, 2020 | 14 | 10 | −3 |
| | | | Dec. 31, 2020 | Jan. 13, 2021 | 23 | 21 | 6 |
| | | | V2, | Jan. 19, 2021 | 33 | 32 | 17 |
| | | | Jan. 21, 2021 | Jan. 28, 2021 | | 97 | 96 |
| | | | | Apr. 6, 2021 | | 84 | 81 |
| 39 | (−) | None | No | Jan. 11, 2021 | 14 | | |
| | | | | Jun. 4, 2021 | | | |
| | | | V1(M): | Jun. 18, 2021 | | 50 | 36 |
| | | | May 28, 2021 | Jun. 25, 2021 | | 50 | 35 |
| | | | V2: | Jul. 2, 2021 | | 94 | 95 |
| | | | Jun. 25, 2021 | | | | |
| 40 | (−) | None | V1(M): | Jan. 11, 2021 | 14 | | |
| | | | May 28, 2021 | Jan. 15, 2021 | 5//10 | −2 | 1 |
| | | | V2: | Apr. 1, 2021 | | 3 | −7 |
| | | | Jun. 25, 2021 | Jun. 4, 2021 | | | |
| | | | | Jun. 11, 2021 | | | |
| | | | | Jun. 18, 2021 | | 26 | 21 |
| | | | | Jun. 25, 2021 | | 34 | 27 |
| | | | | Jul. 2, 2021 | | 88 | 90 |
| 41 | 0 | None | V1-P: | Dec. 16, 2020 | 20 | 18 | 0 |
| | | | Jan. 7, 2021, | Jan. 12, 2021 | 14 | 12 | 0 |
| | | | V2-P: | Jan. 18, 2021 | 8 | 9 | 0 |
| | | | Jan. 27, 2021 | Jan. 27, 2021 | | 28 | 9 |
| | | | | Feb. 5, 2021 | | 57 | 54 |
| | | | | Feb. 10, 2021 | | 63 | 61 |
| | | | | Feb. 17, 2021 | | 64 | 60 |
| | | | | Mar. 31, 2021 | | 44 | 33 |
| 42 | 0 | None | V1(M): | Jan. 15, 2021 | 6/9 | 1 | −3 |
| | | | Apr. 14, 2021 | Apr. 1, 2021 | | 4 | −2 |
| | | | | Apr. 16, 2021 | | 9 | 3 |
| | | | | Apr. 22, 2021 | | 2 | −3 |
| | | | | Apr. 29, 2021 | | 18 | 12 |
| | | | V2: | May 6, 2021 | | 17 | 13 |
| | | | May 16, 2021 | | | | |
| | | | | May 21, 2021 | | 86 | 86 |
| 43 | (−) | None | V1-M: | Dec. 16, 2020 | 15 | 9 | 0 |
| | | | Jan. 18, 2021, | Jan. 15, 2021 | 23 | 20 | 0 |
| | | | V2-M: | (Jan. 25, 2021) | 27 | 18 | −2 |
| | | | Feb 15, 2021 | Feb. 1, 2021 | | 28 | 3 |
| | | | | Feb. 9, 2021 | | 38 | 15 |
| | | | | Feb. 15, 2021 | | 36 | 16 |
| | | | | | | 33 | 15 |
| | | | | Feb. 22, 2021 | | 91 | 90 |
| | | | | Mar. 1, 2021 | | 94 | 96 |
| | | | | Mar. 9, 2021 | | 97 | 97 |
| | | | | Mar. 16, 2021 | | 93 | 93 |
| | | | | Mar. 31, 2021 | | 94 | 93 |
| | | | | Apr. 21, 2021 | | 94 | 95 |
| | | | | May 17, 2021 | | 79 | 79 |
| | | | | Jun. 22, 2021 | | 78 | 74 |
| | | | | Jul. 1, 2021 | | 68 | 64 |
| 44 | (−) | None | V1-P: | Dec. 15, 2020 | 26 | | |
| | | | Apr. 5, 2021, | Feb. 16, 2021 | | 32 | 1 |
| | | | V2-P: | Apr. 1, 2021 | | 23 | −3 |
| | | | Apr. 26, 2021 | Apr. 5, 2021 | | | |
| | | | | Apr. 13, 2021 | | 23 | 1 |
| | | | | Apr. 21, 2021 | | 34 | 12 |
| | | | | Apr. 29, 2021 | | 30 | 12 |
| | | | | May 6, 2021 | | 62 | 56 |
| | | | | Jun. 18, 2021 | | 51 | 39 |
| | | | | Jun. 22, 2021 | | 52 | 40 |
| 48 | 0 | None | V1(P) | Jan. 12, 2021 | 17 | | |
| | | | V2 | May 25, 2021 | | 72 | 68 |
| 49 | 0 | None | V1-P: | Jan. 15, 2021 | 10 | 5 | 1 |
| | 0 | | Jan. 7, 2021, | Jan. 22, 2021 | | 22 | 11 |
| | | | V2-P: | Jan. 27, 2021 | | 24 | 14 |
| | | | Jan. 28, 2021 | Feb. 5, 2021 | | 82 | 82 |
| | | | | Feb. 11, 2021 | | 77 | 76 |
| | | | | Feb. 18, 2021 | | 76 | 74 |
| | | | | Mar. 16, 2021 | | 57 | 53 |
| | | | | Apr. 1, 2021 | | 49 | 44 |
| | | | | Apr. 27, 2021 | | 41 | 39 |
| | | | | Jun. 2, 2021 | | 35 | 31 |
| | | | | Jun. 24, 2021 | | 37 | 30 |
| 59 | (+) | Fever/Cough/ | V-?: | Jan. 20, 2021 | | 55 | 57 |
| | | aches | Mar. 19, 2021 | Mar. 18, 2021 | | 57 | 61 |
| | | | | Jun. 18, 2021 | | 99 | 99 |
| 63 | 0 | coughing/ | V1-M: | Jan. 18, 2021 | N/A | | |
| | | sneezing | Dec. 23, 2020, | | | | |
| | | | V2-M: | | | | |
| | | | Jan. 20, 2021 | | | | |
| 64 | 0 | None | V1-M: | Jan. 17, 2021 | N/A | | |
| | | | Jan. 19, 2021, | Jan. 27, 2021 | | 11 | −3 |
| | | | V2-M: | Feb. 1, 2021 | | 19 | 13 |
| | | | Feb. 16, 2021 | Feb. 9, 2021 | | 24 | 17 |
| | | | | Feb. 15, 2021 | | 32 | 27 |
| | | | | Feb. 23, 2021 | | 93 | 94 |
| | | | | Mar. 2, 2021 | | 91 | 92 |
| | | | | May 26, 2021 | | 42 | 41 |
| 65 | 0 | None | V1-M: | Jan. 17, 2021 | N/A | | |
| | | | Jan. 19, 2021, | Jan. 27, 2021 | | −3 | −1 |
| | | | V2-M: | Feb. 1, 2021 | | 9 | 8 |
| | | | Feb. 16, 2021 | Feb. 9, 2021 | | 13 | 7 |
| | | | | Feb. 15, 2021 | | 11 | 6 |
| | | | | Feb. 23, 2021 | | 67 | 69 |
| | | | | Mar. 2, 2021 | | 75 | 78 |
| | | | | Mar. 9, 2021 | | 62 | 64 |
| | | | | May 26, 2021 | | 24 | 22 |
| 76 | 0 | None | V1-P: | Jan. 24, 2021 | | 29 | 1 |
| | | | Feb. 1, 2021, | Feb. 7, 2021 | | 30 | −7 |
| | | | V2-P: | Feb. 15, 2021 | | 45 | 7 |
| | | | Feb. 22, 2021 | Feb. 21, 2021 | | 40 | 11 |
| | | | | Mar. 1, 2021 | | 76 | 72 |
| | | | | Mar. 8, 2021 | | 91 | 89 |
| | | | | May 14, 2021 | | 36 | 27 |
| 79 | 0 | None | V1-M: | Jan. 24, 2021 | | 14 | 0 |
| | | | Jan. 26, 2021, | Feb. 7, 2021 | | 39 | 36 |
| | | | V2-M: | Feb. 15, 2021 | | 35 | 26 |
| | | | Feb. 23, 2021 | Feb. 21, 2021 | | 27 | 18 |
| | | | | Mar. 2, 2021 | | 85 | 86 |
| | | | | Mar. 9, 2021 | | 78 | 81 |
| | | | | May 15, 2021 | | 71 | 71 |
| 80 | (−) | None | V1-M, | Jan. 24, 2021 | | −1 | −3 |
| | | | Jan. 4, 2021, | Feb. 13, 2021 | | 34 | 25 |
| | | | V2-M | Feb. 27, 2021 | | 29 | 20 |
| | | | Feb. 3, 2021 | | | | |
| 84 | (−) | None | V1-M: | Jan. 24, 2021 | | 19 | 2 |
| | | | Feb. 3, 2021 | Feb. 13, 2021 | | 17 | 2 |
| | | | V2, | Feb. 27, 2021 | | 37 | 23 |
| | | | Mar. 2, 2021 | Mar. 13, 2021 | | 94 | 94 |
| 85 | 0 | None | V1-P, | Jan. 24, 2021 | | 27 | −1 |
| | | | Feb. 25, 2021 | Feb. 25, 2021 | | 18 | −2 |
| | | | | Mar. 7, 2021 | | 7 | −1 |
| | | | V2, | Mar. 13, 2021 | | 40 | 16 |
| | | | Mar. 18, 2021 | | | | |
| 89 | 0 | None | V1-M: | Jan. 24, 2021 | | 35 | 11 |
| | | | Jan. 15, 2021, | Feb. 12, 2021 | | 55 | 39 |
| | | | V2-M: | Feb. 19, 2021 | | 90 | 89 |
| | | | Feb. 12, 2021 | Feb. 26, 2021 | | 86 | 87 |
| | | | | May 13, 2021 | | 65 | 56 |
| 92 | 0 | None | V1-P: | Jan. 24, 2021 | | 92 | 93 |
| | | | Dec. 18, 2020, | Feb. 12, 2021 | | 79 | 81 |
| | | | V2-P: | Feb. 19, 2021 | | 75 | 73 |
| | | | Jan. 7, 2021 | May 14, 2021 | | 45 | 31 |
| 94 | (−) | None | V1-M: | Jan. 24, 2021 | | 0 | −1 |
| | | | Jan. 28, 2021, | Feb. 7, 2021 | | 19 | −3 |
| | | | V2-M: | Feb. 15, 2021 | | 50 | 28 |
| | | | Feb. 25, 2021 | Feb. 21, 2021 | | 43 | 21 |
| | | | | Mar. 4, 2021 | | 94 | 94 |
| | | | | Mar. 11, 2021 | | 75 | 78 |
| | | | | May 14, 2021 | | 61 | 54 |
| 97 | 0 | None | V1-M: | Jan. 21, 2021 | | 98 | 96 |
| | | | Dec. 24, 2020, | | | | |
| | | | V2-M: | | | | |
| | | | Jan. 21, 2021 | | | | |
| 98 | 0) | None | V1-M: | Jan. 21, 2021 | | 96 | 96 |
| | | | Dec. 24, 2020, | | | | |
| | | | V2-M: | | | | |
| | | | Jan. 21, 2021 | | | | |
| 100 | (−) | None | V1-M: | Jan. 24, 2021 | | 1 | 3 |
| | | | Feb. 1, 2021 | Feb. 7, 2021 | | −6 | −7 |
| | | | V2, | Feb. 15, 2021 | | 22 | 18 |
| | | | Mar. 4, 2021 | Feb. 21, 2021 | | 32 | 16 |
| | | | | Feb. 28, 2021 | | 18 | 13 |
| | | | | Mar. 11, 2021 | | 79 | 81 |
| | | | | Mar. 18, 2021 | | 89 | 92 |
| | | | | May 17, 2021 | | 73 | 74 |
| 101 | 0 | None | V1-M: | Jan. 24, 2021 | | 11 | 6 |
| | | | Jan. 28, 2021, | Feb. 7, 2021 | | 17 | 5 |
| | | | V2-M: | Feb. 15, 2021 | | 14 | 7 |
| | | | Feb. 25, 2021 | Feb. 21, 2021 | | 16 | 7 |
| | | | | Mar. 4, 2021 | | 42 | 38 |
| | | | | Mar. 11, 2021 | | 46 | 42 |
| | | | | Mar. 18, 2021 | | 45 | 42 |
| | | | | May 17, 2021 | | 26 | 20 |
| 103 | (−) | None | V1(M): | Jan. 26, 2021 | | 9 | 1 |
| | | | Mar. 25, 2021 | Mar. 25, 2021 | | 16 | −2 |
| | | | V2: | Apr. 22, 2021 | | | |
| | | | Apr. 22, 2021 | | | | |
| 112 | 0 | None | J&J: | Jan. 30, 2021 | | 11 | −1 |
| | | | Apr. 6, 2021 | Apr. 10, 2021 | | 15 | 0 |
| | | | | Apr. 18, 2021 | | 28 | 1 |
| 121 | (−) | None | J&J: | Jan. 30, 2021 | | 19 | −2 |
| | | | Apr. 7, 2021 | Apr. 10, 2021 | | 34 | 1 |
| 123 | (−) | None | V1(P), | Jan. 30, 2021 | | −2 | −1 |
| | | | Mar. 9, 2021 | Mar. 13, 2021 | | 17 | 0 |
| | | | V2: | Apr. 10, 2021 | | 77 | 82 |
| | | | Mar. 31, 2021 | | | | |
| 126 | (−) | None | V1-M: | Jan. 30, 2021 | | 7 | −1 |
| | | | Jan. 31, 2021, | Feb. 6, 2021 | | −3 | −1 |
| | | | V2-M | Feb. 27, 2021 | | 52 | 51 |
| | | | Feb. 28, 2021 | | | | |
| 134 | 0 | None | V1-M: | Jan. 30, 2021 | | 26 | 20 |
| | | | Dec. 28, 2020, | Feb. 5, 2021 | | 92 | 92 |
| | | | V2-M: | | | | |
| | | | Jan. 26, 2021 | | | | |
| 139 | 0 | None | V1-M: | Jan. 26, 2021 | | 24 | −2 |
| | | | Jan. 29, 2021 | Feb. 8, 2021 | | 32 | 3 |
| | | | V2, | Feb. 17, 2021 | | 44 | 14 |
| | | | Feb. 26, 2021 | Feb. 26, 2021 | | 35 | 12 |
| | | | | Mar. 5, 2021 | | 85 | 86 |
| | | | | Mar. 12, 2021 | | 88 | 89 |
| | | | | May 19, 2021 | | 77 | 78 |
| 140 | 0 | None | V1-M: | Jan. 25, 2021 | | 40 | 23 |
| | | | Jan. 10, 2021, | Feb. 3, 2021 | | 51 | 32 |
| | | | V2-M: | Feb. 8, 2021 | | 44 | 27 |
| | | | Feb. 8, 2021 | Feb. 16, 2021 | | 99 | 99 |
| | | | | Feb. 22, 2021 | | 86 | 98 |
| | | | | Feb. 22, 2021 | | 98 | 98 |
| | | | | May 10, 2021 | | 87 | 88 |
| 142 | (−) | None | | Nov. 27, 2020 | | 30 | 0 |
| | | | | May 13, 2021 | | 34 | −1 |
| 146 | | | V1-P: | Feb. 4, 2021 | | −4 | −1 |
| | | | Jan. 27, 2021 | Feb. 11, 2021 | | 20 | 13 |
| | | | V2, | Feb. 17, 2021 | | 34 | 30 |
| | | | Feb. 18, 2021 | Feb. 24, 2021 | | 86 | 85 |
| | | | | Mar. 4, 2021 | | 89 | 88 |
| | | | | Mar. 18, 2021 | | 75 | 75 |
| | | | | May 22, 2021 | | 66 | 61 |
| 147 | 0 | None | V1(M): | Feb. 3, 2021 | | 49 | 42 |
| | | | Jan. 10, 2021 | Feb. 8, 2021 | | 46 | 36 |
| | | | V2: | Feb. 15, 2021 | | 97 | 98 |
| | | | Feb. 8, 2021 | Feb. 23, 2021 | | 94 | 94 |
| | | | | May 11, 2021 | | 68 | 63 |
| 149 | 0 | None | V-M, Date? | Feb. 6, 2021 | | 36 | 28 |
| 150 | (−) | None | V1-M: | Feb. 6, 2021 | | 65 | 60 |
| | | | Jan. 9, 2021, | Feb. 27, 2021 | | 90 | 92 |
| | | | V2-M: | Mar. 7, 2021 | | 93 | 93 |
| | | | Feb. 6, 2021 | Apr. 10, 2021 | | 74 | 77 |
| 151 | (−) | None | V1-P: | Feb. 6, 2021 | | 33 | 26 |
| | | | Dec. 1/2021, | | | | |
| | | | V2-P | | | | |
| | | | Jan. 16, 2021 | | | | |
| 165 | (−) | None | V1-M: | Feb. 6, 2021 | | 13 | −3 |
| | | | Jan. 7, 2021, | Mar. 13, 2021 | | 41 | 36 |
| | | | V2-M: | | | | |
| | | | Feb. 4, 2021 | | | | |
| 166 | (+) | Body Aches/ | V1 (M), | Feb. 6, 2021 | | 42 | 27 |
| | | Nausea/Dizzy | Jan. 8, 2021 | Feb. 27, 2021 | | 71 | 69 |
| | | | V2-M: | Mar. 13, 2021 | | 68 | 65 |
| | | | Feb. 5, 2021 | Apr. 10, 2021 | | 60 | 57 |
| 173 | 0 | None | V1-M: | Feb. 9, 2021 | | 23 | −15 |
| | | | Feb. 9, 2021 | Feb. 25, 2021 | | 8 | 5 |
| | | | V2: | May 7, 2021 | | 60 | 52 |
| | | | Mar. 9, 2021 | | | | |
| 175 | 0 | None | V1-M | Feb. 9, 2021 | | 17 | −8 |
| | | | Feb. 9, 2021 | Feb. 25, 2021 | | 8 | 5 |
| | | | V2: | May 7, 2021 | | 42 | 35 |
| | | | Mar. 9, 2021 | | | | |
| 178 | 0 | None | V1-M: | Feb. 8, 2021 | | 46 | 36 |
| | | | Jan. 10, 2021, | Feb. 15, 2021 | | 97 | 98 |
| | | | V2-M: | Feb. 23, 2021 | | 94 | 94 |
| | | | Feb. 8, 2021 | | | | |
| 179 | 0 | None | V1-M: | Feb. 8, 2021 | | −2 | −4 |
| | | | Feb. 12, 2021 | Feb. 19, 2021 | | 14 | −3 |
| | | | V2: | Feb. 26, 2021 | | 4 | 4 |
| | | | Mar. 5, 2021 | Mar. 5, 2021 | | 16 | 6 |
| | | | | Mar. 12, 2021 | | 36 | 29 |
| | | | | Mar. 21, 2021 | | 8 | 7 |
| | | | | Mar. 30, 2021 | | 51 | 44 |
| | | | | Apr. 6, 2021 | | 56 | 59 |
| | | | | Jun. 16, 2021 | | 33 | 19 |
| 180 | 0 | None | V1-P: | Feb. 11, 2021 | | 7 | 1 |
| | | | Feb. 11, 2021 | Feb. 19, 2021 | | 20 | −1 |
| | | | V2: | Mar. 1, 2021 | | 40 | 38 |
| | | | Mar. 4, 2021 | Mar. 7, 2021 | | 41 | 34 |
| | | | | Mar. 30, 2021 | | 91 | 92 |
| 181 | 0 | Coughing/ | V1 | Feb. 11, 2021 | | 98 | 97 |
| | | Difficulty | | | | | |
| | | Breathing/ | | | | | |
| | | Fatigue | | | | | |
| 182 | (−) | None | V1-P: | Feb. 9, 2021 | | −3 | −3 |
| | | | Feb. 9, 2021 | Feb. 18, 2021 | | 27 | −1 |
| | | | V2: | Feb. 25, 2021 | | 26 | 22 |
| | | | Mar. 3, 2021 | Mar. 4, 2021 | | 49 | 37 |
| | | | | Mar. 28, 2021 | | 93 | 93 |
| | | | | Apr. 11, 2021 | | 94 | 94 |
| 183 | 0 | None | V1-P. | Feb. 11, 2021 | | 88 | 87 |
| | | | Jan. 9, 2021, | | | | |
| | | | V2-P | | | | |
| | | | Jan. 25, 2021 | | | | |
| 186 | 0 | None | V1-P: | Feb. 8, 2021 | | 90 | 88 |
| | | | Jan. 8, 2021, | Feb. 20, 2021 | | 99 | 99 |
| | | | V2-P: | | | | |
| | | | Jan. 29, 2021 | | | | |
| 187 | 0 | None | V1-M: | Feb. 16, 2021 | | 88 | 92 |
| | | | Jan. 28, 2021 | | | | |
| 189 | (+) | Cough | V1(M), | Feb. 19, 2021 | | 100 | 99 |
| | | | Feb. 11, 2021 | Feb. 26, 2021 | | 99 | 99 |
| | | | V2: | Mar. 22, 2021 | | 99 | 99 |
| | | | Mar. 12, 2021 | | | | |
| 190 | (+) | Fever, | V1(M), | Feb. 19, 2021 | | 98 | 98 |
| | | difficulty | Feb. 10, 2021 | Feb. 26, 2021 | | 97 | |
| | | breathing, | V2: | Apr. 4, 2021 | | 95 | 93 |
| | | loss of | Mar. 11, 2021 | | | | |
| | | taste/smell | | | | | |
| 191 | (+) | Fever, | V1(M): | Feb. 19, 2021 | | 42 | 31 |
| | | difficulty | Mar. 19, 2021 | Apr. 4, 2021 | | 100 | 99 |
| | | breathing, | | | | | |
| | | loss of | | | | | |
| | | taste/smell | | | | | |
| 192 | (+) | Fever, cough, | V1-M, | Feb. 19, 2021 | | | |
| | | body ache, | Feb. 16, 2021 | Feb. 26, 2021 | | 99 | 99 |
| | | loss of taste | V2: | Mar. 22, 2021 | | 99 | 99 |
| | | & smell | Mar. 22, 2021 | | | | |
| 194 | (−) | Fatigue | V1-M, | Feb. 27, 2021 | | 16 | 5 |
| | | | Feb. 6, 2021 | | | | |
| 195 | (−) | None | V1-M, | Feb. 27, 2021 | | 13 | 9 |
| | | | Feb. 6, 2021 | | | | |
| 196 | (−) | None | V1-M; | Feb. 27, 2021 | | 96 | 97 |
| | | | Jan. 7, 2021, | Mar. 13, 2021 | | 99 | 99 |
| | | | V2-M: | | | | |
| | | | Feb. 4, 2021 | | | | |
| 197 | (−) | None | V1-P; | Feb. 28, 2021 | | 24 | 2 |
| | | | Feb. 14, 2021, | Mar. 7, 2021 | | 24 | 3 |
| | | | V2-P: | Mar. 15, 2021 | | 60 | 49 |
| | | | Mar. 7, 2021 | | | | |
| 198 | (−) | None | V1-M: | Feb. 25, 2021 | | 43 | 27 |
| | | | Feb. 2, 2021 | | | | |
| 200 | | | V1-M: | Mar. 1, 2021 | | 18 | 19 |
| | | | Feb. 10, 2021 | Mar. 10, 2021 | | 18 | 20 |
| | | | V2: | Mar. 17, 2021 | | 69 | 75 |
| | | | Mar. 10, 2021 | Mar. 22, 2021 | | 80 | 85 |
| | | | | Apr. 11, 2021 | | 56 | 60 |
| 203 | 0 | None | V1-M: | Mar. 7, 2021 | | 82 | 84 |
| | | | Jan. 11, 2021, | | | | |
| | | | V2-M: | | | | |
| | | | Feb. 4, 2021 | | | | |
| 208 | | Fatigue | V1-P: | Mar. 7, 2021 | | 36 | 30 |
| | | | Feb. 7, 2021, | | | | |
| | | | V2-P: | | | | |
| | | | Feb. 28, 2021 | | | | |
| 209 | 0 | Difficult | V1-P: | Mar. 7, 2021 | | 49 | 38 |
| | | breathing | Feb. 20, 2021 | Mar. 13, 2021 | | 55 | 43 |
| | | | V2: | Apr. 10, 2021 | | 51 | 46 |
| | | | Mar. 12, 2021 | Apr. 18, 2021 | | 55 | 42 |
| 210 | (+) | Fever/ | V1-M: | Mar. 7, 2021 | | 100 | 99 |
| | | difficulty | Jan. 7, 2021, | Apr. 10, 2021 | | 92 | 93 |
| | | breathing, | V2-M: | | | | |
| | | body | Feb. 4, 2021 | | | | |
| | | aches/fatigue | | | | | |
| 211 | 0 | None | V1-P: | Mar. 7, 2021 | | 6 | 4 |
| | | | Feb. 19, 2021 | Mar. 13, 2021 | | 30 | 5 |
| | | | V2: | Apr. 18, 2021 | | 59 | 48 |
| | | | Mar. 12, 2021 | | | | |
| 212 | (+) | Fever/ | V1-P: | Mar. 7, 2021 | | 27 | 4 |
| | | coughing/ | Feb. 19, 2021 | Mar. 13, 2021 | | 32 | 3 |
| | | difficulty | V2, | Apr. 18, 2021 | | 63 | 52 |
| | | breathing/ | Mar. 12, 2021 | | | | |
| | | body aches/ | | | | | |
| | | fatigue/loss | | | | | |
| | | of taste and | | | | | |
| | | smell | | | | | |
| 213 | (+) | | V1(M): | Mar. 7, 2021 | | 29 | 19 |
| | | | Mar. 3, 2021 | Mar. 30, 2021 | | 99 | 99 |
| 214 | | | | Mar. 5, 2021 | | 91 | 92 |
| 215 | | | V1-P: | Mar. 10, 2021 | | 5 | −3 |
| | | | Mar. 3, 2021 | Mar. 17, 2021 | | 23 | 0 |
| | | | V2: | Mar. 24, 2021 | | 14 | 1 |
| | | | Mar. 24, 2021 | Mar. 31, 2021 | | 37 | 21 |
| | | | | Apr. 7, 2021 | | 39 | 25 |
| | | | | Apr. 14, 2021 | | 45 | 33 |
| | | | | May 12, 2021 | | 26 | 21 |
| | | | | Jun. 9, 2021 | | | |
| 216 | | | V1-P: | Mar. 10, 2021 | | 19 | 2 |
| | | | Mar. 3, 2021 | Mar. 17, 2021 | | 24 | 2 |
| | | | V2: | Mar. 24, 2021 | | 23 | 5 |
| | | | Mar. 24, 2021 | Mar. 31, 2021 | | 50 | 36 |
| | | | | Apr. 7, 2021 | | 56 | 43 |
| | | | | Apr. 14, 2021 | | 53 | 43 |
| | | | | May 12, 2021 | | 46 | 31 |
| | | | | Jun. 9, 2021 | | | |
| 220 | 0 | None | V1(P), | Mar. 13, 2021 | | 56 | 39 |
| | | | Feb. 23, 2021 | | | | |
| | | | V2, | | | | |
| | | | Mar. 16, 2021 | | | | |
| 221 | 0 | Coughing/ | V1(P), | Mar. 13, 2021 | | 14 | 1 |
| | | difficulty | Mar. 15, 2021 | | | | |
| | | breathing/ | | | | | |
| | | body aches/ | | | | | |
| | | loss of taste | | | | | |
| 223 | | | V1-P: | Mar. 16, 2021 | | 84 | 81 |
| | | | Feb. 6, 2021, | | | | |
| | | | V2-P: | | | | |
| | | | Mar. 2, 2021 | | | | |
| 224 | 0 | None | V1(P), | Mar. 13, 2021 | | 31 | 25 |
| | | | Feb. 8, 2021 | | | | |
| | | | V2, | | | | |
| | | | Mar. 1, 2021 | | | | |
| 225 | 0 | None | V(J&J) | Mar. 14, 2021 | | 8 | −4 |
| | | | Mar. 15, 2021 | Mar. 21, 2021 | | 9 | 0 |
| | | | | Mar. 28, 2021 | | 7 | 0 |
| | | | | Apr. 5, 2021 | | | |
| | | | | Apr. 10, 2021 | | 14 | 3 |
| | | | | Apr. 19, 2021 | | 16 | 5 |
| | | | | Apr. 30, 2021 | | | |
| | | | | May 9, 2021 | | 17 | 11 |
| | | | | May 31, 2021 | | 20 | 13 |
| | | | | Jul. 11, 2021 | | 16 | 11 |
| 226 | 0 | None | V1(P): | Mar. 14, 2021 | | 5 | −3 |
| | | | Mar. 16, 2021 | Mar. 21, 2021 | | 7 | 1 |
| | | | V2P): | Mar. 28, 2021 | | 12 | 4 |
| | | | Apr. 6, 2021 | Apr. 5, 2021 | | | |
| | | | | Apr. 10, 2021 | | 84 | 75 |
| | | | | Apr. 19, 2021 | | 99 | 99 |
| | | | | Apr. 30, 2021 | | | |
| | | | | May 9, 2021 | | 92 | 90 |
| | | | | May 31, 2021 | | 88 | 84 |
| | | | | Jul. 11, 2021 | | 64 | 58 |
| 227 | 0 | None | V1-P: | Mar. 13, 2021 | | 97 | 97 |
| | | | Feb. 3, 2021, | | | | |
| | | | V2-P: | | | | |
| | | | Feb. 24, 2021 | | | | |
| 228 | | | ? | Mar. 13, 2021 | | 43 | 29 |
| 229 | 0 | None | V1-P: | Mar. 12, 2021 | | 57 | 43 |
| | | | Feb. 11, 2021, | | | | |
| | | | V2-P: | | | | |
| | | | Mar. 4, 2021 | | | | |
| 230 | | | V1-M: | Mar. 13, 2021 | | 49 | 46 |
| | | | Mar. 12, 2021? | | | | |
| 232 | | | V1-P: | Mar. 16, 2021 | | 84 | 81 |
| | | | Feb. 6, 2021, | | | | |
| | | | V2-P: | | | | |
| | | | Mar. 2, 2021 | | | | |
| 233 | | | V1-M: | Mar. 17, 2021 | | 70 | 72 |
| | | | Jan. 27, 2021, | | | | |
| | | | V2-M: | | | | |
| | | | Feb. 24, 2021 | | | | |
| 234 | | | V1(M): | Mar. 21, 2021 | | 82 | 84 |
| | | | Jan. 24, 2021 | | | | |
| | | | V2: | | | | |
| | | | Feb. 24, 2021 | | | | |
| 237 | | | V1-M: | Mar. 17, 2021 | | 49 | 45 |
| | | | Jan. 27, 2021, | | | | |
| | | | V2-M: | | | | |
| | | | Feb. 24, 2021 | | | | |
| 243 | | | V1(P): | Mar. 28, 2021 | | 25 | 18 |
| | | | Mar. 7, 2021 | | | | |
| | | | V2: | | | | |
| | | | Mar. 28, 2021 | | | | |
| 244 | | | V1-M: | Apr. 1, 2021 | | 87 | 88 |
| | | | Jan. 9, 2021, | | | | |
| | | | V2-M: | | | | |
| | | | Feb. 11, 2022 | | | | |
| 251 | | | V1(M): | Mar. 30, 2021 | | 78 | 66 |
| | | | Mar. 3, 2021 | | | | |
| 254 | | | V1-M: | Apr. 10, 2021 | | 11 | 2 |
| | | | Mar. 19, 2021 | Apr. 18, 2021 | | 13 | 1 |
| 255 | | | V1-M: | Apr. 8, 2021 | | 100 | 99 |
| | | | Dec. 31, 2020, | | | | |
| | | | V2-M: | | | | |
| | | | Jan. 28, 2021 | | | | |
| 256 | | | J&J | Apr. 21, 2021 | | 8 | 2 |
| | | | Mar. 29, 2021 | May 22, 2021 | | 24 | 7 |
| 258 | | | | Apr. 29, 2021 | | 86 | 84 |
| 259 | | | | Apr. 29, 2021 | | 42 | 36 |
| 260 | | | | May 22, 2021 | | 50 | 32 |
| 261 | | | | May 18, 2021 | | 25 | 2 |
| 262 | | | V1(M)/ | May 29, 2021 | | 82 | 82 |
| | | | V2: >6 wks | | | | |
| 263 | | | V? | May 29, 2021 | | 94 | 96 |
| 264 | | | V1(P): | May 29, 2021 | | 33 | 33 |
| | | | Feb. 3, 2021 | | | | |
| | | | V2: | | | | |
| | | | Feb. 24, 2021 | | | | |
| 265 | | | V1(M)/ | May 29, 2021 | | 63 | 65 |
| | | | V2: >6 wks | | | | |
| 266 | | | V1(P)/ | May 29, 2021 | | 46 | 36 |
| | | | V2: >6 wks | | | | |
| 267 | | | V1(M): | May 29, 2021 | | 39 | 33 |
| | | | Jan. 20, 2021 | | | | |
| | | | V2: | | | | |
| | | | Feb. 22, 2021 | | | | |
| 268 | | | J&J: | May 18, 2021 | | 64 | 66 |
| | | | May 10, 2021 | | | | |
| 276 | | | V-AZ1: | Jun. 14, 2021 | | | |
| | | | Jan. 14, 2021 | | | | |
| | | | V1(M): | | | | |
| | | | Jan. 28, 2021 | | | | |
| | | | V2: | | | | |
| | | | Feb. 26, 2021 | | | | |
| 279 | | | J&J | Jun. 14, 2021 | | 24 | 6 |
| | | | Mar. 12, 2021 | | | | |

TABLE 8
SARS-COV-2 Ab Random Sample Test Result - Convalescent Patients

| | | | | | NAb % | | |
|---|---|---|---|---|---|---|---|
| | | | | | Single | Triple- | |
| Sample | RT- | | | | Bead | Bead | |
| ID | PCR | Symptom | Symptom Date | Sample Date | RBD | RBD | S1 |
| 2 | + | − | | Dec. 29, 2020 | 51 | | |
| 10 | + | + | Dec. 20, 2020- | Dec. 29, 2020 | 24 | | |
| | | | Dec. 22, 2020 | Jan. 11, 2021 | 35 | | |
| | | | | Mar. 1, 2021 | | 37 | 13 |
| 13 | − | + | | Dec. 29, 2020 | 70 | | |
| | | | | Jan. 12, 2021 | 74 | | |
| | | | | Feb. 19, 2021 | | 66 | 52 |
| | | | | Feb. 19, 2021 | | 66 | 52 |
| 22 | − | − | | Nov. 27, 2020 | 21 | 27 | 15 |
| | | | | Dec. 21, 2020 | 23 | 28 | 18 |
| | + | + | Christmas | Dec. 31, 2020 | 100 | 100 | 99 |
| | | | Eve | Feb. 9, 2021 | | 95 | 96 |
| | | | | Mar. 10, 2021 | | 86 | 87 |
| | | | | Apr. 7, 2021 | | 80 | 81 |
| | | | | Apr. 21, 2021 | | 81 | 85 |
| 29 | (+) | Fever/ | November | Jan. 11, 2021 | 48 | | |
| | | coughing/ | | Mar. 17, 2021 | | 41 | 26 |
| | | difficulty | | May 10, 2021 | | 38 | 22 |
| | | breathing/ | | | | | |
| | | body aches/ | | | | | |
| | | sneezing/ | | | | | |
| | | fatigue/loss | | | | | |
| | | of taste and | | | | | |
| | | smell | | | | | |
| 31 | 0 | Cough/sneeze | 2 days | Jan. 11, 2021 | 32 | 28 | 10 |
| | | | | Jan. 22, 2021 | | 32 | 8 |
| 33 | (−) | Yes | 19-Dec | Jan. 12, 2021 | 75 | | |
| 35 | 0 | None | N/A | Jan. 12, 2021 | 58 | | |
| | | | | Mar. 13, 2021 | 59 | 63 | 53 |
| 38 | (+) | Fever | 1 day | Jan. 11, 2021 | 19 | | |
| | | | (Jan. 5, 2021) | | | | |
| | (−) | | | Jan. 12, 2021 | 26 | | |
| | | | | Jan. 14, 2021 | 28 | | |
| | | | | Jan. 19, 2021 | 39 | | |
| | | | | Jan. 25, 2021 | 27 | | |
| | | | | Feb. 1, 2021 | | 25 | 8 |
| | | | | May 25, 2021 | | 26 | 4 |
| | | | | Jun. 4, 2021 | | | |
| | | | | Jun. 11, 2021 | | | |
| | | | | Jun. 18, 2021 | | 96 | 98 |
| | | | | Jul. 2, 2021 | | 97 | 98 |
| 52 | (+) | Body aches, | Aug. 26, 2020 | Jan. 17, 2021 | 5 | | |
| | | sneezing, loss | | | | | |
| | | of taste | | | | | |
| | | and/or smell | | | | | |
| 55 | (+) | Fever, | | Jan. 17, 2021 | 23 | | |
| | | difficulty | | | | | |
| | | breathing, | | | | | |
| | | Unusual | | | | | |
| | | recent | | | | | |
| | | fatigue, Loss | | | | | |
| | | of taste | | | | | |
| | | and/or smell | | | | | |
| 77 | 0 | Cough | Thanksgiving | Jan. 24, 2021 | | 33 | 21 |
| 81 | (−) | Cough | Jan. 24, 2021 | Jan. 24, 2021 | | 41 | 30 |
| 86 | (+) | Fever/Cough/ | N/A | Jan. 22, 2021 | | 50 | 37 |
| | | Fatigue/Loss | | | | | |
| | | of Taste | | | | | |
| 87 | (+) | Fever/Cough/ | N/A | Jan. 22, 2021 | | 41 | 19 |
| | | Difficulty | | | | | |
| | | Breath/ | | | | | |
| | | Sneezing/ | | | | | |
| | | Fatigue/Loss | | | | | |
| | | of Taste | | | | | |
| 93 | (+) | Fever/ | N/A | Jan. 24, 2021 | | 66 | 61 |
| | | Difficulty | | Mar. 18, 2021 | | 56 | 48 |
| | | Breath/Aches/ | | May 14, 2021 | | 40 | 32 |
| | | Fatigue | | | | | |
| 104 | | | | Jan. 26, 2021 | | 89 | 80 |
| 105 | | | | Jan. 26, 2021 | | 46 | 24 |
| 113 | 0 | None | N/A | Jan. 30, 2021 | | 34 | 31 |
| 115 | (+) | fever/ | | Jan. 30, 2021 | | 19 | 0 |
| | | coughing/ | | Feb. 6, 2021 | | 50 | 30 |
| | | breathing/ | | | | | |
| | | aches/fatigue | | | | | |
| 117 | (+) | Fever/Cough/ | | Jan. 30, 2021 | | 33 | 6 |
| | | Aches/ | | | | | |
| | | Fatigue | | | | | |
| 119 | (+) | Fever/Aches/ | | Jan. 30, 2021 | | 38 | 22 |
| | | Fatigue/ | | | | | |
| | | Sneezing/ | | | | | |
| | | Loss of taste | | | | | |
| | | and smell | | | | | |
| 122 | (−) | Difficulty | N/A | Jan. 30, 2021 | | 32 | 22 |
| | | breathing | | | | | |
| 125 | 0 | None | N/A | Jan. 30, 2021 | | 25 | −3 |
| | | | | Mar. 13, 2021 | | 20 | −6 |
| 129 | (−) | Aches/Chest | Jan. 15, 2021 | Jan. 30, 2021 | | 55 | 42 |
| | | congestion | | | | | |
| 132 | (+) | Head Aches/ | ? | Jan. 30, 2021 | | 52 | 38 |
| | | Coughing/ | | | | | |
| | | Chest | | | | | |
| | | pain/Aches/ | | | | | |
| | | Fatigue | | | | | |
| 135 | 0 | Fever/ | ? | Jan. 25, 2021 | | 96 | 97 |
| | | Coughing/ | | | | | |
| | | Difficulty | | | | | |
| | | Breathing/ | | | | | |
| | | Aches/ | | | | | |
| | | Fatigue/Loss | | | | | |
| | | of taste and | | | | | |
| | | smell | | | | | |
| 136 | (+) | Fever/ | ? | Jan. 25, 2021 | | 23 | 7 |
| | | Coughing/ | | Mar. 17, 2021 | | 6 | 6 |
| | | Difficulty | | | | | |
| | | Breathing/ | | | | | |
| | | Aches/ | | | | | |
| | | Sneezing/ | | | | | |
| | | Fatigue/Loss | | | | | |
| | | of taste and | | | | | |
| | | smell | | | | | |
| 137 | (+) | Fever/ | ? | Jan. 25, 2021 | | 22 | 12 |
| | | Coughing/ | | | | | |
| | | Loss of taste | | | | | |
| | | and smell | | | | | |
| 142 | | | | Nov. 27, 2020 | | | |
| | | | | Nov. 27, 2020 | | | |
| | | | | Jan. 27, 2021 | | 30 | 0 |
| 147 | | | | Feb. 3, 2021 | | 49 | 42 |
| 149 | 0 | None | N/A | Feb. 6, 2021 | | 36 | 28 |
| 152 | (−) | Fever/ | end of January | Feb. 6, 2021 | | 90 | 87 |
| | | Coughing/ | 2021 | | | | |
| | | Difficulty | | | | | |
| | | Breathing/ | | | | | |
| | | Body Aches/ | | | | | |
| | | Fatigue | | | | | |
| 153 | (+) | Fever/ | | Feb. 6, 2021 | | 65 | 59 |
| | | Coughing/ | | | | | |
| | | Difficulty | | | | | |
| | | Breathing/ | | | | | |
| | | Body Aches/ | | | | | |
| | | Fatigue | | | | | |
| 155 | (−) | None | N/A | Feb. 6, 2021 | | 16 | 10 |
| 157 | (−) | None | N/A | Feb. 6, 2021 | | 50 | 47 |
| 158 | (+) | Fever/ | | Feb. 6, 2021 | | 40 | 38 |
| | | Coughing/ | | | | | |
| | | Difficulty | | | | | |
| | | Breathing/ | | | | | |
| | | Body Aches/ | | | | | |
| | | Fatigue | | | | | |
| 160 | (+) | Fever/ | N/A | Feb. 6, 2021 | | 6 | 0 |
| | | Coughing/ | | | | | |
| | | Difficulty | | | | | |
| | | Breathing/ | | | | | |
| | | Body Aches/ | | | | | |
| | | Sneezing/ | | | | | |
| | | Fatigue | | | | | |
| 161 | (+) | None | N/A | Feb. 6, 2021 | | 16 | 1 |
| | | | | Apr. 10, 2021 | | 19 | −1 |
| 163 | (+) | Coughing/ | N/A | Feb. 6, 2021 | | 23 | 10 |
| | | Difficulty | | | | | |
| | | Breathing/ | | | | | |
| | | Body Aches/ | | | | | |
| | | Fatigue/Loss | | | | | |
| | | Taste and | | | | | |
| | | Smell | | | | | |
| 164 | (+) | Fever/Body | N/A | Feb. 6, 2021 | | 61 | 59 |
| | | Aches/ | | | | | |
| | | Nausea/Dizzy | | | | | |
| 167 | (+) | Head Ache | N/A | Feb. 6, 2021 | | 80 | 67 |
| 168 | (−) | Fever/Body | N/A | Feb. 6, 2021 | | 37 | 30 |
| | | Aches | | | | | |
| 169 | (+) | Headache/ | N/A | No Date | | 25 | 9 |
| | | Lost Taste/ | | | | | |
| | | Running | | | | | |
| | | Nose/ | | | | | |
| | | Congestion | | | | | |
| 171 | (+) | Stuffy Nose | November 2020 | Feb. 5, 2021 | | 54 | 37 |
| 172 | (+) | Fever/ | November 2020 | Feb. 6, 2021 | | 35 | 33 |
| | | Coughing/ | | | | | |
| | | Difficulty | | | | | |
| | | Breathing/ | | | | | |
| | | Lostsof Taste | | | | | |
| | | and Smell | | | | | |
| 174 | (−) | None | Coughing/ | Feb. 9, 2021 | | 10 | 10 |
| | | | Difficult | | | | |
| | | | breathing | | | | |
| 213 | | | | Mar. 7, 2021 | | 29 | 19 |
| 239 | (+) | Fever/ | November | Mar. 20, 2021 | | 19 | 13 |
| | | coughing/ | 2020 | | | | |
| | | difficulty | | | | | |
| | | breathing/ | | | | | |
| | | body aches/ | | | | | |
| | | sneezing/ | | | | | |
| | | fatigue/loss | | | | | |
| | | of taste and | | | | | |
| | | smell | | | | | |
| 240 | (+) | Fever/ | November 2020 | Mar. 20, 2021 | | 4 | 5 |
| | | coughing/ | | | | | |
| | | difficulty | | | | | |
| | | breathing/ | | | | | |
| | | body aches/ | | | | | |
| | | sneezing/ | | | | | |
| | | fatigue/loss | | | | | |
| | | of taste and | | | | | |
| | | smell | | | | | |
| 241 | | | March 2020 | Mar. 18, 2021 | | 52 | 36 |
| | | | | Jun. 17, 2021 | | 46 | 30 |
| 242 | | | | Mar. 16, 2021 | | 67 | 65 |
| 248 | | Cough | April 2020 | Apr. 4, 2021 | | 3 | 1 |
| | | | | May 18, 2021 | | 10 | 1 |
| 249 | | Cough | April 2020 | Apr. 4, 2021 | | 30 | 0 |
| | | | | May 18, 2021 | | 32 | 2 |
| 250 | | Cough | April 2020 | Apr. 4, 2021 | | 26 | 1 |
| | | | | May 18, 2021 | | 23 | 0 |
| 252 | (+) | | | Apr. 10, 2021 | | 47 | 37 |

### Example 14

### Commercial Three-Microparticle Multiplex Assay for Detection of SARS-CoV-2 Neutralizing Antibodies

A commercial three-microparticle multiplex assay of the Platform 3 type is provided as follows. Reagents: a) SARS-CoV-2 Antigen Conjugated Beads Mix (three types of identifiably labelled antibodies, including S1, RBD, and NP antigens); b) NAb Stain Buffer; c) NAb Stain; d) 1% BSA/PBS; e) PBS. Sample: Plasma or Serum (1:20 diluted in 1% BSA/PBS)

### Procedure:

1. Add 5 μL of Antigen Conjugated Beads Mix containing S1, RBD and NP protein coupled beads to each well.
2. Add 20 μL 1% BSA or 20 μL diluted plasma into corresponding wells and shake for 30 second at speed 2 on a titer plate shaker.
3. Add 20 μL NAb Stain Buffer to each well and shake for 30 second at speed 2 on a titer plate shaker.
4. Add 5 μL diluted NAb Stain to each well.
5. Incubate the plate at RT for 60 min with shaking at speed 2 on a titer plate shaker.
6. Add 150 μL 1% BSA/PBS to each well and centrifuge the plate at 2500 g for 3 min.
7. Flick and shake the plate at speed 2 on a titer plate shaker for 30 second.
8. Add 75 μL 1% BSA/PBS to each well and shake the plate at speed 2 on titer plate shaker for 30 second.
9. Add additional 75 μL 1% BSA/PBS to each well and centrifuge the plate at 2500 g for 3 min.
10. Flick and shake the plate at speed 2 on a titer plate shaker for 30 second.
11. Resuspend the beads in 60 μL PBS and acquire the samples on a Lyric Flow Cytometer.
12. Analyze data on NAbs detection template.
13. Calculate the NAb % by the following formula:

NAb (%)=[1% BSA/PBS (MFI)−Sample (MFI)]/1% BSA/PBS (MFI) X %

### Example 15

### Multiplex Assay for Detection of SARS-CoV-2 Neutralizing Antibodies for SARS-CoV-2 Variants Using Multi-Variant Microparticles

A multiplex assay system of the Platform 4 type was used to detect both antibodies (Ab) and neutralizing antibodies (NAb) for SARS-CoV-2 variants. Receptor-binding domain (RBD) proteins of SARS-CoV-2 wild type (WT) and six variants including Afar (α), Beta (β), Gamma (γ), Delta (δ), Epsilon (ε), and Omicron (o) were conjugated to different UV ID beads (beads with distinct UV signatures, each signature corresponding to a bead type conjugated to protein from a different variant) to form a seven multiplex detection assay. A total of 91 samples from 4 different groups with characteristics set forth in Table 9 were tested for SARS-CoV-2 Ab and NAb.

TABLE 9

| Group | Description | N |
|---|---|---|
| Negative | Samples collected three years ago (prior to SARS- | 16 |
| | CoV-2) | |
| Convalescent | RT-PCT confirmed SARS-CoV-2 wild type infected | 30 |
| | patients | |
| Vaccinated | Individuals who received two doses of Pfizer or | 31 |
| | Moderna vaccine | |
| Booster | Individuals who received three doses of Pfizer or | 24 |
| | Moderna vaccine | |

For Ab detection, 50 μl of plasma was incubated with 5 μl of assay medium containing UV-ID beads, with each distinct bead type bound to the S protein from a different SARS-CoV-2 variant, at room temperature for 30 minutes; after washes, the beads were resuspended in 100 μl of stain mix containing fluorescent anti-human IgG, IgM, and IgA. After an additional 30 minutes incubation at RT, the beads were washed and acquired in a BD FACSLyric™ Flow Cytometer.

For NAb detection, 50 μL 1% BSA 1:50 diluted plasma was incubated with 5 μL of assay medium containing UV-ID beads, with each distinct bead type bound to the RBD from a different SARS-CoV-2 variant, at room temperature for 30 minutes. The beads were washed three times with 150 μL 1% BSA/PBS and resuspended in 100 μL PE-labeled ACE-2 (PE-ACE-2). After an additional 30-minute incubation at room temperature, the microspheres were washed and acquired in a BD FACSLyric™ Flow Cytometer. The PE fluorescence intensities on all seven UV ID beads were measured. The inhibition % was calculated as follows: Inhibition %=(1−MFI of sample/MFI of PBS)×100%.

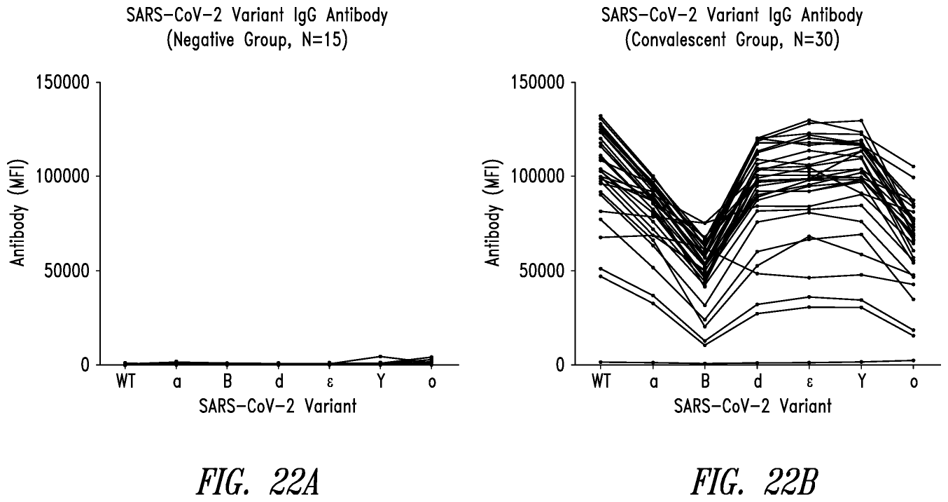

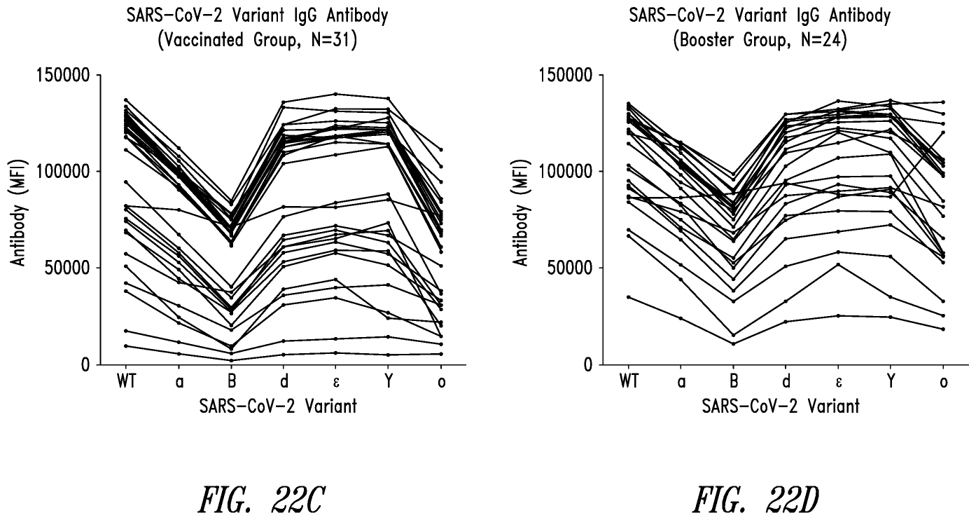

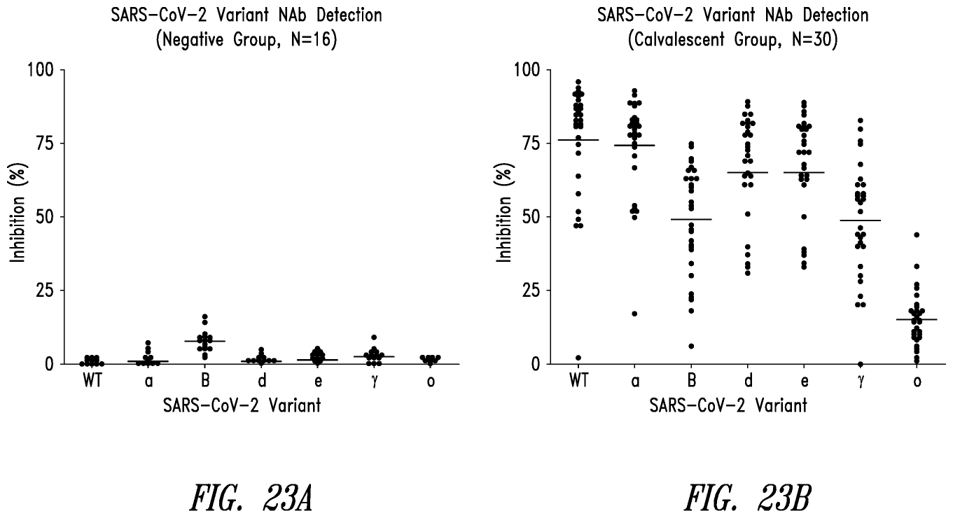

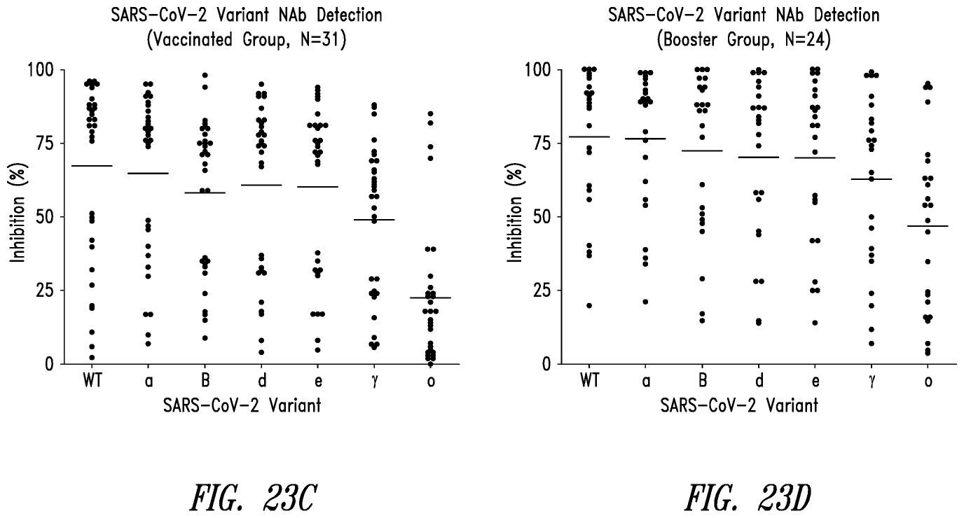

Both SARS CoV-2 Ab and NAb were detected in all convalescent and vaccinated samples. The Abs generated from wild type (WT) infected patients cross-reacted to varying degrees with the other six variant S proteins (FIG. 22A, FIG. 22B, FIG. 22C, FIG. 22D). NAbs also showed differential cross-reactivity between groups, but not proportional to the Ab levels (FIG. 23A, FIG. 23B, FIG. 23C, FIG. 23D). Compared with the 2-dose vaccinated group, both Ab and NAb in the booster group were elevated, consistent with other reports that a third vaccine does boosts immunity to all SARS-CoV-2 variants, including Omicron. NAb results also suggested that individuals who were naturally infected, or received only two vaccine doses, might not be protected against the Omicron variant, consistent with reported clinical findings.

### Example 16

### Confirmatory Assays for Detection of SARS-CoV-2 Neutralizing Antibodies

### Materials Used:

Biotinylated Human ACE2/ACEH Protein, Fc,Avitag™ ACRO Biosystems 310 μg/mL, 25 μg
UV2-RBD beads Conjugated by HL
Biotinylated RBD by HL
P1-ACE2(Acro) Conjugated by HL
SAPE Jackson ImmunoResearch rehydrated in 1 mL Molecular Biology Grade Water
Negative sample: 428
Positive samples: 610195(CCP), 610223(CCP), 314 and 965

### Procedure 1:

1. Add 3000 UV2-RBD beads (5 μL) to each well.
2. Add 20 μL 1% BSA or 20 μl diluted plasma (diluted in 1% BSA/PBS; equal to 0.5 μL/T and 1 μL/T) and 20 μL (0.02 μg) Bio-ACE2 (diluted in 1% BSA/PBS) into corresponding wells and shake for 30 second.
3. Add 5 μL/T diluted SAPE (0.1 μL/T; diluted in 1% BSA/PBS).
4. Incubate 60 min at RT with shaking at speed 2 on titer plate shaker.
5. Wash twice with 150 μL 1% BSA/PBS and cf at 2500 g for 3 min every wash.
6. After each wash, flick and vortex at speed 2 on titer plate shaker for 30 second (Then add 75 μL 1% BSA/PBS and shake 30 sec-add additional 75 μL).
7. Resuspend the beads in 60 μL DPBS and run on flow.

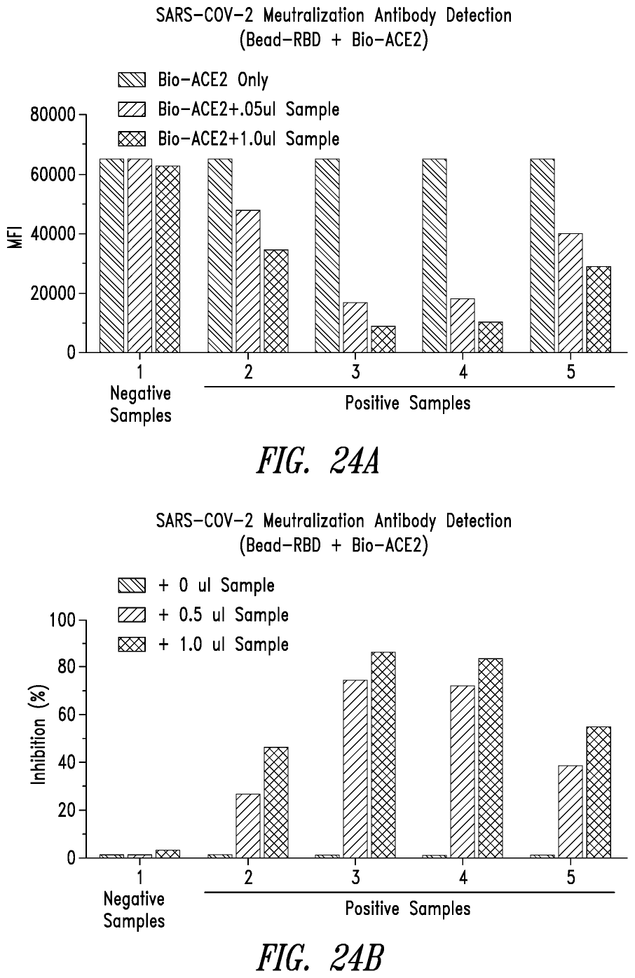

Results for Procedure 1 are presented in FIG. 24A and FIG. 24B and Table 10.

TABLE 10
Neutralizing Ab detection-12/28/20-1 step

| | Bead-P2-RBD-Biotinylated-ACE2(Acro) | | | |
|---|---|---|---|---|
| | 0.5 uL | | 1 uL | |
| Plasma# | MFI | inhibition(%) | MFI | inhibition(%) |
| Bio-ACE2 only | (65405 + 64748)/2 = 65077 | | | |
| 428 | 64967 | 0.17 | 63152 | 2.96 |
| 223 | 47723 | 26.67 | 34847 | 46.45 |
| 195 | 16731 | 74.29 | 9116 | 85.99 |
| 314 | 18204 | 72.03 | 10626 | 83.67 |
| 965 | 39948 | 38.61 | 29123 | 55.25 |

| | |
|---|---|
| Beads only: 50/58 | SAPE(0.1 μL/T) |

### Procedure 2:

1. Add 3000 P1-ACE2 beads (5 μL) to each well.
2. Add 20 μL 1% BSA or 20 μL diluted plasma (diluted in 1% BSA/PBS; equal to 0.5 μL/T and 1 μL/T) and 20 μL (0.02 μg) Bio-RBD (diluted in 1% BSA/PBS) into corresponding wells and shake for 30 second.
3. Add 5 μL/T diluted SAPE (0.1 μL/T; diluted in 1% BSA/PBS).
4. Incubate 60 min at RT with shaking at speed 2 on titer plate shaker.
5. Wash twice with 150 μL 1% BSA/PBS and cf at 2500 g for 3 min every wash.
6. After each wash, flick and vortex at speed 2 on titer plate shaker for 30 second (Then add 75 μL 1% BSA/PBS and shake 30 sec-add additional 75 μL).
7. Resuspend the beads in 60 μL DPBS and run on flow.

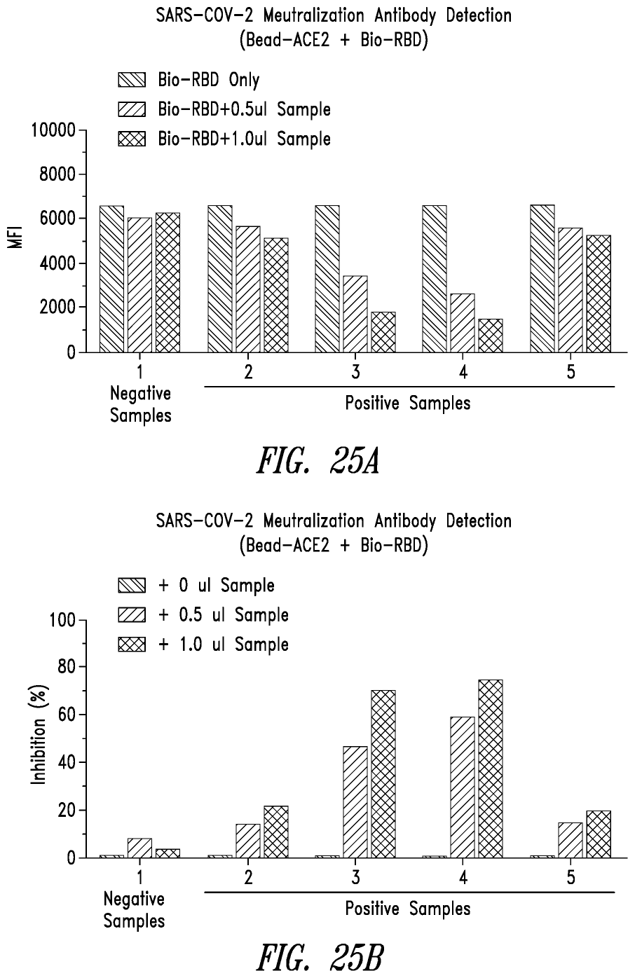

Results for Procedure 2 are presented in FIG. 25A and FIG. 25B and Table 11.

TABLE 11
Neutralizing Ab detection-12/28/20-1 step

| | Bead-P1-ACE-2-Biotinylated-RBD(Exonbio) | | | |
|---|---|---|---|---|
| | 0.5 uL | | 1 uL | |
| Plasma# | MFI | inhibition(%) | MFI | inhibition(%) |
| Bio-RBD only | (6649 + 6526)/2 = 6588 | | | |
| 428 | 6027 | 8.52 | 6334 | 3.86 |
| 223 | 5620 | 14.69 | 5153 | 21.78 |
| 195 | 3419 | 48.1 | 1866 | 71.68 |
| 314 | 2627 | 60.12 | 1575 | 76.09 |
| 965 | 5620 | 14.69 | 5273 | 19.96 |

| | |
|---|---|
| Beads only: 113/115 | SAPE(0.1 μL/T) |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 63/170,130, filed on Apr. 2, 2021 and U.S. Provisional Patent Application No. 63/283,161, filed on Nov. 24, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-82. (canceled)

83. A method of detecting SARS-CoV-2 neutralizing antibodies, the method comprising:
a) combining:
i) at least two types of identifiably labelled microparticles conjugated to at least two different SARS-CoV-2 proteins or a fragment thereof, at least one of which comprises a SARS-CoV-2 S protein or fragment thereof, with
ii) a detectably labelled SARS-CoV-2 S protein receptor or a fragment thereof, and
ii) a test sample;
b) detecting the identifiable labels and the detectable label both associated with the microparticles to generate detection data; and
c) combining or measuring the detection data to generate a test sample property relating to a presence or absence of or amount of SARS-CoV-2 neutralizing antibodies in the test sample.

84. The method of claim 83, wherein the identifiably labelled microparticles include a first type of microparticle conjugated to a first fragment of SARS-CoV-2 S protein, a second type of microparticle conjugated to a second fragment of SARS-CoV-2 S protein, and a third type of microparticle conjugated to SARS-CoV-2 nucleoprotein (NP) protein or a fragment thereof, and, optionally a fourth type of microparticle conjugated to a full-length SARS-CoV-2 S protein.

85. The method of claim 83, wherein the microparticles are identifiable by size, magnetic properties, fluorescence, ultraviolet-excited fluorescence wavelength, violet-excited fluorescence wavelength, fluorescence intensity, metal isotopes, or any combination thereof.

86. The method of claim 83, wherein the SARS-CoV-2 S protein or fragment thereof is subunit 1 (S1) or a fragment thereof or receptor binding domain (RBD) or a fragment thereof.

87. The method of claim 83, wherein the detectably labelled SARS-CoV-2 S protein receptor or fragment thereof is a) detectably labelled with a fluorescent molecule, optionally phycoerythrin, or b) biotinylated and is detected with a streptavidin-labelled fluorescent molecule, optionally streptavidin-phycoerythrin.

88. The method of claim 83, wherein the SARS-CoV-2 S protein receptor or fragment thereof is human angiotensin-converting enzyme 2 (ACE-2) or a fragment thereof.

89. The method of claim 83, wherein the detecting step is carried out using flow cytometry or mass cytometry; or wherein the test sample is whole blood, serum, plasma, nasal secretions, sputum, bronchial lavage, urine, stool, or saliva.

90. The method of claim 83, comprising using the test sample property to provide a diagnosis for a subject who provided the test sample, optionally a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies.

91. The method of claim 83, further comprising an additional SARS-CoV-2 protein or a fragment thereof associated with a SARS-CoV-2 variant.

92. A method of detecting SARS-CoV-2 neutralizing antibodies, the method comprising:
a) one of
i) combining:
A) at least one identifiably labelled microparticle conjugated to a SARS-CoV-2 S protein or a fragment thereof with
B) a detectably labelled SARS-CoV-2 S protein receptor or a fragment thereof, and
C) a test sample, or
ii) combining:
A) identifiably labelled microparticles conjugated to a SARS-CoV-2 S protein receptor or a fragment thereof with
B) a detectably labelled SARS-CoV-2 S protein or a fragment thereof, and
C) a test sample;
b) detecting an identifiable label and a detectable label, both associated with microparticles, to generate detection data; and
c) combining or measuring the detection data to generate a test sample property relating to the presence or absence of or amount of SARS-CoV-2 neutralizing antibodies in the test sample.

93. The method of claim 92, wherein:
a) the SARS-CoV-2 S protein or fragment thereof is subunit 1 (S1) or a fragment thereof or receptor binding domain (RBD) or a fragment thereof,
b) wherein the SARS-CoV-2 S protein receptor or fragment thereof is human angiotensin-converting enzyme 2 (ACE-2) or a fragment thereof; and/or
c) step a) i) A) further comprises combining a second identifiably labelled microparticle conjugated to another SARS-CoV-2 S protein or a fragment thereof or SARS-CoV-2 nucleoprotein (NP) or a fragment thereof with the detectably labelled SARS-CoV-2 S protein or fragment thereof and the test sample.

94. The method of claim 92, wherein the detectably labelled SARS-CoV-2 S protein receptor or fragment thereof is a) detectably labelled with a fluorescent molecule, optionally phycoerythrin, or b) biotinylated and is detected with a streptavidin-labelled fluorescent molecule, optionally streptavidin-phycoerythrin.

95. The method of claim 92, wherein the detecting step is carried out using flow cytometry or mass cytometry, or wherein the test sample is whole blood, serum, plasma, nasal secretions, sputum, bronchial lavage, urine, stool, or saliva.

96. The method of claim 92, comprising using the test sample property to provide a diagnosis for a subject who provided the test sample, optionally a diagnosis of no SARS-CoV-2 neutralizing antibodies, low levels of SARS-CoV-2 neutralizing antibodies, medium levels of SARS-CoV-2 neutralizing antibodies, or high levels of SARS-CoV-2 neutralizing antibodies.

97. A composition comprising:
a) a mixture of at least two types of identifiable microparticles:
i) a first type conjugated to a first SARS-CoV-2 S protein or fragment thereof, and
ii) a second type conjugated to a second fragment of SARS-CoV-2 S protein, which is different from the first fragment.

98. The composition of claim 97, wherein the first SARS-CoV-2 S protein or a fragment thereof or the second fragment of SARS-CoV-2 S protein is subunit 1 (S1) or a fragment thereof or receptor binding domain (RBD) or a fragment thereof.

99. The composition of claim 97, further comprising a detectably labelled SARS-CoV-2 S protein receptor of fragment thereof, optionally human angiotensin-converting enzyme 2 (ACE-2) or a fragment thereof.

100. The composition of claim 99, wherein the detectably labelled SARS-CoV-2 S protein receptor or fragment thereof is a) detectably labelled with a fluorescent molecule, optionally phycoerythrin, or b) biotinylated and is detected with a streptavidin-labelled fluorescent molecule, optionally streptavidin-phycoerythrin.

101. The composition of claim 97, further comprising at least one of SARS-CoV-2 neutralizing antibody, a neutralizing antibody stain buffer, a neutralizing antibody stain, 1% BSA/PBS, PBS, or any combinations thereof.

102. The composition of claim 97, further comprising at least one of:
a) a third type of identifiable microparticle conjugated to a third SARS-CoV-2 nucleoprotein (NP) or a fragment thereof;
b) and, further optionally, a fourth type of identifiable microparticle conjugated to a full-length SARS-CoV-2 S protein; and/or
c) an additional SARS-CoV-2 protein or a fragment thereof associated with a SARS-CoV-2 variant.
